(19) United States
(12) Patent Application Publication
(10) Pub. No.: US 2025/0066852 A1
(43) Pub. Date: Feb. 27, 2025

(54) Kit and Method for Analyzing T Cell Receptors from Single T Cells

(71) Applicant: ST. JUDE CHILDREN'S RESEARCH HOSPITAL, INC., Memphis, TN (US)

(72) Inventors: Mikhail POGORELYY, Memphis, TN (US); Anastasia MINERVINA, Memphis, TN (US); Paul THOMAS, Memphis, TN (US)

(21) Appl. No.: 18/721,777

(22) PCT Filed: Dec. 22, 2022
(86) PCT No.: PCT/US2022/053752

§ 371 Date: Jun. 19, 2024

Related U.S. Application Data
(60) Provisional application No. 63/292,522, filed on Dec. 22, 2021.

(51) Int. Cl. C12Q 1/6876

(52) U.S. Cl. CPC C12Q 1/6876; C12Q 2600/16

(57) ABSTRACT

A kit and method for analyzing nucleic acid molecules encoding T cell receptor (TCR) chains from individual T cells are disclosed. In particular, a method for analyzing individual T cells using high-throughput multiplex amplification and deep sequencing of nucleic acids encoding TCRs is provided.

### INTRODUCTION

This application claims benefit of priority to U.S. Provisional Patent Application Ser. No. 63/292,522, filed Dec. 22, 2021, the content of which is incorporated herein by reference in its entirety.

This invention was made with government support under Grant Number AI136514 awarded by the National Institutes of Health. The government has certain rights in the invention.

### BACKGROUND

T cells are defined by a surface T cell receptor (TCR) that mediates recognition of pathogen-associated epitopes, generally via interactions with peptide-major histocompatibility complexes (pMHC). T cell receptors are generated by germline recombinase activating gene (RAG)-mediated rearrangements of the genomic TCR locus, a process termed V(D)J recombination. This process has the potential to generate a significant number of diverse TCRs, with estimates ranging from 10¹⁵ to as high as 10⁶¹ possible receptors that could be generated by recombination, although only a relatively small portion of these is thought to appear in any individual (˜10⁶−10⁸). In mammals, two types of TCRs are possible, αβ and γδ, and different species produce different ratios of cells bearing these receptors. In humans and mice, αβ T cells dominate, representing up to 90% of the T cell compartment.

The pool of T cells that recognizes a specific epitope expresses diverse TCRs. The size of these naïve precursor repertoires has been estimated for various epitopes by limiting dilution techniques and, more recently, by a tetramer-based magnetic enrichment approach, the latter of which finds pool sizes ranging between 50 and 500 naïve cells per epitope, on average. Due to the rounds of expansion that T cells undergo in the thymus during development, it has been assumed that there are multiple naïve cells with identical TCRs. However, sequencing the naïve repertoire of epitope-specific responses in mice has instead shown that most naïve cells contain a unique receptor, with a very low rate of duplicates among cells.

Sequencing the nucleic acids encoding the T cell receptor requires identifying the specific V-region used by the α or β chain and obtaining the complete sequence of the hypervariable CDR3 region, the site of RAG-mediated V(D)J junctional diversity. Due to the availability of TCR Vβ staining reagents in the human and mouse, analyses of the repertoire initially focused solely on the TCRβ chain. Subsequently, two broad approaches to sequencing the TCR repertoire have emerged: single-cell based methods that permit direct pairing of the α and β chains (Dash et al. (2011) J. Clin. Invest. 121:288-295; Wang et al. (2012) Sci. Transl. Med. 4:128ra42; Kim et al. (2012) PLOS One 7:e37338; and Han et al. (2014) Nat. Biotechnol. 32(7):684-692), and deep sequencing-based methods that amplify single chains from pools of cells (Robins et al. (2009) Blood 114:4099-4107; Weinstein et al. (2009) Science 324:807-810; Freeman et al. (2009) Genome Res. 19:1817-1824) where pairing can be achieved through specific sort conditions and algorithmic imputation (Howie et al. (2015) Sci. Transl. Med. 7:301ra131). Single cell multiplex techniques for TCRαβ or TCRγδ profiling have been described (Dash et al. (2017) Nature 547(7661):89-92; Dash et al. (2015) Meth. Mol. Biol. 1343:181-197; Guo et al. (2016) Mol. Ther. Methods Clin. Dev. 3:15054; Guo et al. (2018) Immunity 49(3):531-44; US 2019/0040381 A1). However, a large-scale multiplexing approach adapted to single cell deep sequencing is needed to increase the throughput of single cell TCR profiling.

### SUMMARY OF THE INVENTION

This invention provides a kit for analyzing a T cell receptor (e.g., TCRαβ or TCRγδ) of a single T cell, which includes (a) a first set of primers including a collection of first forward primers (e.g., SEQ ID NOS:1-40 and SEQ ID NOs:42-70; SEQ ID NOS:72-80 and SEQ ID NOs:82-89; SEQ ID NOs:181-203 and SEQ ID NOs:205-223; or SEQ ID NOs:225-229 and SEQ ID NOs:231-243) and a first reverse primer for each chain of the T cell receptor (e.g., SEQ ID NO:41 and SEQ ID NO:71; SEQ ID NO:81 and SEQ ID NO:90; SEQ ID NO:204 and SEQ ID NO:224; or SEQ ID NO:230 and SEQ ID NO:244), said first set of primers amplifying a nucleic acid molecule encoding a portion of the T cell receptor comprising the hypervariable CDR3 region, wherein each first reverse primer hybridizes to a sequence encoding the constant segment of the T cell receptor chain; and (b) a second set of primers comprising a collection of second forward primers (e.g., SEQ ID NOs:91-130 and SEQ ID NOs:132-160; SEQ ID NOs:162-170 and SEQ ID NOs:172-179; SEQ ID NOs:245-267 and SEQ ID NOs:269-287; or SEQ ID NOs:289-293 and SEQ ID NOs:295-307) and a collection of second reverse primers for each chain of the T cell receptor, said second set of primers amplifying a portion of the nucleic acid molecule of (a) comprising the hypervariable CDR3 region, wherein each of the second reverse primers includes: (i) a sequence that hybridizes the constant segment of the T cell receptor chain, (ii) a unique barcode, and (iii) a sequence identifying the chain of the T cell receptor. In certain aspects, the collection of second reverse primers includes the sequences: CGACTCAAGTGTGTGGXXXXXXGGGTCAGGGTTCTGGATAT (SEQ ID NO:744) and CGACTCAGATTGGTACXXXXXXACACSTTKTTCAGGTCCTC (SEQ ID NO:745); or CGACTCAAGTGTGTGGXXXXXXTTCTGGGTTCTGGATGT (SEQ ID NO:746) and CGACTCAGATTGGTACXXXXXXAGTCACATTTCTCAGATCCT (SEQ ID NO:747). In particular aspects, the collection of second reverse primers includes the sequences SEQ ID NOs:360-455 and SEQ ID NOs:456-551; or SEQ ID NOs:552-647 and SEQ ID NOs:648-743. In optional aspects, the kit further includes Cellular Indexing of the Transcriptome and Epitopes by Sequencing (CITE-Seq) primers, e.g., CITE-Seq primers having the sequences SEQ ID NO:356 and SEQ ID NO:357; or SEQ ID NO:358 and SEQ ID NO:359.

A method for analyzing a T cell receptor (e.g., TCRαβ or TCRγδ) of a single T cell is also provided, which includes the steps of (a) sorting single T cells from a sample into separate locations; (b) amplifying nucleic acid molecules encoding chains of the T cell receptor from one or more single T cells using the first set of primers from the kit to produce a first set of amplicon products in one or more locations of the separate locations; (c) performing nested polymerase chain reaction (PCR) on the amplified nucleic acid molecules encoding the chains of the T cell receptor in the first set of amplicon products with the second set of primers from the kit to produce a second set of amplicon products; and (d) sequencing the amplicon products. In some aspects, the nested PCR step (c) is performed in a multiwell plate, wherein each well of the multiwell plate comprises a unique barcode. In other aspects, the step of (d) sequencing the amplicon products includes ligating sequencing adapters onto the second set of amplicon products and sequencing the amplicon products by next generation sequencing.

### BRIEF DESCRIPTION OF THE DRAWINGS

FIG. 1 shows a schematic of the method of this invention. Notably, C-segment specific primers containing well-specific barcodes are introduced by PCR in Step II.

FIG. 2 depicts an experimental workflow overview, wherein T cells are stained with DNA-barcoded antibodies and/or MHC-multimers and are single cell sorted into 96 or 384 well plates.

FIG. 3 shows TCRαβ sequencing results of T cells from immunized mouse sorted into a 384-well plate. Diagram shows whether a TCRβ or TCRα chain was detected in each well.

### DETAILED DESCRIPTION OF THE INVENTION

A multiplex panel of primer sequences designed to amplify TCRαβ and TCRγδ sequences from single cells in an unbiased manner has now been developed. These primer sequences allow for the TCR repertoire to be analyzed using conventional next generation sequencing-based platforms in a highly efficient manner. Indeed, the instant single-cell TCR amplification and sequencing strategy offers a highly transparent, cell number agnostic approach for processing a plurality of cells at a time. Accordingly, this invention provides a kit and method for amplifying and analyzing TCR sequences from single T cells using a multiplex panel of oligonucleotide sequences designed to amplify TCR sequences in an unbiased manner. Using the kit and method of this invention, paired TCR chain sequences can be isolated at the single cell level in response to a variety of immune responses such as viral infections, tumors, and autoimmune patients thereby allowing for the design of effective immune cell-based therapies.

The present disclosure provides kits containing oligonucleotide primers and methods for analyzing nucleic acids encoding TCRs from individual T cells by high-throughput multiplex amplification and sequencing of the nucleic acids encoding the TCRs. As is conventional in the art, T cell receptors or TCRs are composed two chains, an a and β chain or an γ and δ chain and are respectively designated TCRαβ and TCRγδ. TCRs, like immunoglobulins, are composed of regions which arrange during T cell ontogeny. In genomic DNA, each TCR gene has V, J, and C regions; TCR β and δ polypeptides also have D regions. The V (variable), D (diversity), J (junctional) and C (constant) regions are separated from one another by spacer regions in the DNA. The sequence encoded by the V(D)J junction is called complementarity determining region 3 or CDR3. This sequence has the highest variability in both chains and determines the ability of a T cell to recognize an antigen peptide presented by the MHC molecule. The combinatorial variability is further increased by the subsequent heterodimeric pairing of chains. Accordingly, TCRs are generated which differ in their amino-terminal, or N-terminal, domains (called variable, or V regions, constructed from combinations of V, D, and J gene segments) but are the same elsewhere, including their carboxy-terminal, or C-terminal domains (called constant or C-segment).

As used herein, the term “primer” or “oligonucleotide primer” refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation step is typically achieved by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

The method of this invention generally involves sorting of single T cells into separate locations (e.g., separate wells of a multiwell titer plate) followed by nested polymerase chain reaction (PCR) amplification of nucleic acids encoding TCRs using the primers disclosed herein. The amplicons are barcoded to identify their cell of origin and analyzed by deep sequencing. Using the kit and method of the present disclosure, TCRs from individual T cells can be reconstituted for functional studies, ligand discovery, and/or screening therapeutics.

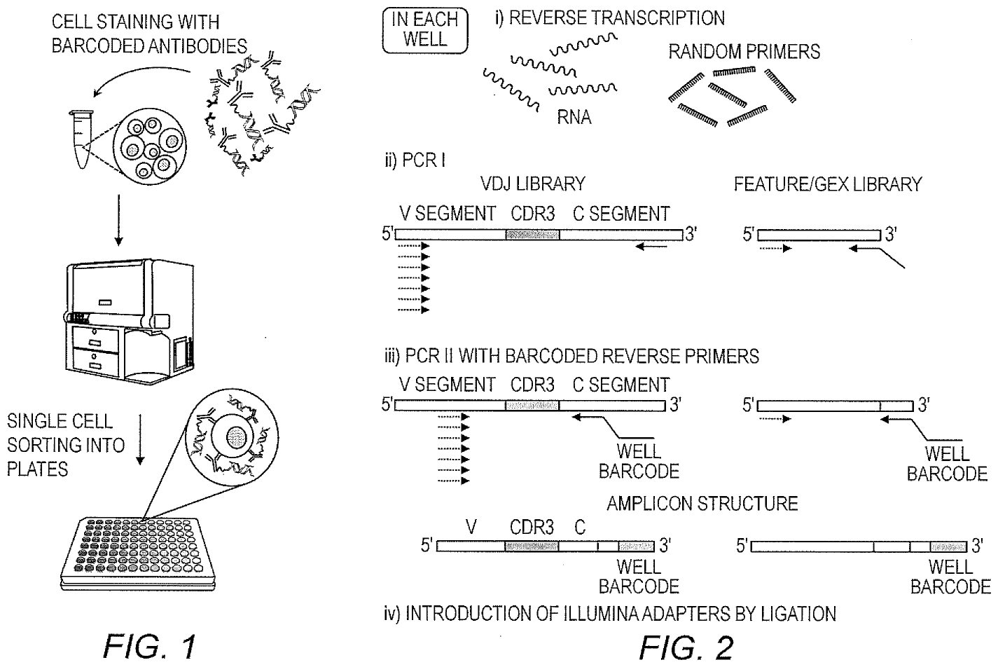

More specifically, this invention provides a kit and method for analyzing nucleic acid molecules encoding TCRs from individual T cells by sorting single T cells from a sample including a plurality of T cells into separate locations; amplifying nucleic acid molecules encoding chains of the T cell receptor from one or more single T cells using a first set of external primers to produce a first set of amplicon products in one or more locations of the separate locations; performing nested polymerase chain reaction (PCR) on the amplified nucleic acid molecules encoding the chains of the T cell receptor in the first set of amplicon products with the unique forward and reverse nested primers of this disclosure to produce a second set of amplicon products, wherein the reverse nested primer includes a barcode sequence; and sequencing the second set of amplicon products (FIG. 1), e.g., via a third round of PCR and next generation sequencing. Using the method of this invention, a wide variety of diseases, including inflammatory disorders, autoimmune diseases, infectious diseases, and cancer can be diagnosed and treated.

In carrying out the method of this invention, a biological sample including T cells is collected from a subject. The biological sample can be any sample of bodily fluid or tissue containing T cells, including but not limited to, samples of blood, thymus, spleen, lymph nodes, bone marrow, a tumor biopsy, or an inflammatory lesion biopsy. In particular, samples of T cells may be taken from sites of inflamed, infected, or injured tissue, including but not limited to sites of tumors, transplant rejection, tissue damage, such as caused by traumatic injury or autoimmune disease, and organs or tissues targeted by pathogenic organisms. The biological sample may also include samples from in vitro cell culture resulting from the growth of T cells from the subject in culture. The biological sample can be obtained from a subject by conventional techniques. For example, blood can be obtained by venipuncture. Surgical techniques for obtaining solid tissue samples are well known in the art. Samples may be obtained from a subject prior to diagnosis and throughout a course of treatment.

Subsequently, single T cells are isolated from the biological sample and sorted into separate locations. The separate locations can be separate reaction containers, such as wells of a multiwell plate (e.g., a 96-well plate, 384-well plate, 1536-well plate) or microwell array, capillaries, or tubes (e.g., 0.2 mL tubes, 0.5 mL tubes, 1.5 mL tubes), or chambers in a microfluidic device. Alternatively, the separate locations can be emulsion droplets that spatially separate cells.

Various methods are known in the art for isolating single cells. In some aspects, the sample is sorted to obtain single T cells using a flow cytometer. Methods of preparing a sample of cells for flow cytometry analysis is described in, e.g., U.S. Pat. Nos. 5,378,633; 5,631,165; 6,524,858; 5,266,269; 5,017,497; 6,549,876; US 2012/0178098; US 2008/0153170; US 2001/0006787; US 2008/0158561; US 2010/0151472; US 2010/0099074; US 2010/0009364; US 2009/0269800; US 2008/0241820; US 2008/0182262; US 2007/0196870; US 2008/0268494; WO 99/54494; Brown et al. (2000) Clin. Chem. 46:1221-9; McCoy et al. (2002) Hematol. Oncol. Clin. North Am. 16:229-43; and Scheffold (2000) J. Clin. Immunol. 20:400-7.

In some instances, single T cells can be isolated from a biological sample by appropriate dilution of a sample to allow distribution of a single cell in a small isolation volume to a separate location. In certain aspects, a microfluidic device is used for isolating single cells and distributing single cells to separate locations in the device, such as separate wells or chambers. Alternatively, a microfluidic device can be used to generate emulsion droplets containing single cells. For a description of techniques for isolating single cells and microfluidic devices for sorting single cells, see e.g., Huang et al. (2014) Lab Chip. 14(7):1230-1245; Zare et al. (2010) Annu. Rev. Biomed. Eng. 12:187-201; Novak et al. (2011) Angew. Chem. Int. Ed. 50:390-395; US 2010/0255471; US 2010/0285975; US 2010/0021984; US 2010/0173394; WO 2009/145925; and US 2009/0181859.

“Microfluidics device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, and the like. Microfluidics devices may further include valves, pumps, and specialized functional coatings on interior walls, e.g., to prevent adsorption of sample components or reactants, facilitate reagent movement by electroosmosis, or the like. Such devices are usually fabricated in or as a solid substrate, which may be glass, plastic, or other solid polymeric materials, and typically have a planar format for ease of detecting and monitoring sample and reagent movement, especially via optical or electrochemical methods. Features of a microfluidic device usually have cross-sectional dimensions of less than a few hundred square micrometers and passages typically have capillary dimensions, e.g., having maximal cross-sectional dimensions of from about 500 μm to about 0.1 μm. Microfluidics devices typically have, volume capacities in the range of from 1 μL to a few nL, e.g., 10 nL to 100 nL. The fabrication and operation of microfluidics devices are well-known in the art as described in U.S. Pat. Nos. 6,001,229; 5,858,195; 6,010,607; 6,033,546; 5,126,022; 6,054,034; 6,613,525; 6,399,952; WO 02/24322; WO 99/19717; and U.S. Pat. Nos. 5,587,128; 5,498,392.

In certain aspects, the sample is labeled with one or more detectable labels that bind to cells within the sample before sorting the cells. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. In some cases, the detectable label is linked to a binding agent that binds to a binding partner on a T cell in the sample. In case of labeling T cells, the binding agent may be an antibody (e.g., anti-CD3, anti-CD4, anti-CD8, anti-αβTCR, anti-CD14, anti-CD25, anti-CD45RA, anti-CD45RO, anti-FOXP3, etc.) or major histocompatibility complex (MHC) tetramer that specifically binds to a binding partner on or in a T cell. Thus, in some cases, the T cell is permeabilized before labeling. In some aspects, one or more detectable labels is used to classify a cell, e.g., T cell, within a sample, based on the amount of label bound to the cell.

In some aspects, a subset of cells within a sample is sorted as single cells into separate locations. Thus, cells may be sorted to include a first subset and exclude a second subset of cells within the sample. The first subset and second subset may be defined by a number of factors, including, but not limited to, amount of detectable label that is bound, size, light scattering properties, amount of staining by dyes that indicate viability or lack thereof, etc., of a cell. Thus, in some aspects, a T cell that is labeled with an anti-CD8, anti-CD14, MHC tetramer or a combination thereof, and is not labeled as being dead, is included to be sorted to generate single T cells in separate locations.

In some cases, sorting the T cells into separate locations as single cells may result in a subset of the separate locations having two or more T cells. These locations with potentially more than one T cells may be identified and flagged during data analysis of the sequencing data, and data from such locations in some cases may be removed from further analysis.

Once sorted, the nucleic acids encoding the T cell receptor chains in each T cell are analyzed using the primers described herein in polymerase chain reaction (PCR)-based techniques. As is conventional in the art, “polymerase chain reaction,” or “PCR” means a reaction for the in vitro amplification of specific nucleic acid sequences by the simultaneous primer extension of complementary strands of DNA. In PCR, a pair of primers is used in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well-known in the art and has been described in, e.g., Innis et al. (eds.) (1990) PCR Protocols, Academic Press, NY; Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195; 4,683,202; and 4,889,818.

In particular, PCR uses relatively short oligonucleotide primers that flank the target nucleotide sequence to be amplified, oriented such that their 3′ ends face each other, each primer extending toward the other. The polynucleotide sample is extracted and denatured, e.g., by heat, and hybridized with first and second primers that are present in molar excess. Polymerization is catalyzed in the presence of the four deoxyribonucleotide triphosphates (dNTPs: dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Tag), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. In some cases, PCR is carried out with a commercially available thermal cycler, e.g., Perkin Elmer.

Messenger RNA (mRNA) encoding TCR chains may be amplified by reverse transcribing the RNA into cDNA, and then performing PCR. This type of amplification, referred to as “reverse transcription PCR,” or “RT-PCR,” is well-known in the art and described, e.g., in U.S. Pat. No. 5,168,038, which is incorporated herein by reference. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84. Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNAse H activity, such as AMV reverse transcriptase. In some cases, exogenous RNAse H, such as E. coli RNAse H, is added, even when AMV reverse transcriptase is used. RNAse H is readily available from, e.g., Bethesda Research Laboratories. Other suitable reverse transcriptases include transcriptases sold under the tradenames SUPERSCRIPT® II Reverse Transcriptase (ThermoFisher) and PROTOSCRIPT® II Reverse Transcriptase (New England Biolabs). The RNA transcripts produced using these enzymes and methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

The method of the present disclosure uses a multiplexed nested PCR approach. “Nested PCR” refers to PCR that is carried out in at least two steps, wherein the amplicon product from a first round of PCR becomes the template for a second round of PCR using a second set of primers, at least one of which binds to an interior location of the amplicon from the first round of PCR, to generate a second amplicon product. In certain aspects, a third round of PCR is carried out on the second amplicon product using a third set of primers to generate a third amplicon product, which is sequenced.

In certain aspects, the nested PCR is multiplexed, wherein the nested PCR is carried out with T cell target sequences encoding TCRs simultaneously in the same reaction mixture. See, e.g., Bernard et al. (1999) Anal. Biochem. 273:221-228. Distinct sets of primers are employed for each sequence being amplified as described herein. Exemplary primers are provided in Tables 1-18 and 23-26 for amplifying human, mouse, and macaque TCRs (e.g., both α and β or γ and δ chains of the heterodimer), and also for introducing well-specific barcodes and chain specific sequences. Changes to the nucleotide sequences of these primers may be introduced corresponding to genetic variations in particular T cells. For example, up to three nucleotide changes, including 1 nucleotide change, 2 nucleotide changes, or three nucleotide changes, may be made in a sequence selected from the group of SEQ ID NOs: 1-307 and/or SEQ ID NO:744-747, wherein the oligonucleotide primer is capable of hybridizing to and amplifying or sequencing a T cell target nucleic acid (i.e., nucleic acids encoding TCRαβ or TCRγδ).

In certain cases, a first set of primers used to amplify a target nucleic acid, i.e., a nucleic acid encoding TCRαβ or TCRγδ, may contain a primer that specifically hybridizes to and amplifies, when paired with another appropriate primer in the first set, the target nucleic acid during a first round of PCR. A second set of primers may then be used to further amplify the target nucleic acid when the second set contains a primer that specifically hybridizes to and amplifies, when paired with another appropriate primer in the second set, a specific amplification product of the first round of PCR during a second round of PCR. Similarly, a third set of primers may then be used to further amplify the target nucleic acid when the third set contains a primer that specifically hybridizes to and amplifies, when paired with another appropriate primer in the third set, a specific amplification product of the second round of PCR during a third round of PCR.

In some aspects, primers within a set of primers may include, in addition to a sequence that hybridizes to a target nucleic acid, or an amplification product thereof, a common sequence and/or a barcode sequence. The common sequence may be the same sequence among a plurality of primers that otherwise hybridize to and amplify, when appropriately paired with another primer, different target nucleic acids, or amplification products thereof. In some aspects, the common sequence in a primer used during a round of PCR enables a primer used during a following round of PCR to anneal to and amplify, when paired with an appropriate primer, the target nucleic acid by serving as an annealing site for the primer used during a following round of PCR. As such, in some cases, the common sequence in a primer used during a round of PCR is a sequence that does not hybridize to target-specific sequences of a target nucleic acid, or to a specific amplification product from a previous round of PCR. In some cases, the common sequence is a sequence that hybridizes to a target nucleic acid, if, for example, the target nucleic acid includes a sequence that is shared among different target nucleic acids, e.g., a sequence encoding a constant region of a TCR.

The multiplexed PCR reactions may be carried out in one or more of the separate locations into which single T cells from a sample have been sorted. Ideally, the amplification products of the multiplexed PCR reaction, which are in multiple separate locations, are combined into one pool before sequencing. In certain aspects, the barcode sequence used in one of the rounds of the multiplexed PCR reactions may be used to enable identification of the location, e.g., well, from which a particular sequenced amplification product originated, as described further herein.

The present disclosure provides sets of primers that amplify nucleic acid molecules encoding T cell receptors, in particular all or a portion of the T cell receptor chains that include the hypervariable CDR3 region. In general, the forward primers of the first and second collection of primers hybridize to conserved sequences encoding the V segment of each T cell receptor chain and the reverse primers hybridize to nucleic acids encoding the C segment of each T cell receptor chain. Accordingly, in the first and second collection of primers, the forward provide for multiple distinct T cell receptor variable region sequences. See Tables 1-18. By comparison, a single reverse primer is used for each TCR chain in the first set of primers, i.e., a single primer that hybridizes to nucleic acids encoding the constant segment of the α, β, γ and δ chains. See Tables 1-4, 9-12 and 17. A key aspect of this invention is provided in the second set of reverse primers, which is a collection of primers that each share the same sequence, which hybridizes to the constant segment of the T cell receptor chain, as well as a unique well-specific barcode corresponding to a well in a multiwell plate, and a sequence identifying the chain of the T cell receptor, i.e., a sequence specific for α chains, β chains, γ chains and δ chains. In certain aspects, the kit and method include the use of second reverse primers having the sequences:
(i) CGACTCAAGTGTGTGGXXXXXXGGGTCAGGGTTCTGGATAT (SEQ ID NO:744) and CGACTCAGATTGGTACXXXXXXACACSTTKTTCAGGTCCTC (SEQ ID NO:745) for nested amplification of human TRC α and β chains, respectively; or
(ii) CGACTCAAGTGTGTGGXXXXXXTTCTGGGTTCTGGATGT (SEQ ID NO:746) and CGACTCAGATTGGTACXXXXXXAGTCACATTTCTCAGATCCT (SEQ ID NO:747) for nested amplification of mouse TRC α and β chains, respectively. In accordance with this aspect, the 5′ end of the primers includes a sequence for identifying a second amplicon as an α chain (i.e., CGACTCAAGTGTGTGG (SEQ ID NO:748) or β chain (i.e., CGACTCAGATTGGTAC (SEQ ID NO:749) of the T cell receptor; the middle of the primers includes a unique well-specific barcode (i.e., XXXXXX); and the 3′ end of the primers includes a sequence that hybridizes the constant segment of the cell T receptor chain (i.e., GGGTCAGGGTTCTGGATAT (SEQ ID NO:750) for human TRCα, ACACSTTKTTCAGGTCCTC (SEQ ID NO:751) for human TRCβ, TTCTGGGTTCTGGATGT (SEQ ID NO:752) for mouse TRCα, and AGTCACATTTCTCAGATCCT (SEQ ID NO:753) for mouse TRCβ). Exemplary collections of second reverse primers are provided in Tables 23-26.

In aspects pertaining to amplification of nucleic acid molecules encoding at least a portion of human α and β T cell receptor chains from single T cells, a first set of primers includes a first set of forward primers set forth in SEQ ID NOs:1-40 and SEQ ID NOs:42-70, or a variant thereof that differs by up to three nucleotides, and a first set of reverse primers set forth in SEQ ID NOs:41 and 71, or a variant thereof that differs by up to three nucleotides. In aspects pertaining to amplification of nucleic acid molecules encoding at least a portion of human γ and δ T cell receptor chains from single T cells, a first set of primers includes a first set of forward primers set forth in SEQ ID NOs:72-80 and SEQ ID NOs:82-89, or a variant thereof that differs by up to three nucleotides, and a first set of reverse primers set forth in SEQ ID NOs:81 and 90, or a variant thereof that differs by up to three nucleotides. In aspects pertaining to amplification of nucleic acid molecules encoding at least a portion of mouse α and β T cell receptor chains from single T cells, a first set of primers includes a first set of forward primers set forth in SEQ ID NOs:181-203 and SEQ ID NOs:205-223, or a variant thereof that differs by up to three nucleotides, and a first set of reverse primers set forth in SEQ ID NOs:204 and 224, or a variant thereof that differs by up to three nucleotides. In aspects pertaining to amplification of nucleic acid molecules encoding at least a portion of mouse γ and δ T cell receptor chains from single T cells, a first set of primers includes a first set of forward primers set forth in SEQ ID NOs:225-229 and SEQ ID NOs:231-243, or a variant thereof that differs by up to three nucleotides, and a first set of reverse primers set forth in SEQ ID NOs:230 and 244, or a variant thereof that differs by up to three nucleotides. In aspects pertaining to amplification of nucleic acid molecules encoding at least a portion of macaque α and β T cell receptor chains from single T cells, a first set of primers includes a first set of forward primers set forth in SEQ ID NOs:2, 4, 5, 13-16, 18, 20, 24, 25, 29, 30, 33, 34, 310-329 and SEQ ID NOs:42-70, or a variant thereof that differs by up to three nucleotides, and a first set of reverse primers set forth in SEQ ID NOs:330 and SEQ ID NO:71, or a variant thereof that differs by up to three nucleotides. The T cell receptors amplified with the first set of primers include the T cell receptor a chain and T cell receptor β chain, or T cell receptor γ chain and T cell receptor δ chain.

The present disclosure further provides a second set of nested primers that amplify the first set of amplicons. In aspects pertaining to amplification of nucleic acid molecules encoding human α and β T cell receptor chains, a second set of nested primers includes a second set of forward nested primers set forth in SEQ ID NOs:91-130 and SEQ ID NOs:132-160, or a variant thereof that differs by up to three nucleotides, and a second set of reverse nested primers set forth in SEQ ID NOs:360-455 and SEQ ID NOs:456-551, or a variant thereof that differs by up to three nucleotides, wherein the second set of nested primers amplify the first set of amplicons to produce a second set of amplicons. In aspects pertaining to amplification of nucleic acid molecules encoding mouse α and β T cell receptor chains, a second set of nested primers includes a second set of forward nested primers set forth in SEQ ID NOs:245-267 and SEQ ID NOs:269-287, or a variant thereof that differs by up to three nucleotides, and a second set of reverse nested primers set forth in SEQ ID NOs:552-647 and SEQ ID NOs:648-743, or a variant thereof that differs by up to three nucleotides, wherein the second set of nested primers amplify the first set of amplicons to produce a second set of amplicons

Advantageously, barcode sequences are included in the second set of reverse nested primers to identify the well and/or T cell from which each amplified nucleic acid originated. The use of barcodes allows nucleic acid analytes from different cells to be pooled in a single reaction mixture for sequencing while still being able to trace back a particular target nucleic acid to the particular cell from which it originated. Each cell is identified by a unique barcode sequence comprising at least five to six nucleotides. In accordance with this invention, barcode sequences are added during amplification by carrying out PCR with a primer that contains a region include the barcode sequence and a region that is complementary to the target nucleic acid of interest such that the barcode is incorporated into the final amplified nucleic acid product. Exemplary well-specific barcode sequences are provided in Tables 23-26. Exemplary primers for introducing barcodes into an amplicon are provided in Tables 23-26. In one aspect, a primer for introducing a barcode sequence into an amplicon of a nucleic acid encoding a TCR has a sequence set forth in SEQ ID NOs:360-455 and SEQ ID NOs:456-551; or SEQ ID NOs:552-647 and SEQ ID NOs:648-743.

The primers of this invention can be readily synthesized by standard techniques, e.g., solid phase synthesis via phosphoramidite chemistry, as disclosed in U.S. Pat. Nos. 4,458,066; 4,415,732; Beaucage et al. (1992) Tetrahedron 48:2223-2311. Other chemical synthesis methods include, for example, the phosphotriester method described by Narang et al. (1979) Meth. Enzymol. 68:90 and the phosphodiester method disclosed by Brown et al. (1979) Meth. Enzymol. 68:109. Poly(A) or poly(C), or other non-complementary nucleotide extensions may be incorporated into oligonucleotides using these same methods. Hexaethylene oxide extensions may be coupled to the oligonucleotides by methods known in the art. See, e.g., Cload et al. (1991) J. Am. Chem. Soc. 113:6324-6326; U.S. Pat. No. 4,914,210; Durand et al. (1990) Nucleic Acids Res. 18:6353-6359; and Horn et al. (1986) Tet. Lett. 27:4705-4708.

The primers of this invention are in the range of between 10-100 nucleotides in length, such as 15-40, 20-40, 15-70, 50-90 and so on, more typically in the range of between 15-90 nucleotides long, and any length between the stated ranges. In certain aspects, a primer oligonucleotide has a sequence selected from the group of SEQ ID NOs:1-130, 132-160, 162-170, 172-179, 181-267, 269-287, 289-293, 295-307, 360-743, or a fragment thereof including at least about 6 contiguous nucleotides, at least about 8 contiguous nucleotides, at least about 10-12 contiguous nucleotides, or at least about 15-20 contiguous nucleotides; or a variant thereof with a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto. Changes to the nucleotide sequences of SEQ ID NOS:1-130, 132-160, 162-170, 172-179, 181-267, 269-287, 289-293, 295-307, 360-743 may be introduced corresponding to genetic variations in particular T cells. In certain aspects, up to three nucleotide changes, including 1 nucleotide change, 2 nucleotide changes, or three nucleotide changes, may be made in a sequence selected from the group of SEQ ID NOs:1-130, 132-160, 162-170, 172-179, 181-267, 269-287, 289-293, 295-307, 360-743, wherein the oligonucleotide primer is capable of hybridizing to and amplifying a particular T cell receptor target nucleic acid.

Moreover, the oligonucleotides, particularly the primer oligonucleotides for amplification or sequencing, may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al. (1978) Nucl. Acids Res. 5:363-384, which discloses the use of ferritin-avidin-biotin labels; and Chollet et al. (1985) Nucl. Acids Res. 13:1529-1541, which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139; Gibson et al. (1987) Nucl. Acids Res. 15:6455-6467 and U.S. Pat. No. 4,605,735. Methods are also available for synthesizing sulfhydryl-derivatized oligonucleotides, which can be reacted with thiol-specific labels, see, e.g., U.S. Pat. No. 4,757,141; Connolly et al. (1985) Nucl. Acids Res. 13:4485-4502 and Spoat et al. (1987) Nucl. Acids Res. 15:4837-4848. A comprehensive review of methodologies for labeling DNA fragments is provided in Matthews et al. (1988) Anal. Biochem. 169:1-25.

For example, oligonucleotides may be fluorescently labeled by linking a fluorescent molecule to the non-ligating terminus of the molecule. Guidance for selecting appropriate fluorescent labels can be found in Smith et al. (1987) Meth. Enzymol. 155:260-301; Karger et al. (1991) Nucl. Acids Res. 19:4955-4962; and Guo et al. (2012) Anal. Bioanal. Chem. 402(10):3115-3125. Fluorescent labels include fluorescein and derivatives thereof, such as disclosed in U.S. Pat. No. 4,318,846 and Lee et al. (1989) Cytometry 10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional dyes include Yakima Yellow, Texas Red, 3-(ε-carboxypentyl)-3′-ethyl-5,5′-dimethyloxa-carbocyanine (CYA), 6-carboxy fluorescein (FAM), 5,6-carboxyrhodamine-110 (R110), 6-carboxyrhodamine-6G (R6G), N′,N′,N′,N′-tetramethyl-6-carboxyrhodamine (TAMRA), 6-carboxy-X-rhodamine (ROX),2′,4′,5′,7′,-tetrachloro-4-7-dichlorofluorescein (TET),2′,7′-dimethoxy-4′,5′-6 carboxyrhodamine (JOE), 6-carboxy-2′,4,4′,5′,7,7′-hexachlorofluorescein (HEX), Dragonfly orange, ATTO-Tec, Bodipy, and VIC, and dyes available under the trademarks SYBR® green, SYBR® gold, CAL FLUOR® Orange 560, CAL FLUOR® Red, QUASAR® Blue 670, ALEXA®, Cy3®, and Cy5®. These dyes are commercially available from various suppliers such as Life Technologies (Carlsbad, CA), Biosearch Technologies (Novato, CA), and Integrated DNA Technologies (Coralville, IA). Fluorescent labels include fluorescein and derivatives thereof, such as disclosed in U.S. Pat. No. 4,318,846 and Lee et al. (1989) Cytometry 10:151-164, and 6-FAM, JOE, TAMRA, ROX, HEX-1, HEX-2, ZOE, TET-1, or NAN-2, and the like.

Oligonucleotides can also be labeled with a minor groove binding (MGB) molecule, such as disclosed in U.S. Pat. Nos. 6,884,584; 5,801,155; Afonina et al. (2002) Biotechniques 32:940-944, 946-949; Lopez-Andreo et al. (2005) Anal. Biochem. 339:73-82; and Belousov et al. (2004) Hum. Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J (ed) Academic Press, San Diego, CA; Nelson et al. (1994) “Application of the Hybridization Protection Assay (HPA) to PCR” in The Polymerase Chain Reaction, Mullis et al. (eds.) Birkhauser, Boston, MA; Weeks et al. (1983) Clin. Chem. 29:1474-1479; Berry et al. (1988) Clin. Chem. 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

T cells may be pre-treated in any number of ways prior to amplification and sequencing of nucleic acids. For instance, in certain aspects, the T cell may be treated to disrupt (or lyse) the cell membrane, for example by treating the samples with one or more detergents and/or denaturing agents (e.g., guanidinium agents). Nucleic acids may also be extracted from samples, for example, after detergent treatment and/or denaturing as described above. Total nucleic acid extraction may be performed using known techniques, for example by non-specific binding to a solid phase (e.g., silica). See, e.g., U.S. Pat. Nos. 5,234,809; 6,849,431; 6,838,243; 6,815,541; and 6,720,166.

In certain aspects, the target nucleic acids are separated from non-homologous nucleic acids using capture oligonucleotides immobilized on a solid support. Such capture oligonucleotides contain nucleic acid sequences that are complementary to a nucleic acid sequence present in the target T cell nucleic acid analyte such that the capture oligonucleotide can “capture” the target nucleic acid. Capture oligonucleotides can be used alone or in combination to capture T cell nucleic acids. For example, multiple capture oligonucleotides can be used in combination, e.g., 2, 3, 4, 5, 6, etc. different capture oligonucleotides can be attached to a solid support to capture target T cell nucleic acids. In certain aspects, one or more capture oligonucleotides can be used to bind T cell target nucleic acids either prior to or after amplification by primer oligonucleotides and/or sequencing.

As T cells may be sorted into single T cells in separate locations, e.g., separate wells, in the present method, as described above, some aspects of the present disclosure include a composition including one or more sets of forward and reverse primers and/or sets of primer pairs, as described above, and nucleic acids from a single T cell. After single T cells are sorted to separate locations, they may be lysed in order to release cellular contents, such as nucleic acids (e.g., mRNA, miRNA, chromosomal DNA, mitochondrial DNA, etc.). The released nucleic acids may then provide templates, including any target nucleic acids, from which PCR may be carried out using the primer compositions of the present disclosure. A composition that contains nucleic acids from a single T cell may be distinguished from a composition that contains nucleic acids from two or more T cells by, e.g., determining the number of one or more autosomal loci of chromosomal DNA using sequencing or other suitable methods, as described in, e.g., Kalisky et al. (2011) Nat. Methods 8:311; Fu et al. (2011) Proc. Natl. Acad. Sci. USA 108:9026; and Shuga et al. (2013) Nucleic Acids Res. 41:e159. Thus, in some aspects, the composition contains one or more sets of forward and reverse primers and/or sets of primer pairs, as described above, and T cell nucleic acids from less than two T cells. In some aspects, the composition contains no nucleases and/or contains nuclease inhibitors and/or provides buffering conditions that inhibits or reduces nucleic acid degradation at least until the first round of amplification.

Advantageously, the primers of the instant kit and method do not include sequencing adapters. Rather, sequencing adapters are ligated to the second set of amplicon products prior to sequencing. In this respect, additional sequences can be added to primers disclosed herein without hindering subsequent sequence analysis. Moreover, introduction of sequence adapters by ligation allows for amplification of all nucleic acids in a well of interest so that sequence information of all nucleic acids in the well is obtained. In particular, the instant method can be used in conjunction with transcriptome analysis methods such as Cellular Indexing of Transcriptomes and Epitopes by Sequencing (CITE-seq). Thus, in some aspects, the kit and method of this invention further include CITE-Seq primers. As is known in the art, CITE-seq uses DNA-barcoded antibodies (e.g., TotalSeqC DNA-barcoded antibodies commercially available from Biolegend) or MHC-multimers (e.g., Immudex) to convert detection of proteins into a quantitative sequence-based readout. See Stoeckius et al. (2017) Nat Methods 14:865-868 and US 2018/0251825 A1, the latter of which is incorporated herein by reference in its entirety. The incorporation of CITE-Seq primers in the instant method and kit allows for simultaneous detection of paired TCR, surface protein expression, and cognate epitope in the same well from a single T cell (See FIG. 2). In certain aspects, the CITE-Seq primers used in the methods and kits of this invention have the sequences SEQ ID NO:356 and SEQ ID NO:357; or SEQ ID NO:358 and SEQ ID NO:359.

To sequence the nucleic acids encoding TCRs, as well as other nucleic acid molecules in the multiwell plates (e.g., transcriptome analysis), adapter sequences are added to amplicons to facilitate high-throughput amplification or sequencing. For example, a pair of adapter sequences are added at the 5′ and 3′ ends of a nucleic acid molecule to allow amplification and/or sequencing of multiple nucleic acid molecule simultaneously by the same set of primers. Exemplary adapter sequences include the sequences: TCGTCGGCAGCGTCAGATGTGTATAAGAGACAG (SEQ ID NO:754) and GTCTCGTGGGCTCGGAGATGTGTATAAGAGACAG (SEQ ID NO:755). See ILLUMINA®, 16S Metagenomic Sequencing Library Preparation, Preparing 16S Ribosomal RNA Gene Amplicons for the Illumina MiSeq System, Part #15044223 Rev. B.

Any high-throughput technique for sequencing can be used in the practice of the invention. DNA sequencing techniques include sequencing by synthesis using reversibly terminated labeled nucleotides, pyrosequencing, 454 sequencing, sequencing by synthesis using allele specific hybridization to a library of labeled clones followed by ligation, real-time monitoring of the incorporation of labeled nucleotides during a polymerization step, polony sequencing, SOLID sequencing, and the like. These sequencing approaches can thus be used to sequence target TCR nucleic acids of amplified from single T cells.

Certain high-throughput methods of sequencing include a step in which individual molecules are spatially isolated on a solid surface where they are sequenced in parallel. Such solid surfaces may include nonporous surfaces (such as in Solexa sequencing, e.g., Bentley et al. (2008) Nature 456:53-59; or Complete Genomics sequencing, e.g., Drmanac et al. (2010) Science 327:78-81), arrays of wells, which may include bead- or particle-bound templates (such as with 454, e.g. Margulies et al. (2005) Nature 437:376-380; or Ion Torrent sequencing, e.g., US 2010/0137143 or US 2010/0304982), micromachined membranes (such as with SMRT sequencing, e.g., Eid et al. (2009) Science 323:133-138), or bead arrays (as with SOLiD sequencing or polony sequencing, e.g., Kim et al. (2007) Science 316:1481-1414). Such methods may include amplifying the isolated molecules either before or after they are spatially isolated on a solid surface. Prior amplification may include emulsion-based amplification, such as emulsion PCR, or rolling circle amplification. In certain aspects, amplification is carried out using a third set of primers in a third round of PCR with the second amplicon product as a template, i.e., for paired-end sequencing. Of particular interest is sequencing on the ILLUMINA® MISEQ® platform, which uses reversible-terminator sequencing by synthesis technology (see, e.g., Shen et al. (2012) BMC Bioinformatics 13:160; et Junemann al. (2013) Nat. Biotechnol. 31(4):294-296; Glenn (2011) Mol. Ecol. Resour. 11(5):759-769; Thudi et al. (2012) Brief Funct. Genomics 11(1):3-11).

The present disclosure also provides for analyzing multiplexed single cell sequencing data, such as those acquired using the method of analyzing single T cells described herein. In one implementation, a user may access a file on a computer system, wherein the file is generated by sequencing multiplexed PCR amplification products from multiple single T cells by, e.g., a method of analyzing single T cells, as described herein. Thus, the file may include a plurality of sequencing reads for a plurality of nucleic acids derived from multiple T cells. Each of the sequencing reads may be a sequencing read of a nucleic acid that contains a target nucleic acid nucleotide sequence (i.e., a nucleotide sequence encoding T cell receptor) and one or more barcode sequences that identifies the single cell source (e.g., a single cell in a well in a multiwell plate, a capillary, a microfluidic chamber, etc.) from which the nucleic acid originated (e.g., after multiple nested PCR of the target nucleic acid expressed by a single T cell in the well). In some aspects, the sequencing read is a paired-end sequencing read.

The sequencing reads in the file may be assembled to generate a consensus sequence of a target nucleic acid nucleotide sequence by matching the nucleotide sequence corresponding to the target nucleic acid sequence and the barcode sequences contained in each sequencing read. Those sequencing reads that originate from the same single cell source (e.g., same well) and have a target sequence that has a higher identity to a reference sequence than a threshold identity level may be assigned to the same target nucleic acid that was initially amplified from the single cell source and may be grouped into a subset representing the target nucleic acid. The number of sequencing reads within the subset indicates how likely it is that the consensus sequence assembled from the sequencing reads in a subset is part of an actual nucleic acid molecule that was present in the single cell source. Thus, if the number of sequencing reads in a subset is above a background level, the consensus sequence derived from the subset may be considered to represent an actual sequence of a target nucleic acid in the single cell source. The consensus sequence may then be outputted, e.g., to a display, printout, database, etc.

In some aspects, the reference sequence is a sequence for the target nucleic acid in a reference database, such as GENBANK®. Thus, in some aspects, a target sequence in a first sequencing read in a subset of sequencing reads, as described above, is 80% or more, e.g., 85% or more, 90% or more, 95% or more, or up to 100% identical to a reference sequence for the target nucleic acid from a reference database. In some aspects, the reference sequence is one or more other sequences in sequencing reads of the same subset. Thus, in such cases, a target nucleotide sequence in a first sequencing read in a subset of sequencing reads, as described above, is 80% or more, e.g., 85% or more, 90% or more, 95% or more, or up to 100% identical to a target nucleotide sequence in a second sequencing read in the same subset. In some instances, a target nucleotide sequence in a first sequencing read in a subset is 80% or more, e.g., 85% or more, 90% or more, 95% or more, or up to 100% identical to a target nucleotide sequence in all other sequencing reads in the same subset.

In certain aspects, the sequencing reads are generated by a method of analyzing a T cell as disclosed herein. As such, in some aspects, the target nucleic acid sequence contained in the sequenced nucleic acid is flanked on the 5′ and 3′ ends by a common sequence and a barcode sequence. The barcode sequence may contain a sequence that specifies the source of the target nucleic acid (e.g., the plate among a plurality of plates, the row among a plurality of rows in a multiwall plate, and/or the column among a plurality of columns in a multiwall plate, etc.). The common sequence and/or barcode sequence is incorporated into the amplified target nucleic acid during a round of the multiplex amplification process, e.g., during the second round of amplification, as described above, to provide for a primer annealing site that may be used in the next round amplification (e.g., third round amplification). Thus, the common sequences at the ends of the amplified target nucleotide sequence may be sequences exogenous to the target nucleic acid, are ideally different from one another, and may not be a sequence that can hybridize to the target nucleotide sequence before the second round of amplification. The length of the common sequences may be in the range of 17 to 30 nucleotides long, e.g., 18 to 28 nucleotides long, 19 to 26 nucleotides long, including 20 to 25 nucleotides long.

The output of the analysis may be provided in any convenient form. In some aspects, the output is provided on a user interface, a printout, in a database, etc. and the output may be in the form of a table, graph, raster plot, heat map etc. In some aspects, the output is further analyzed to determine properties of the single cell from which a target nucleotide sequence was derived. Further analysis may include correlating expression of a plurality of target nucleotide sequences within single cells, principal component analysis, clustering, statistical analyses, etc.

A computer system for implementing the present computer-implemented method may include any arrangement of components as is commonly used in the art. The computer system may include a memory, a processor, input and output devices, a network interface, storage devices, power sources, and the like. The memory or storage device may be configured to store instructions that enable the processor to implement the present computer-implemented method by processing and executing the instructions stored in the memory or storage device.

In certain alternative aspects, the present method of analyzing T cells includes stimulating T cells in a sample obtained from a subject before sorting single T cells into separate locations. The stimulating may be achieved by any convenient method. Stimulating T cells may include, but are not limited to, contacting the T cells with 12-myristate 13-acetate (PMA) and ionomycin, with PMA and anti-CD3/anti-CD28, with one or more antigens specifically recognized by one or more T cells of interest in the sample, or with extracts of cells or tissues. In some cases, a sample is divided into to a first sample whose T cells are stimulated and a second sample whose T cells are unstimulated, then the two samples are analyzed separately according to the method described herein.

In certain aspects, the present method of analyzing single T cells is an efficient method of analyzing nucleic acids expressed in single T cells. The presence of a T cell receptor may be detected by the present method in 70% or more, e.g., 80% or more, 85% or more, 90% or more, 92% or more, or 94% or more, and in some cases 100% or less, e.g., 95% or less, or 94% or less of the single T cells sorted into the separate locations. In some instances, the presence of a T cell receptor may be detected by the present method in a range of 70 to 100%, e.g., a range of 80 to 98%, a range of 85 to 95%, including a range of 90 to 94% of the single T cells sorted into the separate locations. In some aspects, presence of a T cell receptor alpha chain may be detected by the present method in 70% or more, e.g., 80% or more, 85% or more, or 90% or more, and in some cases 100% or less, e.g., 95% or less, or 90% or less of the single T cells sorted into the separate locations. In some instances, the presence of a T cell receptor alpha chain may be detected by the present method in a range of 70 to 100%, e.g., a range of 75 to 95%, a range of 80 to 92%, including a range of 85 to 90% of the single T cells sorted into the separate locations. In some aspects, presence of a T cell receptor beta chain may be detected by the present method in 85% or more, e.g., 90% or more, or 94% or more, and in some cases 100% or less, e.g., 97% or less, or 94% or less of the single T cells sorted into the separate locations. In some instances, the presence of a T cell receptor beta chain may be detected by the present method in a range of 85 to 100%, e.g., a range of 98 to 98%, a range of 90 to 96%, including a range of 91 to 95% of the single T cells sorted into the separate locations.

In certain aspects, the present method of analyzing single T cells is a sensitive method of analyzing nucleic acids expressed in single T cells. The present method may provide for detecting the presence of 50 molecules or less, e.g., 25 molecules or less, 20 molecules or less, 10 molecules or less, and down to 2 molecules of a target nucleic acid (e.g., mRNA for a T cell receptor) in a single T cell.

The technology described herein provides highly efficient TCR sequencing of single T cells and finds numerous applications in basic research and development. This methodology can be performed at reasonable cost by any standardly equipped laboratory with access to flow cytometry and deep sequencing. Sequencing TCRs of single T cells provides information about the ancestry of particular T cells. Furthermore, the sequences of nucleic acids amplified from T cells can be analyzed for splice variations, somatic mutations, or genetic polymorphisms. Of particular interest are genetic variations and mutations associated with immune disorders or cancer. In addition, the analysis described herein can be even further extended to obtain information about the phenotype of the single cells using Total-SeqC antibodies, thereby allowing for the identification of cross-reactive immune responses.

Additionally, knowledge of the sequences of TCRs from individual cells allows TCRs to be reconstituted for functional studies. For example, after analyzing a T cell as described herein and identifying a sequence encoding a TCRα polypeptide and a sequence encoding a TCRβ polypeptide from a single T cell, recombinant constructs expressing the TCRαβ heterodimer can be constructed. A host cell can be transformed with one or more recombinant polynucleotides encoding the TCR (e.g., separate monocistronic constructs expressing each polypeptide chain of the TCR heterodimer or a bicistronic construct expressing both the TCRα polypeptide and the TCR beta polypeptide). The TCR of the single cell can be produced by culturing the host cell under conditions suitable for the expression of the TCRα polypeptide and the TCRβ polypeptide and recovering the TCRαβ heterodimer from the host cell culture.

The reconstituted TCR can be used in screening to determine the target antigen bound by the TCR by contacting the TCR with potential target antigens displayed in complexes with major histocompatibility complex (MHC) and determining whether or not the target antigen binds to the TCR. The TCR can be screened for antigen binding in a high-throughput manner by providing a peptide library including a plurality of peptides displayed by major histocompatibility complex (MHC) molecules; and contacting the plurality of peptides with the TCR; and identifying at least one peptide-MHC complex that binds to the TCR. Any suitable antigen may find use in the present method. Exemplary antigens include, but are not limited to, antigenic molecules from infectious agents, auto-/self-antigens, tumor-/cancer-associated antigens, etc.

The present disclosure also provides kits for carrying out the method of the present disclosure. The above-described reagents, including the primers for amplification of target nucleic acid molecules encoding TCRs, and optionally other reagents for performing nucleic acid amplification (e.g., by RT-PCR) and/or sequencing can be provided in kits with suitable instructions and other necessary reagents for analyzing single T cells. The kit will normally contain in separate containers the primers and other reagents (e.g., polymerases, nucleoside triphosphates, and buffers). All primers within a set of primers may in some cases be provided in one container. In some cases, different subsets of primers within a set of primers may be provided in separate containers. Instructions (e.g., written, CD-ROM, DVD, flash drive, etc.) for carrying out the analysis of T cells usually will be included in the kit. The kit can also contain other packaged reagents and materials (i.e., wash buffers, cell lysis agents, reagents for extraction and purification of nucleic acids, and the like). Analysis of single T cells, as described herein, can be conducted using these kits.

Thus, the present disclosure provides kits that find use in performing the present method, as described above. In certain aspects, the kit includes (a) a first set of primers including a collection of first forward primers and a first reverse primer for each chain of the T cell receptor, said first set of primers amplifying a nucleic acid molecule encoding a portion of the T cell receptor including the hypervariable CDR3 region, wherein each first reverse primer hybridizes to a sequence encoding the constant segment of the T cell receptor chain; and (b) a second set of primers including a collection of second forward primers and a collection of second reverse primers for each chain of the T cell receptor, said second set of primers amplifying a portion of the nucleic acid molecule of (a) including the hypervariable CDR3 region, wherein each of the second reverse primers has a sequence that hybridizes the constant segment of the T cell receptor chain, a unique barcode, and a sequence identifying the chain of the T cell receptor. In certain aspects, the first set and second set of primers are included in separate containers. In other aspects, the first set of forward primers, first set of reverse primers, second set of forward primers, and second set of reverse primers are each in separate containers. In particular aspects, the collection of first forward primers have the nucleotide sequences of SEQ ID NOs:1-40 and SEQ ID NOs:42-70; SEQ ID NOs:72-80 and SEQ ID NOs:82-89; SEQ ID NOs:181-203 and SEQ ID NOs:205-223; or SEQ ID NOS 225-229 and SEQ ID NOs:231-243. In other aspects, the first reverse primer for each chain of the T cell receptor has the nucleotide sequences of SEQ ID NO:41 and SEQ ID NO:71; SEQ ID NO:81 and SEQ ID NO:90; SEQ ID NO:204 and SEQ ID NO:224; or SEQ ID NO:230 and SEQ ID NO:244. In further aspects, the collection of second forward primers has the nucleotide sequences of SEQ ID NOs:91-130 and SEQ ID NOs:132-160; SEQ ID NOs:162-170 and SEQ ID NOs:172-179; SEQ ID NOs:245-267 and SEQ ID NOs:269-287; or SEQ ID NOs:289-293 and SEQ ID NOs:295-307. In other aspects, the collection of second reverse primers has the sequences of CGACTCAAGTGTGTGGXXXXXXGGGTCAGGGTTCTGGATAT (SEQ ID NO:744) and CGACTCAGATTGGTACXXXXXXACACSTTKTTCAGGTCCTC (SEQ ID NO:745); or CGACTCAAGTGTGTGGXXXXXXTTCTGGGTTCTGGATGT (SEQ ID NO:746) and CGACTCAGATTGGTACXXXXXXAGTCACATTTCTCAGATCCT (SEQ ID NO:747), wherein XXXXXX is a unique barcode. In particular aspects, the collection of second reverse primers has the sequences of SEQ ID NOs:360-455 and SEQ ID NOs:456-551; or SEQ ID NOs:552-647 and SEQ ID NOs:648-743. In alternative aspects, the kit further includes comprising CITE-Seq primers, e.g., primers having the sequences of SEQ ID NO:356and SEQ ID NO:357; or SEQ ID NO:358 and SEQ ID NO:359.

The following non-limiting examples are provided to further illustrate the present invention.

### Example 1: Primers

The unbiased paired analysis of T-cell receptor (TCR) α- and β-chain usage at the single-cell level provides a valuable window for understanding the TCR repertoire and the nature of the immune response that would otherwise be difficult to obtain. Earlier technologies for TCR repertoire analysis were often limited to examining TCR complementarity-determining region 3 (CDR3) β expression or required in vitro cloning procedures that can artificially skew the TCR repertoire from the in vivo state. The protocol described here is a direct ex vivo, single-cell-based strategy for the clonotypic analysis of TCRαβ repertoires that uses multiplexed panels of CDR3α- and CDR3β-specific primers in a nested PCR to amplify transcripts from an individual, epitope-specific, or naïve T cell by an next generation sequencing method.

Human T Cell Receptors. External primers targeting human T cell receptor α (hTRA), β (hTRB), γ (hTRG), and λ (hTRD) genes for the first round of PCR amplification are provided in Tables 1, 2, 3, and 4, respectively.

TABLE 1

| hTRA gene (s) | External primer | SEQ |
|---|---|---|
| targeted by | sequence | ID |
| primer | (5′->3′) | NO: |
| Forward | | |
| hTRAV1-ext | AACTGCACGTACCAGACATC | 1 |
| hTRAV2-ext | GATGTGCACCAAGACTCC | 2 |
| hTRAV3-ext | AAGATCAGGTCAACGTTGC | 3 |
| hTRAV4-ext | CTCCATGGACTCATATGAAGG | 4 |
| hTRAV5-ext | CTTTTCCTGAGTGTCCGAG | 5 |
| hTRAV6-ext | CACCCTGACCTGCAACTATAC | 6 |
| hTRAV7-ext | GCAAAATACAGGGATGGG | 7 |
| hTRAV8-1-ext | CTCACTGGAGTTGGGATG | 8 |
| hTRAV8-3-ext | CACTGTCTCTGAAGGAGCC | 9 |
| hTRAV8-2,4-ext | GCCACCCTGGTTAAAGG | 10 |
| hTRAV8-6-ext | GAGCTGAGGTGCAACTACTC | 11 |
| hTRAV8-7-ext2 | CTAACAGAGGCCACCCAG | 12 |
| hTRAV9-1_2-ext | TGGTATGTCCAATATCCTGG | 13 |
| hTRAV10-ext | CAAGTGGAGCAGAGTCCTC | 14 |
| hTRAV12-1_3-ext | CARTGTTCCAGAGGGAGC | 15 |
| hTRAV13-1-ext | CATCCTTCAACCCTGAGTG | 16 |
| hTRAV13-2-ext | CAGCGCCTCAGACTACTTC | 17 |
| hTRAV14-ext | AAGATAACTCAAACCCAACCAG | 18 |
| hTRAV16-ext | AGTGGAGCTGAAGTGCAAC | 19 |
| hTRAV17-ext | GGAGAAGAGGATCCTCAGG | 20 |
| hTRAV18-ext3 | TCCAGTATCTAAACAAAGAGCC | 21 |
| hTRAV19-ext | AGGTAACTCAAGCGCAGAC | 22 |
| hTRAV20-ext | CACAGTCAGCGGTTTAAGAG | 23 |
| hTRAV21-ext | TTCCTGCAGCTCTGAGTG | 24 |
| hTRAV22-ext | GTCCTCCAGACCTGATTCTC | 25 |
| hTRAV23-ext | TGCTTATGAGAACACTGCG | 26 |
| hTRAV24-ext | CTCAGTCACTGCATGTTCAG | 27 |
| hTRAV25-ext | GGACTTCACCACGTACTGC | 28 |
| hTRAV26-1-ext | GCAAACCTGCCTTGTAATC | 29 |
| hTRAV26-2-ext | AGCCAAATTCAATGGAGAG | 30 |
| hTRAV27-ext | TCAGTTTCTAAGCATCCAAGAG | 31 |
| hTRAV29-ext | GCAAGTTAAGCAAAATTCACC | 32 |
| hTRAV30-ext | CAACAACCAGTGCAGAGTC | 33 |
| hTRAV34-ext | AGAACTGGAGCAGAGTCCTC | 34 |
| hTRAV35-ext | GGTCAACAGCTGAATCAGAG | 35 |
| hTRAV36-ext | GAAGACAAGGTGGTACAAAGC | 36 |
| hTRAV38-ext | GCACATATGACACCAGTGAG | 37 |
| hTRAV39-ext | CTGTTCCTGAGCATGCAG | 38 |
| hTRAV40-ext | GCATCTGTGACTATGAACTGC | 39 |
| hTRAV41-ext | AATGAAGTGGAGCAGAGTCC | 40 |
| Reverse | | |
| hTRAC-ext | GACCAGCTTGACATCACAG | 41 |

Primers targeting hTRAV are sense.
Primers targeting hTRAC genes are antisense.
hTRAV, human T cell receptor Vα; hTRAC, human T cell receptor Cα.

TABLE 2

| | | SEQ |
|---|---|---|
| hTRB gene (s) | External primer sequence | ID |
| targeted by primer | (5′->3′) | NO: |
| Forward | | |
| hTRBV2-ext | TCGATGATCAATTCTCAGTTG | 42 |
| hTRBV3-ext | CAAAATACCTGGTCACACAG | 43 |
| hTRBV4-ext | TCGCTTCTCACCTGAATG | 44 |
| hTRBV5-1_4-ext | GATTCTCAGGKCKCCAGTTC | 45 |
| hTRBV5-5_8-ext | GTACCAACAGGYCCTGGGT | 46 |
| hTRBV6-1_3,5_9-ext | ACTCAGACCCCAAAATTCC | 47 |
| hTRBV6-4-ext | ACTGGCAAAGGAGAAGTCC | 48 |
| hTRBV7-1_3-ext | TRTGATCCAATTTCAGGTCA | 49 |
| hTRBV7-4_9-ext new | CGSWTCTYTGCAGARAGGC | 50 |
| hTRBV9-ext | GATCACAGCAACTGGACAG | 51 |
| hTRBV10-1-ext | CAGAGCCCAAGACACAAG | 52 |
| hTRBV10-2-ext | ACCTTGATGTGTCACCAGAC | 53 |
| hTRBV10-3-ext | CAGAGCCCAAGACACAAG | 54 |
| hTRBV11-ext | CGATTTTCTGCAGAGACGC | 55 |
| hTRBV12-ext | ARGTGACAGARATGGGACAA | 56 |
| hTRBV13-ext | AGCGATAAAGGAAGCATCC | 57 |
| hTRBV14-ext | CCAACAATCGATTCTTAGCTG | 58 |
| hTRBV15-ext | AGTGACCCTGAGTTGTTCTC | 59 |
| hTRBV16-ext | GTCTTTGATGAAACAGGTATGC | 60 |
| hTRBV17-ext | CAGACCCCCAGACACAAG | 61 |
| hTRBV18-ext | CATAGATGAGTCAGGAATGCC | 62 |
| hTRBV19-ext | AGTTGTGAACAGAATTTGAACC | 63 |
| hTRBV20-ext | AAGTTTCTCATCAACCATGC | 64 |
| hTRBV23-ext | GCGATTCTCATCTCAATGC | 65 |
| hTRBV24-ext | CCTACGGTTGATCTATTACTCC | 66 |
| hTRBV25-ext | ACTACACCTCATCCACTATTCC | 67 |
| hTRBV27,28-ext | TGGTATCGACAAGACCCAG | 68 |
| hTRBV29-ext | TTCTGGTACCGTCAGCAAC | 69 |
| hTRBV30-ext | TCCAGCTGCTCTTCTACTCC | 70 |
| Reverse | | |
| hTRBC-ext | TAGAACTGGACTTGACAGCG | 71 |

Primers targeting hTRBV genes are sense.
Primers targeting hTRBC genes are antisense.
hTRBV, human T cell receptor Vβ; hTRBC, human T cell receptor Cβ.

TABLE 3

| | | SEQ |
|---|---|---|
| hTRG gene (s) | External primer sequence | ID |
| targeted by primer | (5′->3′) | NO: |
| Forward | | |
| hTRGV3.5-ext | TCTTCCAACTTGGAAGGG | 72 |
| hTRGV7-ext | TCTTCCAACTTGCAAGGG | 73 |
| hTRGVA-ext | GGGTCATCCTGTTTCCAG | 74 |
| hTRGVB-ext | TGGCCTCCCAAAGTACTG | 75 |
| hTRGV8-ext | CCAACTTGGAAGGGAGAAC | 76 |
| hTRGV9-ext | CCAGGTCACCTAGAGCAAC | 77 |
| hTRGV10-ext | TTATCAAAAGTGGAGCAGTTC | 78 |
| hTRGV11-ext | GAACAACCTGAAATATCTATTTCC | 79 |
| hTRGV1.2.4.6-ext | GGGTCATCTGCTGAAATCAC | 80 |
| Reverse | | |
| hTRGC-ext | GGTGTTCCCCTCCTGG | 81 |

Primers targeting hTRGV are sense.
Primers targeting hTRGC genes are antisense.
hTRGV, human T cell receptor Vγ; hTRGC, human T cell receptor Cγ.

TABLE 4

| hTRD gene (s) | | SEQ |
|---|---|---|
| targeted | External primer sequence | ID |
| by primer | (5′->3′) | NO: |
| Forward | | |
| hTRDV1-ext | GCCCAGAAGGTTACTCAAG | 82 |
| hTRDV2-ext | ATTGAGTTGGTGCCTGAAC | 83 |
| hTRDV3-ext | TGTGACAAAGTAACCCAGAGTTC | 84 |
| hTRDV4-ext | CAAACCCAACCAGGAATG | 85 |
| hTRDV5-ext | GCAAGTTAAGCAAAATTCACC | 86 |
| hTRDV6-ext | TTGATAGTCCAGAAAGGAGG | 87 |
| hTRDV7-ext | GACAAGGTGGTACAAAGCC | 88 |
| hTRDV8-ext | CAGTCACTCAGTCTCAACCAG | 89 |
| Reverse | | |
| hTRDC-ext | CTTCATATTTACCAAGCTTGACAG | 90 |

Primers targeting hTRDV are sense.
Primers targeting hTRDC genes are antisense.
hTRDV, human T cell receptor Vλ; hTRDC, human T cell receptor Cλ.

Internal primers for nested PCR amplification of hTRA, hTRB, hTRG, and hTRD amplicons in the second round of PCR amplification are provided in Tables 5, 6, 7, and 8, respectively.

TABLE 5

| | | SEQ |
|---|---|---|
| hTRA gene (s) | Internal primer sequence | ID |
| targeted by primer | (5′->3′) | NO: |
| Forward | | |
| hTRAV1-int | GCACCCACATTTCTKTCTTAC | 91 |
| hTRAV2-int | CACTCTGTGTCCAATGCTTAC | 92 |
| hTRAV3-int | ATGCACCTATTCAGTCTCTGG | 93 |
| hTRAV4-int | ATTATATCACGTGGTACCAACAG | 94 |
| hTRAV5-int | TACACAGACAGCTCCTCCAC | 95 |
| hTRAV6-int | TGGTACCGACAAGATCCAG | 96 |
| hTRAV7-int | TATGAGAAGCAGAAAGGAAGAC | 97 |
| hTRAV8-1-int | GTCAACACCTTCAGCTTCTC | 98 |
| hTRAV8-2, 8-4-int | TTTGAGGCTGAATTTAAGAGG | 99 |
| hTRAV8-3-int | AGAGTGAAACCTCCTTCCAC | 100 |
| hTRAV8-6-int | AACCAAGGACTCCAGCTTC | 101 |
| hTRAV8-7-int | ATCAGAGGTTTTGAGGCTG | 102 |
| hTRAV9-1, 9-2-int | GAAACCACTTCTTTCCACTTG | 103 |
| hTRAV10-int | GAAAGAACTGCACTCTTCAATG | 104 |
| hTRAV12-1, 12-2, | AAGATGGAAGGTTTACAGCAC | 105 |
| 12-3-int | | |
| hTRAV13-1-int | TCAGACAGTGCCTCAAACTAC | 106 |
| hTRAV13-2-int | CAGTGAAACATCTCTCTCTGC | 107 |
| hTRAV14-int | AGGCTGTGACTCTGGACTG | 108 |
| hTRAV16-int | GTCCAGTACTCCAGACAACG | 109 |
| hTRAV17-int | CCACCATGAACTGCAGTTAC | 110 |
| hTRAV18-int | TGACAGTTCCTTCCACCTG | 111 |
| hTRAV19-int | TGTGACCTTGGACTGTGTG | 112 |
| hTRAV20-int | TCTGGTATAGGCAAGATCCTG | 113 |
| hTRAV21-int | AACTTGGTTCTCAACTGCAG | 114 |
| hTRAV22-int | CTGACTCTGTGAACAATTTGC | 115 |
| hTRAV23-int | TGCATTATTGATAGCCATACG | 116 |
| hTRAV24-int | TGCCTTACACTGGTACAGATG | 117 |
| hTRAV25-int | TATAAGCAAAGGCCTGGTG | 118 |
| hTRAV26-1-int | CGACAGATTCACTCCCAG | 119 |
| hTRAV26-2-int | TTCACTTGCCTTGTAACCAC | 120 |
| hTRAV27-int | CTCACTGTGTACTGCAACTCC | 121 |
| hTRAV29-int | CTGCTGAAGGTCCTACATTC | 122 |
| hTRAV30-int | AGAAGCATGGTGAAGCAC | 123 |
| hTRAV34-int | ATCTCACCATAAACTGCACG | 124 |
| hTRAV35-int | ACCTGGCTATGGTACAAGC | 125 |
| hTRAV36-int | ATCTCTGGTTGTCCACGAG | 126 |
| hTRAV38-int | CAGCAGGCAGATGATTCTC | 127 |
| hTRAV39-int | TCAACCACTTCAGACAGACTG | 128 |
| hTRAV40-int | GGAGGCGGAAATATTAAAGAC | 129 |
| hTRAV41-int | TTGTTTATGCTGAGCTCAGG | 130 |
| Reverse | | |
| hTRAC-int | TGTTGCTCTTGAAGTCCATAG | 131 |

Primers targeting hTRAV are sense.
Primers targeting hTRAC are antisense.
hTRAV, human T cell receptor Vα; hTRAC, human T cell receptor Cα.

TABLE 6

| | | SEQ |
|---|---|---|
| hTRB gene (s) | Internal primer | ID |
| targeted by primer | sequence (5′->3′) | NO: |
| Forward | | |
| hTRBV2-int | TTCACTCTGAAGATCCGGTC | 132 |
| hTRBV3-int | AATCTTCACATCAATTCCCTG | 133 |
| hTRBV4-int | CCTGCAGCCAGAAGACTC | 134 |
| hTRBV5-1, 5-2, | CTTGGAGCTGGRSGACTC | 135 |
| 5-3, 5-4-int | | |
| hTRBV5-5, 5-6, | TCTGAGCTGAATGTGAACG | 136 |
| 5-7, 5-8-int | | |
| hTRBV6-1, 6-2, | GTGTRCCCAGGATATGAACC | 137 |
| 6-3, 6-5, 6-6, | | |
| 6-7, 6-8, 6-9-int | | |
| hTRBV6-4-int | TGGTTATAGTGTCTCCAGAGC | 138 |
| hTRBV7-1, 7-2, 7-3-int | TCYACTCTGAMGWTCCAGCG | 139 |
| hTRBV7-4, 7-5, 7-6, | TGRMGATYCAGCGCACA | 140 |
| 7-7, 7-8, 7-9-int | | |
| hTRBV9-int | GTACCAACAGAGCCTGGAC | 141 |
| hTRBV10-1-int | TGGTATCGACAAGACCTGG | 142 |
| hTRBV10-2-int | TGGTATCGACAAGACCTGG | 143 |
| hTRBV10-3-int | GGAACACCAGTGACTCTGAG | 144 |
| hTRBV11-int | GACTCCACTCTCAAGATCCA | 145 |
| hTRBV12-int | CYACTCTGARGATCCAGCC | 146 |
| hTRBV13-int | CATTCTGAACTGAACATGAGC | 147 |
| hTRBV14-int | ATTCTACTCTGAAGGTGCAGC | 148 |
| hTRBV15-int | ATAACTTCCAATCCAGGAGG | 149 |
| hTRBV16-int | CTGTAGCCTTGAGATCCAGG | 150 |
| hTRBV17-int | TGTTCACTGGTACCGACAG | 151 |
| hTRBV18-int | CGATTTTCTGCTGAATTTCC | 152 |
| hTRBV19-int | TTCCTCTCACTGTGACATCG | 153 |
| hTRBV20-int | ACTCTGACAGTGACCAGTGC | 154 |
| hTRBV23-int | GCAATCCTGTCCTCAGAAC | 155 |
| hTRBV24-int | GATGGATACAGTGTCTCTCGA | 156 |
| hTRBV25-int | CAGAGAAGGGAGATCTTTCC | 157 |
| hTRBV27, 28-int | TTCYCCCTGATYCTGGAGTC | 158 |
| hTRBV29-int | TCTGACTGTGAGCAACATGAG | 159 |
| hTRBV30-int | AGAATCTCTCAGCCTCCAGAC | 160 |
| Reverse | | |
| hTRBC-int | TTCTGATGGCTCAAACACAG | 161 |

Primers targeting hTRBV genes are sense.
Primers targeting hTRBC genes are antisense.
hTRBV, human T cell receptor Vβ; hTRBC, human T cell receptor Cβ.

TABLE 7

| hTRG gene (s) | Internal primer | SEQ ID |
|---|---|---|
| targeted by primer | sequence (5′->3′) | NO: |
| Forward | | |
| hTRGV3.5-int | GGTCATCTGCTGAAATCAC | 162 |
| hTRGV7-int | GGTCATCTGCTGTAATCACTTG | 163 |
| hTRGVA-int | TACCTAAGGACCTGTGTAGAGG | 164 |
| hTRGVB-int | TCCTCTTTCTATGTCCCAGG | 165 |
| hTRGV8-int | AAAATGCCGTCTACACCC | 166 |
| hTRGV9-int | TGTCCATTTCATATGACGG | 167 |
| hTRGV10-int | CAGCTATCCATTTCCACGG | 168 |
| hTRGV11-int | CATATCTTGGAAGGCATCC | 169 |
| hTRGV1.2.4.6-int | CCAGGAGGGGAAGGC | 170 |
| Reverse | | |
| hTRGC-int | CCCAGAATCGTGTTGCT | 171 |

Primers targeting hTRGV are sense.
Primers targeting hIRGC genes are antisense.
hTRGV, human T cell receptor Vγ; hTRGC, human T cell receptor Cγ.

TABLE 8

| hTRD gene (s) | | SEQ |
|---|---|---|
| targeted | Internal primer sequence | ID |
| by primer | (5′->3′) | NO: |
| Forward | | |
| hTRDV1-int | AGCAAAGAGATGATTTTCCTTA | 172 |
| hTRDV2-int | TATATCAACTGGTACAGGAAGACC | 173 |
| hTRDV3-int | GGTACTGCTCTGCACTTACGAC | 174 |
| hTRDV4-int | AGGAAAAGGAGGCTGTGAC | 175 |
| hTRDV5-int | CTGCTGAAGGTCCTACATTC | 176 |
| hTRDV6-int | CGTTTGACTACTTTCCATGG | 177 |
| hTRDV7-int | ATCTCTGGTTGTCCACGAG | 178 |
| hTRDV8-int | TCTGGTACAAGCAGCCTC | 179 |
| Reverse | | |
| hTRDC-int | GATGACAATAGCAGGATCAAAC | 180 |

Primers targeting hTRDV are sense.
Primers targeting hTRDC genes are antisense.
hTRDV, human T cell receptor Vλ; hTRDC, human T cell receptor Cλ.

Mouse T Cell Receptors. External primers targeting mouse T cell receptor α (mTRA), β (mTRB), γ (mTRG), and λ (mTRD) genes for the first round of PCR amplification are provided in Tables 9, 10, 11, and 12, respectively.

TABLE 9

| mTRA gene (s) | | SEQ |
|---|---|---|
| targeted | External primer sequence | ID |
| by primer | (5′->3′) | NO: |
| Forward | | |
| mTRAV1-ext | GGTTATCCTGGTACCAGCA | 181 |
| mTRAV2-ext | CATCTACTGGTACCGACAGG | 182 |
| mTRAV3-ext | GGCGAGCAGGTGGAG | 183 |
| mTRAV4-ext | TCTGSTCTGAGATGCAATTTT | 184 |
| mTRAV5-1/5-4(D)-ext | GGCTACTTCCCTTGGTATAAGCAAGA | 185 |
| mTRAV6-1/6-2-ext | CAGATGCAAGGTCAAGTGAC | 186 |
| mTRAV6-3/6-4(D)-ext | AAGGTCCACAGCTCCTTC | 187 |
| mTRAV6-5/6-7(D)-ext | GTTCTGGTATGTGCAGTATCC | 188 |
| mTRAV6-6-ext | AGATTCCGTGACTCAAACAG | 189 |
| mTRAV7-ext | AGAAGGTRCAGCAGAGCCCAGAATC | 190 |
| mTRAV8-ext | GAGCRTCCASGAGGGTG | 191 |
| mTRAV9-ext | CCAGTGGTTCAAGGAGTG | 192 |
| mTRAV10/10a(D)-ext | AGAGAAGGTCGAGCAACAC | 193 |
| mTRAV11-ext | AAGACCCAAGTGGAGCAG | 194 |
| mTRAV12-ext | TGACCCAGACAGAAGGC | 195 |
| mTRAV13-ext | TCCTTGGTTCTGCAGG | 196 |
| mTRAV14-ext | GCAGCAGGTGAGACAAAG | 197 |
| mTRAV15-ext | CASCTTYTTAGTGGAGAGATGG | 198 |
| mTRAV16-ext | GTACAAGCAAACAGCAAGTG | 199 |
| mTRAV17-ext | CAGTCCGTGGACCAGC | 200 |
| mTRAV18-ext | AACGGCTGGAGCAGAG | 201 |
| mTRAV19-ext | GCAAGTTAAACAAAGCTCTCC | 202 |
| mTRAV21-ext | GTGCACTTGCCTTGTAGC | 203 |
| Reverse | | |
| mTRAC-ext | GGCATCACAGGGAACG | 204 |

Primers targeting mTRAV are sense.
Primers targeting mTRAC genes are antisense.
mTRAV, mouse T cell receptor Vα; mTRAC, mouse T cell receptor Cα.

TABLE 10

| mTRB gene (s) | | |
|---|---|---|
| targeted by | External primer | SEQ ID |
| primer | sequence (5′->3′) | NO: |
| Forward | | |
| mTRBV1-ext | TACCACGTGGTCAAGCTG | 205 |
| mTRBV2-ext | CAGTATCTAGGCCACAATGC | 206 |
| mTRBV3-ext | CCCAAAGTCTTACAGATCCC | 207 |
| mTRBV4-ext | GACGGCTGTTTTCCAGAC | 208 |
| mTRBV5-ext | GGTATAAACAGAGCGCTGAG | 209 |
| mTRBV12-ext | GGGGTTGTCCAGTCTCC | 210 |
| mTRBV13-ext | GCTGCAGTCACCCAAAG | 211 |
| mTRBV14-ext | GCAGTCCTACAGGAAGGG | 212 |
| mTRBV15-ext | GAGTTACCCAGACACCCAG | 213 |
| mTRBV16-ext | CCTAGGCACAAGGTGACAG | 214 |
| mTRBV17-ext | GAAGCCAAACCAAGCAC | 215 |
| mTRBV19-ext | GATTGGTCAGGAAGGGC | 216 |
| mTRBV20-ext | GGATGGAGTGTCAAGCTG | 217 |
| mTRBV23-ext | CTGCAGTTACACAGAAGCC | 218 |
| mTRBV24-ext | CAGACTCCACGATACCTGG | 219 |
| mTRBV26-ext | GGTGAAAGGGCAAGGAC | 220 |
| mTRBV29-ext | GCTGGAATGTGGACAGG | 221 |
| mTRBV30-ext | CCTCCTCTACCAAAAGCC | 222 |
| mTRBV31-ext | CTAACCTCTACTGGTACTGGCAG | 223 |
| Reverse | | |
| mTRBC-ext | CCAGAAGGTAGCAGAGACCC | 224 |

Primers targeting mTRBV genes are sense.
Primers targeting mTRBC genes are antisense.
mTRBV, mouse T cell receptor Vβ; mTRBC, mouse T cell receptor Cβ.

TABLE 11

| mTRG gene (s) | | SEQ |
|---|---|---|
| targeted | External primer sequence | ID |
| by primer | (5′->3′) | NO: |
| Forward | | |
| mTRGV1-3-ext | GCAGCTGGAGCAAACTG | 225 |
| mTRGV4-ext | CAAATATCCTGTAAAGTTTTCATC | 226 |
| mTRGV5-ext | GATATCTCAGGATCAGCTCTCC | 227 |
| mTRGV6-ext | TCACCTCTGGGGTCATATG | 228 |
| mTRGV7-ext | CAACTTGGAAGAAAGAATAATGTC | 229 |
| Reverse | | |
| mTRGC-ext | CTTTTCTTTCCAATACACCC | 230 |

Primers targeting mTRGV are sense.
Primers targeting mTRGC genes are antisense.
mTRGV, mouse T cell receptor Vγ; mIRGC, mouse T cell receptor Cγ.

TABLE 12

| mTRD gene (s) | | SEQ |
|---|---|---|
| targeted | External primer sequence | ID |
| by primer | (5′->3′) | NO: |
| Forward | | |
| mTRDV1-ext | ACCCAAATGTTGCATCAG | 231 |
| mTRDV2-ext | TCTGTGCAGGTGGCAG | 232 |
| mTRDV4-ext | TGTATATTTGGAACCAGTTGC | 233 |
| mTRDV5-ext | CATCACGCTGACCCAG | 234 |
| mTRDV6.AV15-ext | CASCTTYTTAGTGGAGAGATGG | 235 |
| mTRDV7.AV13-ext | TCCTTGGTTCTGCAGG | 236 |
| mTRDV8.AV14-ext | GCAGCAGGTGAGACAAAG | 237 |
| mTRDV9.AV6-1.6-2-ext | CAGATGCAAGGTCAAGTGAC | 238 |
| mTRDV9.AV6-3.6-4-ext | AAGGTCCACAGCTCCTTC | 239 |
| mTRDV9.AV6-5.6-7-ext | GTTCTGGTATGTGCAGTATCC | 240 |
| mTRDV10.AV4-ext | TCTGSTCTGAGATGCAATTTT | 241 |
| mTRDV11.AV16-ext | GTACAAGCAAACAGCAAGTG | 242 |
| mTRDV12/TRAV21-ext | GTGCACTTGCCTTGTAGC | 243 |
| Reverse | | |
| mTRDC-ext | TGAAAGAATTTTGCATATGGTTC | 244 |

Primers targeting mTRDV are sense.
Primers targeting mTRDC genes are antisense.
mTRDV, mouse T cell receptor Vλ; mTRDC, mouse T cell receptor Cλ.

Internal primers for nested PCR amplification of mTRA, mTPRB, mTRG, and mTRD amplicons in the second round of PCR amplification are provided in Tables 13, 14, 15, and 16, respectively.

TABLE 13

| mTRA gene (s) | | SEQ |
|---|---|---|
| targeted | Internal primer sequence | ID |
| by primer | (5′->3′) | NO: |
| Forward | | |
| mTRAV1-int | CTCCACATTCCTGAGCC | 245 |
| mTRAV2-int | ACTCTGAGCCTGCCCT | 246 |
| mTRAV3-int | GCCCTCCTCACCTGAG | 247 |
| mTRAV4-int | GGITIMAGGAACAAAGGAGAAT | 248 |
| mTRAV5-1/5-4(D)-int | ATYCGTTCAAATATGGAAAGAAA | 249 |
| mTRAV6-1/6-2-int | GGAGAAGGTCCACAGCTC | 250 |
| mTRAV6-3/6-4(D)-int | CAACTGCCAACAACAAGG | 251 |
| mTRAV6-5/6-7(D)-int | TCCTTCCACTTGCAGAAAG | 252 |
| mTRAV6-6-int | ACGGCTGGCCAGAAG | 253 |
| mTRAV7-int | CAKGRCYTCYYTCAACTGCAC | 254 |
| mTRAV8-int | AGAGCCACCCTTGACAC | 255 |
| mTRAV9-int | GCTTYGAGGCTGAGTTCAG | 256 |
| mTRAV10/10a(D)-int | CTACACTGAGTGTTCGAGAGG | 257 |
| mTRAV11-int | AACAGGACACAGGCAAAG | 258 |
| mTRAV12-int | GGTTCCACGCCACTC | 259 |
| mTRAV13-int | TGCAGGAGGGGGAGA | 260 |
| mTRAV14-int | CTCTGACAGTCTGGGAAGG | 261 |
| mTRAV15-int | AYTCTGTAGTCTTCCAGAAATCAC | 262 |
| mTRAV16-int | ATTATTCTCTGAACTTTCAGAAGC | 263 |
| mTRAV17-int | TATGAAGGAGCCTCCCTG | 264 |
| mTRAV18-int | CAAGATTTCACCGCACG | 265 |
| mTRAV19-int | GCTGACTGTTCAAGAGGGA | 266 |
| mTRAV21-int | AATAGTATGGCTTTCCTGGC | 267 |
| Reverse | | |
| mTRAC-int | GCACATTGATTTGGGAGTC | 268 |

Primers targeting mTRAV are sense.
Primers targeting mTRAC genes are antisense.
mTRAV, mouse T cell receptor Vα; mTRAC, mouse T cell receptor Cα.

TABLE 14

| mTRB gene (s) | Internal primer | |
|---|---|---|
| targeted by | sequence | SEQ ID |
| primer | (5′->3′) | NO: |
| Forward | | |
| mTRBV1-int | GTATCCCTGGATGAGCTG | 269 |
| mTRBV2-int | GGACAATCAGACTGCCTC | 270 |
| mTRBV3-int | GATATGGGGCAGATGGTG | 271 |
| mTRBV4-int | CAGGTGGGAAATGAAGTG | 272 |
| mTRBV5-int | GCCAGAGCTCATGTTTCTC | 273 |
| mTRBV12-int | CCAGCAGATTCTCAGTCC | 274 |
| mTRBV13-int | GTACTGGTATCGGCAGGAC | 275 |
| mTRBV14-int | GGTATCAGCAGCCCAGAG | 276 |
| mTRBV15-int | GTGTGAGCCAGTTTCAGG | 277 |
| mTRBV16-int | GAAGCAACTCTGTGGTGTG | 278 |
| mTRBV17-int | GAACAGGGAAGCTGACAC | 279 |
| mTRBV19-int | GGTACCGACAGGATTCAG | 280 |
| mTRBV20-int | GCTTGGTATCGTCAATCG | 281 |
| mTRBV23-int | GCCAGGAAGCAGAGATG | 282 |
| mTRBV24-int | GCACACTGCCTTTTACTGG | 283 |
| mTRBV26-int | GAGGTGTATCCCTGAAAAGG | 284 |
| mTRBV29-int | GTACTGGTATCGACAAGACCC | 285 |
| mTRBV30-int | GGACATCTGTCAAAGTGGC | 286 |
| mTRBV31-int | CTGTTGGCCAGGTAGAGTC | 287 |
| Reverse | | |
| mTRBC-int | GGGTAGCCTTTTGTTTGTTTG | 288 |

Primers targeting mTRBV genes are sense.
Primers targeting mTRBC genes are antisense.
mTRBV, mouse T cell receptor Vβ; mTRBC, mouse T cell receptor Cβ.

TABLE 15

| mTRG gene (s) | | SEQ |
|---|---|---|
| targeted by | Internal primer sequence | ID |
| primer | (5′->3′) | NO: |
| Forward | | |
| mTRGV1-3-int | CTGAATTATCGGTCACCAG | 289 |
| mTRGV4-int | GTTTAGAGTTTCTATTATATGTCCTTGCAAC | 290 |
| mTRGV5-int | TACCCGAAGACCAAACAAGAC | 291 |
| mTRGV6-int | AGAGGAAAGGAAATACGGC | 292 |
| mTRGV7-int | CACCAAGCTAGAGGGGTC | 293 |
| Reverse | | |
| mTRGC-int | TCDGGAAAGAACTTTTCAAGG | 294 |

Primers targeting mTRGV are sense.
Primers targeting mTRGC genes are antisense.
mTRGV, mouse T cell receptor Vγ; mTRGC, mouse T cell receptor Cγ.

TABLE 16

| | | SEQ |
|---|---|---|
| mTRD gene (s) | Internal primer sequence | ID |
| targeted by primer | (5′->3′) | NO: |
| Forward | | |
| mTRDV1-int | GTCTCTGACAATCCAAGAAGG | 295 |
| mTRDV2-int | CCCTGGACTGCACCTATG | 296 |
| mTRDV4-int | GATCCTGCCTCCTTCTACTG | 297 |
| mTRDV5-int | GCTCCACTGACCAGACAG | 298 |
| mTRDV6.AV15-int | AYTCTGTAGTCTTCCAGAAATCAC | 299 |
| mTRDV7.AV13-int | TGCAGGAGGGGGAGA | 300 |
| mTRDV8.AV14-int | CTCTGACAGTCTGGGAAGG | 301 |
| mTRDV9.AV6-1.6-2-int | GGAGAAGGTCCACAGCTC | 302 |
| mTRDV9.AV6-3.6-4-int | CAACTGCCAACAACAAGG | 303 |
| mTRDV9.AV6-5.6-7-int | TCCTTCCACTTGCAGAAAG | 304 |
| mTRDV10.AV4-int | GGITIMAGGAACAAAGGAGAAT | 305 |
| mTRDV11.AV16-int | ATTATTCTCTGAACTTTCAGAAGC | 306 |
| mTRDV12.AV21-int | AATAGTATGGCTTTCCTGGC | 307 |
| Reverse | | |
| mTRDC-int | GAGATGACTATAGCAGGGTCG | 308 |

Primers targeting mTRDV are sense.
Primers targeting mTRDC genes are antisense.
mTRDV, mouse T cell receptor Vλ; mTRDC, mouse T cell receptor Cλ.

Macaque T Cell Receptors. External primers targeting macaque T cell receptor α (macTRA) genes for the first round of PCR amplification are provided in Tables 17. External primers targeting macaque T cell receptor β (macTRB) genes for the first round of PCR amplification are the same as those used for hTRB (Table 2).

TABLE 17

| macTRA gene (s) | External primer | SEQ ID |
|---|---|---|
| targeted by primer | sequence (5′->3′) | NO: |
| Forward | | |
| macTRAV1-ext | ATMAACTGCACGTACCAGAC | 309 |
| hTRAV2-ext | GATGTGCACCAAGACTCC | 2 |
| hTRAV4-ext | CTCCATGGACTCATATGAAGG | 4 |
| hTRAV5-ext | CTTTTCCTGAGTGTCCGAG | 5 |
| hTRAV6-ext | CACCCTGACCTGCAACTACAC | 310 |
| macTRAV7-ext | CTATCAGGGGCCACCCAG | 311 |
| macTRAV8-1-ext | GTGCAACTATTCCTATAGTG | 312 |
| hTRAV8-3-ext | GTYKCTGTCTCTGAAGGAG | 313 |
| macTRAV8-2,4-ext | CCACCCWGGTTAAAGGC | 314 |
| hTRAV8-6-ext | CTGCTGAGGTGCAACTACTC | 315 |
| macTRAV8-7-ext2 | GTCTCCAGCTTCTCCTG | 316 |
| hTRAV9-1_2-ext | TGGTATGTCCAATATCCTGG | 13 |
| hTRAV10-ext | CAAGTGGAGCAGAGTCCTC | 14 |
| hTRAV12-1_3-ext | CARTGTTCCAGAGGGAGC | 15 |
| hTRAV13-1-ext | CATCCTTCAACCCTGAGTG | 16 |
| macTRAV13-2-ext | CAGTGCCTCARACTACTTC | 317 |
| hTRAV14-ext | AAGATAACTCAAACCCAACCAG | 18 |
| macTRAV16-ext | AGTGGAACTGAAGTGCTAC | 318 |
| hTRAV17-ext | GGAGAAGAGGATCCTCAGG | 20 |
| macTRAV18-ext3 | AATACCCCAACCAAGGTCTC | 319 |
| macTRAV19-ext | AGGTAACTCAAGCTCAGAC | 320 |
| macTRAV20-ext | CACAGTCAGCGGCTTAAGAG | 321 |
| hTRAV21-ext | TTCCTGCAGCTCTGAGTG | 24 |
| hTRAV22-ext | GTCCTCCAGACCTGATTCTC | 25 |
| hTRAV23-ext | GTGCTTATGAGAACAGTGC | 322 |
| macTRAV24-ext | CCAGTCACTGCATGTTCAG | 323 |
| hTRAV26-1-ext | GCAAACCTGCCTTGTAATC | 29 |
| hTRAV26-2-ext | AGCCAAATTCAATGGAGAG | 30 |
| macTRAV27-ext | CGGTTTCTAAGCATCCAAGAG | 324 |
| macTRAV29-ext | GCGAGTTAAGCAAAATCCAC | 325 |
| hTRAV30-ext | CAACAACCAGTGCAGAGTC | 33 |
| hTRAV34-ext | AGAACTGGAGCAGAGTCCTC | 34 |
| macTRAV36-ext | GAGCAGTGAAGACAAGGTG | 326 |
| macTRAV38-ext | TGCACATATGACACCAGTG | 327 |
| macTRAV39-ext | CTGTTCCTGAGCACGCAG | 328 |
| macTRAV41-ext | AAGTGGAGCAGAGTCCTC | 329 |
| Reverse | | |
| macTRAC-ext | ACCTCATGTCTAGCACAG | 330 |

Primers targeting macTRAV are sense.
Primers targeting macTRAC genes are antisense.
macTRAV, macaque T cell receptor Vα; macTRAC, macaque T cell receptor Cα.

Internal primers for nested PCR amplification of macTRA amplicons in the second round of PCR amplification are provided in Table 18. Internal primers targeting macTRB genes for the first round of PCR amplification are the same as those used for hTRB (Table 6).

TABLE 18

| | | SEQ |
|---|---|---|
| macTRA gene (s) | Internal primer | ID |
| targeted by primer | sequence (5′->3′) | NO: |
| Forward | | |
| macTRAV1-int | GCACCCACAYTTCTKTCTTAC | 331 |
| hTRAV2-int | CACTCTGTGTCCAATGCTTAC | 92 |
| macTRAV4-int | ATTATATCATGTGGTACCAACAG | 332 |
| macTRAV5-int | GGTATAAGCAAGAACCTGG | 333 |
| hTRAV6-int | TGGTACCGACAAGATCCAG | 96 |
| macTRAV7-int | GAGGCTGAATTTAAGAAGAG | 334 |
| macTRAV8-1-int | GCCAAAGCCTTGAGCTTCTC | 335 |
| macTRAV8-3-int | GYTTTGAGGCTGAATTTAAGA | 336 |
| macTRAV8-2,4-int | AGAGTGAAACYTCCTTCCAC | 337 |
| macTRAV8-6-int | AACCAAGGDCTCCRGCTTC | 338 |
| macTRAV8-7-int2 | ATCAAWGGTTTTGAGGCTG | 339 |
| hTRAV9-1_2-int | GAAACCACTTCTTTCCACTTG | 103 |
| hTRAV10-int | GAAAGAACTGCACTCTTCAATG | 104 |
| macTRAV12-1_3-int | AAGATGGAAGRTTTACAGCAC | 340 |
| macTRAV13-1-int | CTTATTCAGACAGTGCCTCA | 341 |
| macTRAV13-2-int | CAATGAAACATCTCTCTCTGC | 342 |
| hTRAV14-int | AGGCTGTGACTCTGGACTG | 108 |
| macTRAV16-int | GTCCAGTACCCCAAACAACG | 343 |
| hTRAV17-int | CCACCATGAACTGCAGTTAC | 110 |
| macTRAV18-int | TGAAACCTCCTTCCACCTG | 344 |
| hTRAV19-int | TGTGACCTTGGACTGTGTG | 112 |
| hTRAV20-int | TCTGGTATAGGCAAGATCCTG | 113 |
| hTRAV21-int | AACTTGGTTCTCAACTGCAG | 114 |
| macTRAV22-int | CTGCCACTGTGAACAATTTGC | 345 |
| macTRAV23-int | TGCATTATTGATAGCCATAAG | 346 |
| macTRAV24-int | TGCCTTGCACTGGTACAGATG | 347 |
| macTRAV26-1-int | CGACAGATTCACTCCGAG | 348 |
| macTRAV26-2-int | TACACTTGCCTTGTAACCAC | 349 |
| macTRAV27-int | TCACTGCGTACTGCAACTCC | 350 |
| hTRAV29-int | CTGCTGAAGGTCCTACATTC | 122 |
| macTRAV30-int | AGAAGCATGGTGAAGCG | 351 |
| macTRAV34-int | ATCTCACCATAAACTGCAC | 352 |
| hTRAV36-int | ATCTCTGGTTGTCCACGAG | 126 |
| hTRAV38-int | CAGCAGGCAGATGATTCTC | 127 |
| macTRAV39-int | TCAACCGCTTCAGACAGACTG | 353 |
| macTRAV41-int | GTGCAATTATTCTGCCACTG | 354 |
| Reverse | | |
| macTRAC-int | ATCCTTGCTTTGTGACAC | 355 |

Primers targeting macTRAV are sense.
Primers targeting macTRAC genes are antisense.
macTRAV, macaque T cell receptor Vα; macTRAC, macaque T cell receptor Cα.

Stocks of primers were prepared by resuspending primers to 200 μM using 1X TE buffer (low EDTA; 10 mM Tris-HCl, 0.1 mM EDTA, pH 8.0) and stored at −20° C.

Cocktails of external forward TRAV (TRAV EXT FOR) and internal forward TRAV (TRAV INT FOR) primers were prepared by combining 25 μl of each TRAV primer (200 μM stock), thereby yielding 1000 μl of diluted primer cocktail with each primer at 5 pmol/μl working concentration.

All reverse primers including TRAC external reverse (TRAC EXT REV), TRBC external reverse (TRBC EXT REV), TRAC internal reverse (TRAC INT REV), TRBC internal reverse (TRBC INT REV) were resuspended to prepare 20 μM working stocks.

### Example 2: Methods

Single Cell Sorting. CD8⁺ T cells are isolated from peripheral blood mononuclear cells (PBMC), Bronchoalveolar lavage (BAL), spleen (SPL), and lymph nodes (LN) of infected, naïve or memory animals (i.e., humans, mice, or macaque). Cells are resuspended in 1 ml of Dulbecco's phosphate-buffered saline (D-PBS) without Ca⁺⁺/Mg⁺⁺ or extraneous proteins. The cells are stained with LIVE/DEAD® Fixable Aqua Dead Cell Stain (405 nm excitation) discrimination dye for 30 minutes at room temperature in the dark. At the end of the incubation, cells are washed twice with sort buffer (PBS containing 0.1% BSA, fraction V (Life Technologies)) by centrifugation at 500 g and +4° C. for 5 minutes.

Cells are subsequently resuspended in 50 μl of sort buffer containing blocking antibody (anti-mouse CD16/CD32) or APC-conjugated peptide-loaded pMHCI tetramer (e.g., HLA-A*0201-CMV pp65; Beckman Coulter) in appropriate dilution and incubated at room temperature in the dark for 1 hour. The cells are again washed twice by centrifuging at 500 g and +4° C. for 5 minutes using sort buffer. For human cells, the cell pellet is resuspended in sort buffer containing an appropriate dilution of FITC-conjugated anti-human CD3, PE-Cy7-conjugated anti-human CD8, PE-conjugated anti-human CD14. For mouse cells, the cell pellet is resuspended in sort buffer containing an appropriate dilution of mouse-CD4, anti-mouse CD11b, anti-mouse CD11c, F4/80 (all Pacific Blue-conjugated for negative gating) (Biolegend), and anti-mouse CD8-APC-eFluro780 (eBiosciences). Subsequently, the cells are incubated on ice for 20 minutes in the dark.

Cells are again washed twice by centrifuging at 500 g and +4° C. for 5 minutes using sort buffer. The resulting pellets are resuspended in 0.5 ml of sort buffer containing RNase inhibitor at 200 U/ml. The cell suspension is filtered through a 40 μM cell strainer. Lymphocytes are first gated on their scatter properties based on a FSC-A/SSC-A plot. From the probable lymphocyte population, the live cells are selected based on Fixable LIVE/DEAD® aqua staining and CD3, CD8, and CD14 negative cells for human cells, or CD4, CD11b, CD11c and F4/80 negative cells for mouse cells. Cells are than gated on CD8⁺ tetramer⁺ for sorting.

Epitope-specific CD8 cells are sorted into each well of columns 1-23 of a 384-well polypropylene PCR plate. Column 24 of the 384-well plate is left empty for the negative (no template) control. Following the sort, the plate is sealed with adhesive plate seal (MicroAmp, Applied Biosystem) and placed on ice. The plates are briefly spun to bring the contents down and frozen at −80° C.

Reverse Transcription (RT). The reverse transcription of the TCRα and β mRNA is carried out directly on the lysed cells without any RNA extraction step. Lysis is achieved by a combination of the freeze-thaw cycle and inclusion of a detergent (TRITON™ X-100, 0.1% final) in the RT mixture. SUPERSCRIPT® VILO™ (ThermoFisher) is used for RT as follows. More specifically, the plate is thawed, centrifuged at 500 g for 2 minutes and kept on ice. An RT master mix is prepared as appropriate for the SUPERSCRIPT® VILO™ (Table 19).

TABLE 19

| | Per well | One Plate |
|---|---|---|
| Component | (Total 1 μl) | (450 reactions) |
| 5X RT Buffer | 0.2 μl | 90 μl |
| 10X SUPERSCRIPT ® RT enzyme | 0.1 μl | 45 μl |
| Water, Nuclease free | 0.6 μl | 270 μl |
| Triton-X100 (1%) | 0.1 μl | 45 μl |

To each well is added 2.5 μl of RT master mix using a multichannel pipette. Once all columns are filled, the plate is resealed and centrifuged at 500 g for 2 minutes. Using a thermocycler, cDNA is synthesized as follows: 5 minutes at 25° C.; 45 minutes at 42° C.; 5 minutes at 85° C.; hold at 4° C. After cDNA synthesis, the plate is stored at −20° C.

First Round of Polymerase chain reaction (PCR). Nested PCR is carried out to amplify the TCR chains from the single cells. The reaction mixture for the first round PCR (384-well plate) includes the primers listed in Tables 1-4 for human TCR chains, Tables 9-12 for mouse TCR chains, or Tables 2 and 17 for macaque TCR chains and the components listed in Table 20. Cellular Indexing of the Transcriptome and Epitopes by Sequencing (Cite-Seq) primers (Table 21) may also be included to simultaneously measure proteins and RNA at a single-cell level (Stoeckius et al. (2017) Nat Methods 14:865-868).

TABLE 20

| | | One Plate |
|---|---|---|
| Component | Per well | (450 reactions) |
| Water, Nuclease free | 6.9 μl | 3105 μl |
| PCR buffer, 10X | 1 μl | 450 μl |
| (Containing 15 mM MgCl₂) | | |
| dNTP, 10 mM | 0.2 μl | 90 μl |
| TRAC EXT REV (20 μM) | 0.2 μl | 90 μl |
| TRAV-EXT FOR | 0.2 μl | 90 μl |
| (Cocktail α primers) | | |
| TRBC EXT REV (20 μM) | 0.2 μl | 90 μl |
| TRBV EXT FOR | 0.2 μl | 90 μl |
| (Cocktail β primers) | | |
| MP_citeseq FOR | 0.2 μl | 90 μl |
| MP_citeseq REV | 0.2 μl | 90 μl |
| Taq DNA Polymerase | 0.1 μl | 45 μl |
| Total | 9 μl | 4050 μl |

TABLE 21

| MP_citeseq | Primer sequence | SEQ |
|---|---|---|
| primer | (5′ −> 3′) | ID NO: |
| Hum_FOR | ACACCTTGTTCAGGTCCTCCGGAGATGTGTATAAGAGACAG | 356 |
| Hum_REV | ACACGTTGTTCAGGTCCTCTTTCTTATATGGG | 357 |
| Mus_FOR | AGTCACATTTCTCAGATCCTCGGAGATGTGTATAAGAGACAG | 358 |
| Mus_REV | AGTCACATTTCTCAGATCCTTTTCTTATATGGG | 359 |

To each sample and control well of the cDNA plate is added 9 μl of the master mix. The plate is resealed and centrifuged to bring the contents to the bottom of the wells. PCR of human TCRαβ is carried out in a thermocycler as follows: initial denaturation at 95° C. for 5 minutes; 34 cycles of denaturation at 95° C. for 20 seconds, primer annealing at 52° C. for 20 seconds, polymerase extension at 72° for 45 seconds; a final extension at 72° C. for 7 minutes and a final hold at 4° C. PCR of mouse TCRαβ is carried out as described for human TCRαβ except that primer annealing is carried out at 55° C. instead of 52° C. The plates are stored at −20° C. until the next step.

Nested PCR. Before the second round of PCR, the 384 samples from the first round of PCR are split into four 96-well plates, wherein each well of each 96-well plate is uniquely barcoded. The reaction mixture for the second round PCR (four, 96-well plates) includes the primers listed in Tables 5-8 for human TCR chains, Tables 13-16 for mouse TCR chains, or Tables 6 and 18 for macaque TCR chains and the components listed in Table 22. To introduce C-segment-specific primers containing well-specific barcodes, pools of human or mouse TCRα and β C-segment-specific reverse primers containing well-specific barcodes are also included (Tables 23-26).

TABLE 22

| | | One Plate |
|---|---|---|
| Component | Per well | (450 reactions) |
| Water, Nuclease free | 6.9 μl | 3105 μl |
| PCR buffer, 10X | 1 μl | 450 μl |
| (Containing 15 mM MgCl₂) | | |
| dNTP, 10 mM | 0.2 μl | 90 μl |
| TRAV-INT + TRBV-INT | 0.2 μl | 90 μl |
| (cocktail of α and β | | |
| forward primers) | | |
| Acj_ind# (10 μM) | 0.2 μl | Unique index |
| | | per well |
| Bcj_ind# (10 μM) | 0.2 μl | Unique index |
| | | per well |
| Taq DNA Polymerase | 0.1 μl | 45 μl |
| Total | 9 μl | 4050 μl |

TABLE 23

| Plate | Ind | MP_Hum_Acj (Human TCRα reverse) Primer | SEQ ID |
|---|---|---|---|
| Well | # | sequence* (5′->3′) | NO: |
| A1 | 1 | CGACTCAAGTGTGTGGAACAGTGGGTCAGGGTTCTGGATAT | 360 |
| A2 | 2 | CGACTCAAGTGTGTGGAACATGGGGTCAGGGTTCTGGATAT | 361 |
| A3 | 3 | CGACTCAAGTGTGTGGAACGATGGGTCAGGGTTCTGGATAT | 362 |
| A4 | 4 | CGACTCAAGTGTGTGGAACGTAGGGTCAGGGTTCTGGATAT | 363 |
| A5 | 5 | CGACTCAAGTGTGTGGAACTAGGGGTCAGGGTTCTGGATAT | 364 |
| A6 | 6 | CGACTCAAGTGTGTGGAACTGAGGGTCAGGGTTCTGGATAT | 365 |
| A7 | 7 | CGACTCAAGTGTGTGGAAGACTGGGTCAGGGTTCTGGATAT | 366 |
| A8 | 8 | CGACTCAAGTGTGTGGAAGATCGGGTCAGGGTTCTGGATAT | 367 |
| A9 | 9 | CGACTCAAGTGTGTGGAAGCATGGGTCAGGGTTCTGGATAT | 368 |
| A10 | 10 | CGACTCAAGTGTGTGGAAGCTAGGGTCAGGGTTCTGGATAT | 369 |
| A11 | 11 | CGACTCAAGTGTGTGGAAGTACGGGTCAGGGTTCTGGATAT | 370 |
| A12 | 12 | CGACTCAAGTGTGTGGAAGTCAGGGTCAGGGTTCTGGATAT | 371 |
| B1 | 13 | CGACTCAAGTGTGTGGAATACGGGGTCAGGGTTCTGGATAT | 372 |
| B2 | 14 | CGACTCAAGTGTGTGGAATAGCGGGTCAGGGTTCTGGATAT | 373 |
| B3 | 15 | CGACTCAAGTGTGTGGAATCAGGGGTCAGGGTTCTGGATAT | 374 |
| B4 | 16 | CGACTCAAGTGTGTGGAATCGAGGGTCAGGGTTCTGGATAT | 375 |
| B5 | 17 | CGACTCAAGTGTGTGGAATGACGGGTCAGGGTTCTGGATAT | 376 |
| B6 | 18 | CGACTCAAGTGTGTGGAATGCAGGGTCAGGGTTCTGGATAT | 377 |
| B7 | 19 | CGACTCAAGTGTGTGGACAAGTGGGTCAGGGTTCTGGATAT | 378 |
| B8 | 20 | CGACTCAAGTGTGTGGACAATGGGGTCAGGGTTCTGGATAT | 379 |
| B9 | 21 | CGACTCAAGTGTGTGGACAGATGGGTCAGGGTTCTGGATAT | 380 |
| B10 | 22 | CGACTCAAGTGTGTGGACAGTAGGGTCAGGGTTCTGGATAT | 381 |
| B11 | 23 | CGACTCAAGTGTGTGGACATAGGGGTCAGGGTTCTGGATAT | 382 |
| B12 | 24 | CGACTCAAGTGTGTGGACATGAGGGTCAGGGTTCTGGATAT | 383 |
| C1 | 25 | CGACTCAAGTGTGTGGACCGCTGGGTCAGGGTTCTGGATAT | 384 |
| C2 | 26 | CGACTCAAGTGTGTGGACCGTCGGGTCAGGGTTCTGGATAT | 385 |
| C3 | 27 | CGACTCAAGTGTGTGGACCTCGGGGTCAGGGTTCTGGATAT | 386 |
| C4 | 28 | CGACTCAAGTGTGTGGACCTGCGGGTCAGGGTTCTGGATAT | 387 |
| C5 | 29 | CGACTCAAGTGTGTGGACGAATGGGTCAGGGTTCTGGATAT | 388 |
| C6 | 30 | CGACTCAAGTGTGTGGACGATAGGGTCAGGGTTCTGGATAT | 389 |
| C7 | 31 | CGACTCAAGTGTGTGGACGCCTGGGTCAGGGTTCTGGATAT | 390 |
| C8 | 32 | CGACTCAAGTGTGTGGACGCTCGGGTCAGGGTTCTGGATAT | 391 |
| C9 | 33 | CGACTCAAGTGTGTGGACGGTGGGGTCAGGGTTCTGGATAT | 392 |
| C10 | 34 | CGACTCAAGTGTGTGGACGTAAGGGTCAGGGTTCTGGATAT | 393 |
| C11 | 35 | CGACTCAAGTGTGTGGACGTCCGGGTCAGGGTTCTGGATAT | 394 |
| C12 | 36 | CGACTCAAGTGTGTGGACGTGGGGGTCAGGGTTCTGGATAT | 395 |
| D1 | 37 | CGACTCAAGTGTGTGGACTAAGGGGTCAGGGTTCTGGATAT | 396 |
| D2 | 38 | CGACTCAAGTGTGTGGACTAGAGGGTCAGGGTTCTGGATAT | 397 |
| D3 | 39 | CGACTCAAGTGTGTGGACTCCGGGGTCAGGGTTCTGGATAT | 398 |
| D4 | 40 | CGACTCAAGTGTGTGGACTCGCGGGTCAGGGTTCTGGATAT | 399 |
| D5 | 41 | CGACTCAAGTGTGTGGACTGAAGGGTCAGGGTTCTGGATAT | 400 |
| D6 | 42 | CGACTCAAGTGTGTGGACTGCCGGGTCAGGGTTCTGGATAT | 401 |
| D7 | 43 | CGACTCAAGTGTGTGGACTGTTGGGTCAGGGTTCTGGATAT | 402 |
| D8 | 44 | CGACTCAAGTGTGTGGACTTGTGGGTCAGGGTTCTGGATAT | 403 |
| D9 | 45 | CGACTCAAGTGTGTGGAGAACTGGGTCAGGGTTCTGGATAT | 404 |
| D10 | 46 | CGACTCAAGTGTGTGGAGAATCGGGTCAGGGTTCTGGATAT | 405 |
| D11 | 47 | CGACTCAAGTGTGTGGAGACATGGGTCAGGGTTCTGGATAT | 406 |
| D12 | 48 | CGACTCAAGTGTGTGGAGACTAGGGTCAGGGTTCTGGATAT | 407 |
| E1 | 49 | CGACTCAAGTGTGTGGAGATACGGGTCAGGGTTCTGGATAT | 408 |
| E2 | 50 | CGACTCAAGTGTGTGGAGATCAGGGTCAGGGTTCTGGATAT | 409 |
| E3 | 51 | CGACTCAAGTGTGTGGAGCAATGGGTCAGGGTTCTGGATAT | 410 |
| E4 | 52 | CGACTCAAGTGTGTGGAGCATAGGGTCAGGGTTCTGGATAT | 411 |
| E5 | 53 | CGACTCAAGTGTGTGGAGCCTCGGGTCAGGGTTCTGGATAT | 412 |
| E6 | 54 | CGACTCAAGTGTGTGGAGCGGTGGGTCAGGGTTCTGGATAT | 413 |
| E7 | 55 | CGACTCAAGTGTGTGGAGCGTGGGGTCAGGGTTCTGGATAT | 414 |
| E8 | 56 | CGACTCAAGTGTGTGGAGCTAAGGGTCAGGGTTCTGGATAT | 415 |
| E9 | 57 | CGACTCAAGTGTGTGGAGCTCCGGGTCAGGGTTCTGGATAT | 416 |
| E10 | 58 | CGACTCAAGTGTGTGGAGCTGGGGGTCAGGGTTCTGGATAT | 417 |
| E11 | 59 | CGACTCAAGTGTGTGGAGGCGTGGGTCAGGGTTCTGGATAT | 418 |
| E12 | 60 | CGACTCAAGTGTGTGGAGGCTGGGGTCAGGGTTCTGGATAT | 419 |
| F1 | 61 | CGACTCAAGTGTGTGGAGGTCGGGGTCAGGGTTCTGGATAT | 420 |
| F2 | 62 | CGACTCAAGTGTGTGGAGGTGCGGGTCAGGGTTCTGGATAT | 421 |
| F3 | 63 | CGACTCAAGTGTGTGGAGTAACGGGTCAGGGTTCTGGATAT | 422 |
| F4 | 64 | CGACTCAAGTGTGTGGAGTACAGGGTCAGGGTTCTGGATAT | 423 |
| F5 | 65 | CGACTCAAGTGTGTGGAGTCAAGGGTCAGGGTTCTGGATAT | 424 |
| F6 | 66 | CGACTCAAGTGTGTGGAGTCGGGGGTCAGGGTTCTGGATAT | 425 |
| F7 | 67 | CGACTCAAGTGTGTGGAGTCTTGGGTCAGGGTTCTGGATAT | 426 |
| F8 | 68 | CGACTCAAGTGTGTGGAGTGCGGGGTCAGGGTTCTGGATAT | 427 |
| F9 | 69 | CGACTCAAGTGTGTGGAGTGGCGGGTCAGGGTTCTGGATAT | 428 |
| F10 | 70 | CGACTCAAGTGTGTGGAGTTCTGGGTCAGGGTTCTGGATAT | 429 |
| F11 | 71 | CGACTCAAGTGTGTGGATAACGGGGTCAGGGTTCTGGATAT | 430 |
| F12 | 72 | CGACTCAAGTGTGTGGATAAGCGGGTCAGGGTTCTGGATAT | 431 |
| G1 | 73 | CGACTCAAGTGTGTGGATACAGGGGTCAGGGTTCTGGATAT | 432 |
| G2 | 74 | CGACTCAAGTGTGTGGATACGAGGGTCAGGGTTCTGGATAT | 433 |
| G3 | 75 | CGACTCAAGTGTGTGGATAGACGGGTCAGGGTTCTGGATAT | 434 |
| G4 | 76 | CGACTCAAGTGTGTGGATAGCAGGGTCAGGGTTCTGGATAT | 435 |
| G5 | 77 | CGACTCAAGTGTGTGGATCAAGGGGTCAGGGTTCTGGATAT | 436 |
| G6 | 78 | CGACTCAAGTGTGTGGATCAGAGGGTCAGGGTTCTGGATAT | 437 |
| G7 | 79 | CGACTCAAGTGTGTGGATCCGCGGGTCAGGGTTCTGGATAT | 438 |
| G8 | 80 | CGACTCAAGTGTGTGGATCGAAGGGTCAGGGTTCTGGATAT | 439 |
| G9 | 81 | CGACTCAAGTGTGTGGATCGCCGGGTCAGGGTTCTGGATAT | 440 |
| G10 | 82 | CGACTCAAGTGTGTGGATCGTTGGGTCAGGGTTCTGGATAT | 441 |
| G11 | 83 | CGACTCAAGTGTGTGGATCTGTGGGTCAGGGTTCTGGATAT | 442 |
| G12 | 84 | CGACTCAAGTGTGTGGATCTTGGGGTCAGGGTTCTGGATAT | 443 |
| H1 | 85 | CGACTCAAGTGTGTGGATGAACGGGTCAGGGTTCTGGATAT | 444 |
| H2 | 86 | CGACTCAAGTGTGTGGATGACAGGGTCAGGGTTCTGGATAT | 445 |
| H3 | 87 | CGACTCAAGTGTGTGGATGCAAGGGTCAGGGTTCTGGATAT | 446 |
| H4 | 88 | CGACTCAAGTGTGTGGATGCGGGGGTCAGGGTTCTGGATAT | 447 |
| H5 | 89 | CGACTCAAGTGTGTGGATGCTTGGGTCAGGGTTCTGGATAT | 448 |
| H6 | 90 | CGACTCAAGTGTGTGGATGGCGGGGTCAGGGTTCTGGATAT | 449 |
| H7 | 91 | CGACTCAAGTGTGTGGATGTCTGGGTCAGGGTTCTGGATAT | 450 |
| H8 | 92 | CGACTCAAGTGTGTGGATGTTCGGGTCAGGGTTCTGGATAT | 451 |
| H9 | 93 | CGACTCAAGTGTGTGGATTCGTGGGTCAGGGTTCTGGATAT | 452 |
| H10 | 94 | CGACTCAAGTGTGTGGATTCTGGGGTCAGGGTTCTGGATAT | 453 |
| H11 | 95 | CGACTCAAGTGTGTGGATTGCTGGGTCAGGGTTCTGGATAT | 454 |
| H12 | 96 | CGACTCAAGTGTGTGGATTGTCGGGTCAGGGTTCTGGATAT | 455 |

*Well-specific barcodes are in bold and C-segment-specific sequences are underlined.

TABLE 24

| Plate | Ind | MP_Hum_Boj (Human TCRβ reverse) Primer | SEQ ID |
|---|---|---|---|
| Well | # | sequence* (5′->3′) | NO: |
| A1 | 1 | CGACTCAGATTGGTACAACAGTACACSTTKTTCAGGTCCTC | 456 |
| A2 | 2 | CGACTCAGATTGGTACAACATGACACSTTKTTCAGGTCCTC | 457 |
| A3 | 3 | CGACTCAGATTGGTACAACGATACACSTTKTTCAGGTCCTC | 458 |
| A4 | 4 | CGACTCAGATTGGTACAACGTAACACSTTKTTCAGGTCCTC | 459 |
| A5 | 5 | CGACTCAGATTGGTACAACTAGACACSTTKTTCAGGTCCTC | 460 |
| A6 | 6 | CGACTCAGATTGGTACAACTGAACACSTTKTTCAGGTCCTC | 461 |
| A7 | 7 | CGACTCAGATTGGTACAAGACTACACSTTKTTCAGGTCCTC | 462 |
| A8 | 8 | CGACTCAGATTGGTACAAGATCACACSTTKTTCAGGTCCTC | 463 |
| A9 | 9 | CGACTCAGATTGGTACAAGCATACACSTTKTTCAGGTCCTC | 464 |
| A10 | 10 | CGACTCAGATTGGTACAAGCTAACACSTTKTTCAGGTCCTC | 465 |
| A11 | 11 | CGACTCAGATTGGTACAAGTACACACSTTKTTCAGGTCCTC | 466 |
| A12 | 12 | CGACTCAGATTGGTACAAGTCAACACSTTKTTCAGGTCCTC | 467 |
| B1 | 13 | CGACTCAGATTGGTACAATACGACACSTTKTTCAGGTCCTC | 468 |
| B2 | 14 | CGACTCAGATTGGTACAATAGCACACSTTKTTCAGGTCCTC | 469 |
| B3 | 15 | CGACTCAGATTGGTACAATCAGACACSTTKTTCAGGTCCTC | 470 |
| B4 | 16 | CGACTCAGATTGGTACAATCGAACACSTTKTTCAGGTCCTC | 471 |
| B5 | 17 | CGACTCAGATTGGTACAATGACACACSTTKTTCAGGTCCTC | 472 |
| B6 | 18 | CGACTCAGATTGGTACAATGCAACACSTTKTTCAGGTCCTC | 473 |
| B7 | 19 | CGACTCAGATTGGTACACAAGTACACSTTKTTCAGGTCCTC | 474 |
| B8 | 20 | CGACTCAGATTGGTACACAATGACACSTTKTTCAGGTCCTC | 475 |
| B9 | 21 | CGACTCAGATTGGTACACAGATACACSTTKTTCAGGTCCTC | 476 |
| B10 | 22 | CGACTCAGATTGGTACACAGTAACACSTTKTTCAGGTCCTC | 477 |
| B11 | 23 | CGACTCAGATTGGTACACATAGACACSTTKTTCAGGTCCTC | 478 |
| B12 | 24 | CGACTCAGATTGGTACACATGAACACSTTKTTCAGGTCCTC | 479 |
| C1 | 25 | CGACTCAGATTGGTACACCGCTACACSTTKTTCAGGTCCTC | 480 |
| C2 | 26 | CGACTCAGATTGGTACACCGTCACACSTTKTTCAGGTCCTC | 481 |
| C3 | 27 | CGACTCAGATTGGTACACCTCGACACSTTKTTCAGGTCCTC | 482 |
| C4 | 28 | CGACTCAGATTGGTACACCTGCACACSTTKTTCAGGTCCTC | 483 |
| C5 | 29 | CGACTCAGATTGGTACACGAATACACSTTKTTCAGGTCCTC | 484 |
| C6 | 30 | CGACTCAGATTGGTACACGATAACACSTTKTTCAGGTCCTC | 485 |
| C7 | 31 | CGACTCAGATTGGTACACGCCTACACSTTKTTCAGGTCCTC | 486 |
| C8 | 32 | CGACTCAGATTGGTACACGCTCACACSTTKTTCAGGTCCTC | 487 |
| C9 | 33 | CGACTCAGATTGGTACACGGTGACACSTTKTTCAGGTCCTC | 488 |
| C10 | 34 | CGACTCAGATTGGTACACGTAAACACSTTKTTCAGGTCCTC | 489 |
| C11 | 35 | CGACTCAGATTGGTACACGTCCACACSTTKTTCAGGTCCTC | 490 |
| C12 | 36 | CGACTCAGATTGGTACACGTGGACACSTTKTTCAGGTCCTC | 491 |
| D1 | 37 | CGACTCAGATTGGTACACTAAGACACSTTKTTCAGGTCCTC | 492 |
| D2 | 38 | CGACTCAGATTGGTACACTAGAACACSTTKTTCAGGTCCTC | 493 |
| D3 | 39 | CGACTCAGATTGGTACACTCCGACACSTTKTTCAGGTCCTC | 494 |
| D4 | 40 | CGACTCAGATTGGTACACTCGCACACSTTKTTCAGGTCCTC | 495 |
| D5 | 41 | CGACTCAGATTGGTACACTGAAACACSTTKTTCAGGTCCTC | 496 |
| D6 | 42 | CGACTCAGATTGGTACACTGCCACACSTTKTTCAGGTCCTC | 497 |
| D7 | 43 | CGACTCAGATTGGTACACTGTTACACSTTKTTCAGGTCCTC | 498 |
| D8 | 44 | CGACTCAGATTGGTACACTTGTACACSTTKTTCAGGTCCTC | 499 |
| D9 | 45 | CGACTCAGATTGGTACAGAACTACACSTTKTTCAGGTCCTC | 500 |
| D10 | 46 | CGACTCAGATTGGTACAGAATCACACSTTKTTCAGGTCCTC | 501 |
| D11 | 47 | CGACTCAGATTGGTACAGACATACACSTTKTTCAGGTCCTC | 502 |
| D12 | 48 | CGACTCAGATTGGTACAGACTAACACSTTKTTCAGGTCCTC | 503 |
| E1 | 49 | CGACTCAGATTGGTACAGATACACACSTTKTTCAGGTCCTC | 504 |
| E2 | 50 | CGACTCAGATTGGTACAGATCAACACSTTKTTCAGGTCCTC | 505 |
| E3 | 51 | CGACTCAGATTGGTACAGCAATACACSTTKTTCAGGTCCTC | 506 |
| E4 | 52 | CGACTCAGATTGGTACAGCATAACACSTTKTTCAGGTCCTC | 507 |
| E5 | 53 | CGACTCAGATTGGTACAGCCTCACACSTTKTTCAGGTCCTC | 508 |
| E6 | 54 | CGACTCAGATTGGTACAGCGGTACACSTTKTTCAGGTCCTC | 509 |
| E7 | 55 | CGACTCAGATTGGTACAGCGTGACACSTTKTTCAGGTCCTC | 510 |
| E8 | 56 | CGACTCAGATTGGTACAGCTAAACACSTTKTTCAGGTCCTC | 511 |
| E9 | 57 | CGACTCAGATTGGTACAGCTCCACACSTTKTTCAGGTCCTC | 512 |
| E10 | 58 | CGACTCAGATTGGTACAGCTGGACACSTTKTTCAGGTCCTC | 513 |
| E11 | 59 | CGACTCAGATTGGTACAGGCGTACACSTTKTTCAGGTCCTC | 514 |
| E12 | 60 | CGACTCAGATTGGTACAGGCTGACACSTTKTTCAGGTCCTC | 515 |
| F1 | 61 | CGACTCAGATTGGTACAGGTCGACACSTTKTTCAGGTCCTC | 516 |
| F2 | 62 | CGACTCAGATTGGTACAGGTGCACACSTTKTTCAGGTCCTC | 517 |
| F3 | 63 | CGACTCAGATTGGTACAGTAACACACSTTKTTCAGGTCCTC | 518 |
| F4 | 64 | CGACTCAGATTGGTACAGTACAACACSTTKTTCAGGTCCTC | 519 |
| F5 | 65 | CGACTCAGATTGGTACAGTCAAACACSTTKTTCAGGTCCTC | 520 |
| F6 | 66 | CGACTCAGATTGGTACAGTCGGACACSTTKTTCAGGTCCTC | 521 |
| F7 | 67 | CGACTCAGATTGGTACAGTCTTACACSTTKTTCAGGTCCTC | 522 |
| F8 | 68 | CGACTCAGATTGGTACAGTGCGACACSTTKTTCAGGTCCTC | 523 |
| F9 | 69 | CGACTCAGATTGGTACAGTGGCACACSTTKTTCAGGTCCTC | 524 |
| F10 | 70 | CGACTCAGATTGGTACAGTTCTACACSTTKTTCAGGTCCTC | 525 |
| F11 | 71 | CGACTCAGATTGGTACATAACGACACSTTKTTCAGGTCCTC | 526 |
| F12 | 72 | CGACTCAGATTGGTACATAAGCACACSTTKTTCAGGTCCTC | 527 |
| G1 | 73 | CGACTCAGATTGGTACATACAGACACSTTKTTCAGGTCCTC | 528 |
| G2 | 74 | CGACTCAGATTGGTACATACGAACACSTTKTTCAGGTCCTC | 529 |
| G3 | 75 | CGACTCAGATTGGTACATAGACACACSTTKTTCAGGTCCTC | 530 |
| G4 | 76 | CGACTCAGATTGGTACATAGCAACACSTTKTTCAGGTCCTC | 531 |
| G5 | 77 | CGACTCAGATTGGTACATCAAGACACSTTKTTCAGGTCCTC | 532 |
| G6 | 78 | CGACTCAGATTGGTACATCAGAACACSTTKTTCAGGTCCTC | 533 |
| G7 | 79 | CGACTCAGATTGGTACATCCGCACACSTTKTTCAGGTCCTC | 534 |
| G8 | 80 | CGACTCAGATTGGTACATCGAAACACSTTKTTCAGGTCCTC | 535 |
| G9 | 81 | CGACTCAGATTGGTACATCGCCACACSTTKTTCAGGTCCTC | 536 |
| G10 | 82 | CGACTCAGATTGGTACATCGTTACACSTTKTTCAGGTCCTC | 537 |
| G11 | 83 | CGACTCAGATTGGTACATCTGTACACSTTKTTCAGGTCCTC | 538 |
| G12 | 84 | CGACTCAGATTGGTACATCTTGACACSTTKTTCAGGTCCTC | 539 |
| H1 | 85 | CGACTCAGATTGGTACATGAACACACSTTKTTCAGGTCCTC | 540 |
| H2 | 86 | CGACTCAGATTGGTACATGACAACACSTTKTTCAGGTCCTC | 541 |
| H3 | 87 | CGACTCAGATTGGTACATGCAAACACSTTKTTCAGGTCCTC | 542 |
| H4 | 88 | CGACTCAGATTGGTACATGCGGACACSTTKTTCAGGTCCTC | 543 |
| H5 | 89 | CGACTCAGATTGGTACATGCTTACACSTTKTTCAGGTCCTC | 544 |
| H6 | 90 | CGACTCAGATTGGTACATGGCGACACSTTKTTCAGGTCCTC | 545 |
| H7 | 91 | CGACTCAGATTGGTACATGTCTACACSTTKTTCAGGTCCTC | 546 |
| H8 | 92 | CGACTCAGATTGGTACATGTTCACACSTTKTTCAGGTCCTC | 547 |
| H9 | 93 | CGACTCAGATTGGTACATTCGTACACSTTKTTCAGGTCCTC | 548 |
| H10 | 94 | CGACTCAGATTGGTACATTCTGACACSTTKTTCAGGTCCTC | 549 |
| H11 | 95 | CGACTCAGATTGGTACATTGCTACACSTTKTTCAGGTCCTC | 550 |
| H12 | 96 | CGACTCAGATTGGTACATTGTCACACSTTKTTCAGGTCCTC | 551 |

*Well-specific barcodes are in bold and C-segment-specific sequences are underlined.

TABLE 25

| Plate | Ind | MP_Mus_Acj (Mouse TCRα reverse) Primer | SEQ ID |
|---|---|---|---|
| Well | # | sequence* (5′->3′) | NO: |
| A1 | 1 | CGACTCAAGTGTGTGGAACAGTTTCTGGGTTCTGGATGT | 552 |
| A2 | 2 | CGACTCAAGTGTGTGGAACATGTTCTGGGTTCTGGATGT | 553 |
| A3 | 3 | CGACTCAAGTGTGTGGAACGATTTCTGGGTTCTGGATGT | 554 |
| A4 | 4 | CGACTCAAGTGTGTGGAACGTATTCTGGGTTCTGGATGT | 555 |
| A5 | 5 | CGACTCAAGTGTGTGGAACTAGTTCTGGGTTCTGGATGT | 556 |
| A6 | 6 | CGACTCAAGTGTGTGGAACTGATTCTGGGTTCTGGATGT | 557 |
| A7 | 7 | CGACTCAAGTGTGTGGAAGACTTTCTGGGTTCTGGATGT | 558 |
| A8 | 8 | CGACTCAAGTGTGTGGAAGATCTTCTGGGTTCTGGATGT | 559 |
| A9 | 9 | CGACTCAAGTGTGTGGAAGCATTTCTGGGTTCTGGATGT | 560 |
| A10 | 10 | CGACTCAAGTGTGTGGAAGCTATTCTGGGTTCTGGATGT | 561 |
| A11 | 11 | CGACTCAAGTGTGTGGAAGTACTTCTGGGTTCTGGATGT | 562 |
| A12 | 12 | CGACTCAAGTGTGTGGAAGTCATTCTGGGTTCTGGATGT | 563 |
| B1 | 13 | CGACTCAAGTGTGTGGAATACGTTCTGGGTTCTGGATGT | 564 |
| B2 | 14 | CGACTCAAGTGTGTGGAATAGCTTCTGGGTTCTGGATGT | 565 |
| B3 | 15 | CGACTCAAGTGTGTGGAATCAGTTCTGGGTTCTGGATGT | 566 |
| B4 | 16 | CGACTCAAGTGTGTGGAATCGATTCTGGGTTCTGGATGT | 567 |
| B5 | 17 | CGACTCAAGTGTGTGGAATGACTTCTGGGTTCTGGATGT | 568 |
| B6 | 18 | CGACTCAAGTGTGTGGAATGCATTCTGGGTTCTGGATGT | 569 |
| B7 | 19 | CGACTCAAGTGTGTGGACAAGTTTCTGGGTTCTGGATGT | 570 |
| B8 | 20 | CGACTCAAGTGTGTGGACAATGTTCTGGGTTCTGGATGT | 571 |
| B9 | 21 | CGACTCAAGTGTGTGGACAGATTTCTGGGTTCTGGATGT | 572 |
| B10 | 22 | CGACTCAAGTGTGTGGACAGTATTCTGGGTTCTGGATGT | 573 |
| B11 | 23 | CGACTCAAGTGTGTGGACATAGTTCTGGGTTCTGGATGT | 574 |
| B12 | 24 | CGACTCAAGTGTGTGGACATGATTCTGGGTTCTGGATGT | 575 |
| C1 | 25 | CGACTCAAGTGTGTGGACCGCTTTCTGGGTTCTGGATGT | 576 |
| C2 | 26 | CGACTCAAGTGTGTGGACCGTCTTCTGGGTTCTGGATGT | 577 |
| C3 | 27 | CGACTCAAGTGTGTGGACCTCGTTCTGGGTTCTGGATGT | 578 |
| C4 | 28 | CGACTCAAGTGTGTGGACCTGCTTCTGGGTTCTGGATGT | 579 |
| C5 | 29 | CGACTCAAGTGTGTGGACGAATTTCTGGGTTCTGGATGT | 580 |
| C6 | 30 | CGACTCAAGTGTGTGGACGATATTCTGGGTTCTGGATGT | 581 |
| C7 | 31 | CGACTCAAGTGTGTGGACGCCTTTCTGGGTTCTGGATGT | 582 |
| C8 | 32 | CGACTCAAGTGTGTGGACGCTCTTCTGGGTTCTGGATGT | 583 |
| C9 | 33 | CGACTCAAGTGTGTGGACGGTGTTCTGGGTTCTGGATGT | 584 |
| C10 | 34 | CGACTCAAGTGTGTGGACGTAATTCTGGGTTCTGGATGT | 585 |
| C11 | 35 | CGACTCAAGTGTGTGGACGTCCTTCTGGGTTCTGGATGT | 586 |
| C12 | 36 | CGACTCAAGTGTGTGGACGTGGTTCTGGGTTCTGGATGT | 587 |
| D1 | 37 | CGACTCAAGTGTGTGGACTAAGTTCTGGGTTCTGGATGT | 588 |
| D2 | 38 | CGACTCAAGTGTGTGGACTAGATTCTGGGTTCTGGATGT | 589 |
| D3 | 39 | CGACTCAAGTGTGTGGACTCCGTTCTGGGTTCTGGATGT | 590 |
| D4 | 40 | CGACTCAAGTGTGTGGACTCGCTTCTGGGTTCTGGATGT | 591 |
| D5 | 41 | CGACTCAAGTGTGTGGACTGAATTCTGGGTTCTGGATGT | 592 |
| D6 | 42 | CGACTCAAGTGTGTGGACTGCCTTCTGGGTTCTGGATGT | 593 |
| D7 | 43 | CGACTCAAGTGTGTGGACTGTTTTCTGGGTTCTGGATGT | 594 |
| D8 | 44 | CGACTCAAGTGTGTGGACTTGTTTCTGGGTTCTGGATGT | 595 |
| D9 | 45 | CGACTCAAGTGTGTGGAGAACTTTCTGGGTTCTGGATGT | 596 |
| D10 | 46 | CGACTCAAGTGTGTGGAGAATCTTCTGGGTTCTGGATGT | 597 |
| D11 | 47 | CGACTCAAGTGTGTGGAGACATTTCTGGGTTCTGGATGT | 598 |
| D12 | 48 | CGACTCAAGTGTGTGGAGACTATTCTGGGTTCTGGATGT | 599 |
| E1 | 49 | CGACTCAAGTGTGTGGAGATACTTCTGGGTTCTGGATGT | 600 |
| E2 | 50 | CGACTCAAGTGTGTGGAGATCATTCTGGGTTCTGGATGT | 601 |
| E3 | 51 | CGACTCAAGTGTGTGGAGCAATTTCTGGGTTCTGGATGT | 602 |
| E4 | 52 | CGACTCAAGTGTGTGGAGCATATTCTGGGTTCTGGATGT | 603 |
| E5 | 53 | CGACTCAAGTGTGTGGAGCCTCTTCTGGGTTCTGGATGT | 604 |
| E6 | 54 | CGACTCAAGTGTGTGGAGCGGTTTCTGGGTTCTGGATGT | 605 |
| E7 | 55 | CGACTCAAGTGTGTGGAGCGTGTTCTGGGTTCTGGATGT | 606 |
| E8 | 56 | CGACTCAAGTGTGTGGAGCTAATTCTGGGTTCTGGATGT | 607 |
| E9 | 57 | CGACTCAAGTGTGTGGAGCTCCTTCTGGGTTCTGGATGT | 608 |
| E10 | 58 | CGACTCAAGTGTGTGGAGCTGGTTCTGGGTTCTGGATGT | 609 |
| E11 | 59 | CGACTCAAGTGTGTGGAGGCGTTTCTGGGTTCTGGATGT | 610 |
| E12 | 60 | CGACTCAAGTGTGTGGAGGCTGTTCTGGGTTCTGGATGT | 611 |
| F1 | 61 | CGACTCAAGTGTGTGGAGGTCGTTCTGGGTTCTGGATGT | 612 |
| F2 | 62 | CGACTCAAGTGTGTGGAGGTGCTTCTGGGTTCTGGATGT | 613 |
| F3 | 63 | CGACTCAAGTGTGTGGAGTAACTTCTGGGTTCTGGATGT | 614 |
| F4 | 64 | CGACTCAAGTGTGTGGAGTACATTCTGGGTTCTGGATGT | 615 |
| F5 | 65 | CGACTCAAGTGTGTGGAGTCAATTCTGGGTTCTGGATGT | 616 |
| F6 | 66 | CGACTCAAGTGTGTGGAGTCGGTTCTGGGTTCTGGATGT | 617 |
| F7 | 67 | CGACTCAAGTGTGTGGAGTCTTTTCTGGGTTCTGGATGT | 618 |
| F8 | 68 | CGACTCAAGTGTGTGGAGTGCGTTCTGGGTTCTGGATGT | 619 |
| F9 | 69 | CGACTCAAGTGTGTGGAGTGGCTTCTGGGTTCTGGATGT | 620 |
| F10 | 70 | CGACTCAAGTGTGTGGAGTTCTTTCTGGGTTCTGGATGT | 621 |
| F11 | 71 | CGACTCAAGTGTGTGGATAACGTTCTGGGTTCTGGATGT | 622 |
| F12 | 72 | CGACTCAAGTGTGTGGATAAGCTTCTGGGTTCTGGATGT | 623 |
| G1 | 73 | CGACTCAAGTGTGTGGATACAGTTCTGGGTTCTGGATGT | 624 |
| G2 | 74 | CGACTCAAGTGTGTGGATACGATTCTGGGTTCTGGATGT | 625 |
| G3 | 75 | CGACTCAAGTGTGTGGATAGACTTCTGGGTTCTGGATGT | 626 |
| G4 | 76 | CGACTCAAGTGTGTGGATAGCATTCTGGGTTCTGGATGT | 627 |
| G5 | 77 | CGACTCAAGTGTGTGGATCAAGTTCTGGGTTCTGGATGT | 628 |
| G6 | 78 | CGACTCAAGTGTGTGGATCAGATTCTGGGTTCTGGATGT | 629 |
| G7 | 79 | CGACTCAAGTGTGTGGATCCGCTTCTGGGTTCTGGATGT | 630 |
| G8 | 80 | CGACTCAAGTGTGTGGATCGAATTCTGGGTTCTGGATGT | 631 |
| G9 | 81 | CGACTCAAGTGTGTGGATCGCCTTCTGGGTTCTGGATGT | 632 |
| G10 | 82 | CGACTCAAGTGTGTGGATCGTTTTCTGGGTTCTGGATGT | 633 |
| G11 | 83 | CGACTCAAGTGTGTGGATCTGTTTCTGGGTTCTGGATGT | 634 |
| G12 | 84 | CGACTCAAGTGTGTGGATCTTGTTCTGGGTTCTGGATGT | 635 |
| H1 | 85 | CGACTCAAGTGTGTGGATGAACTTCTGGGTTCTGGATGT | 636 |
| H2 | 86 | CGACTCAAGTGTGTGGATGACATTCTGGGTTCTGGATGT | 637 |
| H3 | 87 | CGACTCAAGTGTGTGGATGCAATTCTGGGTTCTGGATGT | 638 |
| H4 | 88 | CGACTCAAGTGTGTGGATGCGGTTCTGGGTTCTGGATGT | 639 |
| H5 | 89 | CGACTCAAGTGTGTGGATGCTTTTCTGGGTTCTGGATGT | 640 |
| H6 | 90 | CGACTCAAGTGTGTGGATGGCGTTCTGGGTTCTGGATGT | 641 |
| H7 | 91 | CGACTCAAGTGTGTGGATGTCTTTCTGGGTTCTGGATGT | 642 |
| H8 | 92 | CGACTCAAGTGTGTGGATGTTCTTCTGGGTTCTGGATGT | 643 |
| H9 | 93 | CGACTCAAGTGTGTGGATTCGTTTCTGGGTTCTGGATGT | 644 |
| H10 | 94 | CGACTCAAGTGTGTGGATTCTGTTCTGGGTTCTGGATGT | 645 |
| H11 | 95 | CGACTCAAGTGTGTGGATTGCTTTCTGGGTTCTGGATGT | 646 |
| H12 | 96 | CGACTCAAGTGTGTGGATTGTCTTCTGGGTTCTGGATGT | 647 |

*Well-specific barcodes are in bold and C-segment-specific sequences are underlined.

TABLE 26

| | | | SEQ |
|---|---|---|---|
| Plate | Ind | MP_Mus_Bcj (Mouse TCRB reverse) Primer | ID |
| Well | # | sequence* (5′->3′) | NO: |
| A1 | 1 | CGACTCAGATTGGTACAACAGTAGTCACATTTCTCAGATCCT | 648 |
| A2 | 2 | CGACTCAGATTGGTACAACATGAGTCACATTTCTCAGATCCT | 649 |
| A3 | 3 | CGACTCAGATTGGTACAACGATAGTCACATTTCTCAGATCCT | 650 |
| A4 | 4 | CGACTCAGATTGGTACAACGTAAGTCACATTTCTCAGATCCT | 651 |
| A5 | 5 | CGACTCAGATTGGTACAACTAGAGTCACATTTCTCAGATCCT | 652 |
| A6 | 6 | CGACTCAGATTGGTACAACTGAAGTCACATTTCTCAGATCCT | 653 |
| A7 | 7 | CGACTCAGATTGGTACAAGACTAGTCACATTTCTCAGATCCT | 654 |
| A8 | 8 | CGACTCAGATTGGTACAAGATCAGTCACATTTCTCAGATCCT | 655 |
| A9 | 9 | CGACTCAGATTGGTACAAGCATAGTCACATTTCTCAGATCCT | 656 |
| A10 | 10 | CGACTCAGATTGGTACAAGCTAAGTCACATTTCTCAGATCCT | 657 |
| A11 | 11 | CGACTCAGATTGGTACAAGTACAGTCACATTTCTCAGATCCT | 658 |
| A12 | 12 | CGACTCAGATTGGTACAAGTCAAGTCACATTTCTCAGATCCT | 659 |
| B1 | 13 | CGACTCAGATTGGTACAATACGAGTCACATTTCTCAGATCCT | 660 |
| B2 | 14 | CGACTCAGATTGGTACAATAGCAGTCACATTTCTCAGATCCT | 661 |
| B3 | 15 | CGACTCAGATTGGTACAATCAGAGTCACATTTCTCAGATCCT | 662 |
| B4 | 16 | CGACTCAGATTGGTACAATCGAAGTCACATTTCTCAGATCCT | 663 |
| B5 | 17 | CGACTCAGATTGGTACAATGACAGTCACATTTCTCAGATCCT | 664 |
| B6 | 18 | CGACTCAGATTGGTACAATGCAAGTCACATTTCTCAGATCCT | 665 |
| B7 | 19 | CGACTCAGATTGGTACACAAGTAGTCACATTTCTCAGATCCT | 666 |
| B8 | 20 | CGACTCAGATTGGTACACAATGAGTCACATTTCTCAGATCCT | 667 |
| B9 | 21 | CGACTCAGATTGGTACACAGATAGTCACATTTCTCAGATCCT | 668 |
| B10 | 22 | CGACTCAGATTGGTACACAGTAAGTCACATTTCTCAGATCCT | 669 |
| B11 | 23 | CGACTCAGATTGGTACACATAGAGTCACATTTCTCAGATCCT | 670 |
| B12 | 24 | CGACTCAGATTGGTACACATGAAGTCACATTTCTCAGATCCT | 671 |
| C1 | 25 | CGACTCAGATTGGTACACCGCTAGTCACATTTCTCAGATCCT | 672 |
| C2 | 26 | CGACTCAGATTGGTACACCGTCAGTCACATTTCTCAGATCCT | 673 |
| C3 | 27 | CGACTCAGATTGGTACACCTCGAGTCACATTTCTCAGATCCT | 674 |
| C4 | 28 | CGACTCAGATTGGTACACCTGCAGTCACATTTCTCAGATCCT | 675 |
| C5 | 29 | CGACTCAGATTGGTACACGAATAGTCACATTTCTCAGATCCT | 676 |
| C6 | 30 | CGACTCAGATTGGTACACGATAAGTCACATTTCTCAGATCCT | 677 |
| C7 | 31 | CGACTCAGATTGGTACACGCCTAGTCACATTTCTCAGATCCT | 678 |
| C8 | 32 | CGACTCAGATTGGTACACGCTCAGTCACATTTCTCAGATCCT | 679 |
| C9 | 33 | CGACTCAGATTGGTACACGGTGAGTCACATTTCTCAGATCCT | 680 |
| C10 | 34 | CGACTCAGATTGGTACACGTAAAGTCACATTTCTCAGATCCT | 681 |
| C11 | 35 | CGACTCAGATTGGTACACGTCCAGTCACATTTCTCAGATCCT | 682 |
| C12 | 36 | CGACTCAGATTGGTACACGTGGAGTCACATTTCTCAGATCCT | 683 |
| D1 | 37 | CGACTCAGATTGGTACACTAAGAGTCACATTTCTCAGATCCT | 684 |
| D2 | 38 | CGACTCAGATTGGTACACTAGAAGTCACATTTCTCAGATCCT | 685 |
| D3 | 39 | CGACTCAGATTGGTACACTCCGAGTCACATTTCTCAGATCCT | 686 |
| D4 | 40 | CGACTCAGATTGGTACACTCGCAGTCACATTTCTCAGATCCT | 687 |
| D5 | 41 | CGACTCAGATTGGTACACTGAAAGTCACATTTCTCAGATCCT | 688 |
| D6 | 42 | CGACTCAGATTGGTACACTGCCAGTCACATTTCTCAGATCCT | 689 |
| D7 | 43 | CGACTCAGATTGGTACACTGTTAGTCACATTTCTCAGATCCT | 690 |
| D8 | 44 | CGACTCAGATTGGTACACTTGTAGTCACATTTCTCAGATCCT | 691 |
| D9 | 45 | CGACTCAGATTGGTACAGAACTAGTCACATTTCTCAGATCCT | 692 |
| D10 | 46 | CGACTCAGATTGGTACAGAATCAGTCACATTTCTCAGATCCT | 693 |
| D11 | 47 | CGACTCAGATTGGTACAGACATAGTCACATTTCTCAGATCCT | 694 |
| D12 | 48 | CGACTCAGATTGGTACAGACTAAGTCACATTTCTCAGATCCT | 695 |
| E1 | 49 | CGACTCAGATTGGTACAGATACAGTCACATTTCTCAGATCCT | 696 |
| E2 | 50 | CGACTCAGATTGGTACAGATCAAGTCACATTTCTCAGATCCT | 697 |
| E3 | 51 | CGACTCAGATTGGTACAGCAATAGTCACATTTCTCAGATCCT | 698 |
| E4 | 52 | CGACTCAGATTGGTACAGCATAAGTCACATTTCTCAGATCCT | 699 |
| E5 | 53 | CGACTCAGATTGGTACAGCCTCAGTCACATTTCTCAGATCCT | 700 |
| E6 | 54 | CGACTCAGATTGGTACAGCGGTAGTCACATTTCTCAGATCCT | 701 |
| E7 | 55 | CGACTCAGATTGGTACAGCGTGAGTCACATTTCTCAGATCCT | 702 |
| E8 | 56 | CGACTCAGATTGGTACAGCTAAAGTCACATTTCTCAGATCCT | 703 |
| E9 | 57 | CGACTCAGATTGGTACAGCTCCAGTCACATTTCTCAGATCCT | 704 |
| E10 | 58 | CGACTCAGATTGGTACAGCTGGAGTCACATTTCTCAGATCCT | 705 |
| E11 | 59 | CGACTCAGATTGGTACAGGCGTAGTCACATTTCTCAGATCCT | 706 |
| E12 | 60 | CGACTCAGATTGGTACAGGCTGAGTCACATTTCTCAGATCCT | 707 |
| F1 | 61 | CGACTCAGATTGGTACAGGTCGAGTCACATTTCTCAGATCCT | 708 |
| F2 | 62 | CGACTCAGATTGGTACAGGTGCAGTCACATTTCTCAGATCCT | 709 |
| F3 | 63 | CGACTCAGATTGGTACAGTAACAGTCACATTTCTCAGATCCT | 710 |
| F4 | 64 | CGACTCAGATTGGTACAGTACAAGTCACATTTCTCAGATCCT | 711 |
| F5 | 65 | CGACTCAGATTGGTACAGTCAAAGTCACATTTCTCAGATCCT | 712 |
| F6 | 66 | CGACTCAGATTGGTACAGTCGGAGTCACATTTCTCAGATCCT | 713 |
| F7 | 67 | CGACTCAGATTGGTACAGTCTTAGTCACATTTCTCAGATCCT | 714 |
| F8 | 68 | CGACTCAGATTGGTACAGTGCGAGTCACATTTCTCAGATCCT | 715 |
| F9 | 69 | CGACTCAGATTGGTACAGTGGCAGTCACATTTCTCAGATCCT | 716 |
| F10 | 70 | CGACTCAGATTGGTACAGTTCTAGTCACATTTCTCAGATCCT | 717 |
| F11 | 71 | CGACTCAGATTGGTACATAACGAGTCACATTTCTCAGATCCT | 718 |
| F12 | 72 | CGACTCAGATTGGTACATAAGCAGTCACATTTCTCAGATCCT | 719 |
| G1 | 73 | CGACTCAGATTGGTACATACAGAGTCACATTTCTCAGATCCT | 720 |
| G2 | 74 | CGACTCAGATTGGTACATACGAAGTCACATTTCTCAGATCCT | 721 |
| G3 | 75 | CGACTCAGATTGGTACATAGACAGTCACATTTCTCAGATCCT | 722 |
| G4 | 76 | CGACTCAGATTGGTACATAGCAAGTCACATTTCTCAGATCCT | 723 |
| G5 | 77 | CGACTCAGATTGGTACATCAAGAGTCACATTTCTCAGATCCT | 724 |
| G6 | 78 | CGACTCAGATTGGTACATCAGAAGTCACATTTCTCAGATCCT | 725 |
| G7 | 79 | CGACTCAGATTGGTACATCCGCAGTCACATTTCTCAGATCCT | 726 |
| G8 | 80 | CGACTCAGATTGGTACATCGAAAGTCACATTTCTCAGATCCT | 727 |
| G9 | 81 | CGACTCAGATTGGTACATCGCCAGTCACATTTCTCAGATCCT | 728 |
| G10 | 82 | CGACTCAGATTGGTACATCGTTAGTCACATTTCTCAGATCCT | 729 |
| G11 | 83 | CGACTCAGATTGGTACATCTGTAGTCACATTTCTCAGATCCT | 730 |
| G12 | 84 | CGACTCAGATTGGTACATCTTGAGTCACATTTCTCAGATCCT | 731 |
| H1 | 85 | CGACTCAGATTGGTACATGAACAGTCACATTTCTCAGATCCT | 732 |
| H2 | 86 | CGACTCAGATTGGTACATGACAAGTCACATTTCTCAGATCCT | 733 |
| H3 | 87 | CGACTCAGATTGGTACATGCAAAGTCACATTTCTCAGATCCT | 734 |
| H4 | 88 | CGACTCAGATTGGTACATGCGGAGTCACATTTCTCAGATCCT | 735 |
| H5 | 89 | CGACTCAGATTGGTACATGCTTAGTCACATTTCTCAGATCCT | 736 |
| H6 | 90 | CGACTCAGATTGGTACATGGCGAGTCACATTTCTCAGATCCT | 737 |
| H7 | 91 | CGACTCAGATTGGTACATGTCTAGTCACATTTCTCAGATCCT | 738 |
| H8 | 92 | CGACTCAGATTGGTACATGTTCAGTCACATTTCTCAGATCCT | 739 |
| H9 | 93 | CGACTCAGATTGGTACATTCGTAGTCACATTTCTCAGATCCT | 740 |
| H10 | 94 | CGACTCAGATTGGTACATTCTGAGTCACATTTCTCAGATCCT | 741 |
| H11 | 95 | CGACTCAGATTGGTACATTGCTAGTCACATTTCTCAGATCCT | 742 |
| H12 | 96 | CGACTCAGATTGGTACATTGTCAGTCACATTTCTCAGATCCT | 743 |

*Well-specific barcodes are in bold and C-segment-specific sequences are underlined.

To each well of the alpha and beta plates is added 9 μl of the master mix and 1 μl of the amplicons from the first round. The plate is resealed and centrifuged to bring the contents to the bottom of the wells. PCR of human TCRαβ is carried out in a thermocycler as follows: initial denaturation at 95° C. for 5 minutes; 24 cycles of denaturation at 95° C. for 20 seconds, primer annealing at 52° C. for 20 seconds, polymerase extension at 72° for 45 seconds; a final extension at 72° C. for 7 minutes and a final hold at 4° C. PCR of mouse TCRαβ is carried out as described for human TCRαβ except that primer annealing is carried out at 55° C. instead of 52° C. Samples in the plates are stored at −20° C.

PCR Product Pooling and Purification. PCR products (2 μl from each well) of the second round of amplification were pooled and purified with AMPURE® XP beads (Beckman Coulter) according manufacturer's protocol at a 1:1.2 sample:beads ratio. Briefly, the beads and sample were mixed, incubated for 15 minutes, and placed in a magnet for 3 minutes. The supernatant was subsequently discarded, the sample was washed twice with 200 μl 80% ethanol for 30 seconds each wash. The samples were air dried, resuspended in 50 μl water, incubated at 50° C. for 5 minutes, placed into the magnet for 5 minutes and the supernatant containing the purified PCR products was transferred to a new tube.

Sequencing. The concentration of the purified PCR products was determined using a QUBIT® High Sensitivity DNA kit (Invitrogen). Adapters necessary for ILLUMINA® platform sequencing were ligated to the PCR products using KAPA HyperPrep Kit (Roche) according to the manufacturer's protocol. After ligation of the adapter, the library of amplified TCRs is sequenced on an ILLUMINA® MISEQ®, HISEO® or NOVASEQ® platform 2×150 paired read length, 500 thousand reads per library.

### Example 3: TCR Analysis of an Immunized Mice

CD3+CD8+OT-1-tetramer positive cells were isolated from an immunized mouse and sorted into a 384-well microtiter plate, one cell per well. Cells were not sorted into column 12 of the microtiter plate as this column provided a negative control. Plates underwent cDNA synthesis and murine TCRαβ amplification in accordance with the method of this invention using the primers in Tables 9, 10, 13 and 14. Sequencing was carried out on an Illumina NovaSeq platform.

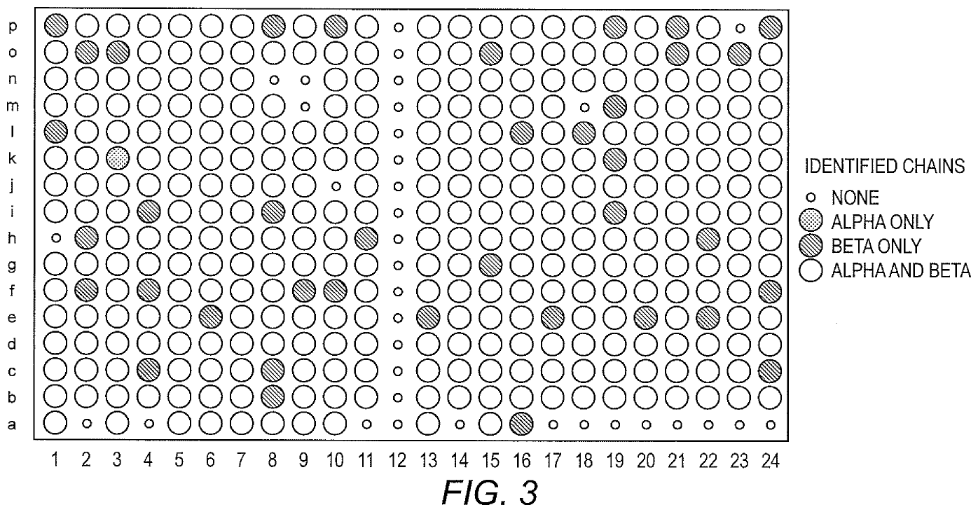

The results of this analysis (FIG. 3) indicated that a TCRβ chain and/or TCRα chain was detected in a substantial number of cells. Sequencing of the CDR3 sequence indicated the extent of V(D)J junctional diversity (Table 27).

TABLE 27

| | | | | | Well | |
|---|---|---|---|---|---|---|
| Barcode | BestV_β | BestJ_β | BestV_α | BestJ_α | x | y |
| 1 | TRBV16 | TRBJ2-4 | TRAV16D-DV11 | TRAJ26 | a | 1 |
| 2 | TRBV2 | TRBJ2-5 | TRAV6-5 | TRAJ23 | a | 3 |
| 3 | TRBV13-1 | TRBJ1-5 | TRAV7-4 | TRAJ40 | a | 5 |
| 4 | TRBV12-1 | TRBJ1-1 | TRAV6N-5 | TRAJ12 | a | 7 |
| 5 | TRBV13-3 | TRBJ2-2 | TRAV6-5 | TRAJ23 | a | 9 |
| 6 | NA | NA | NA | NA | a | 11 |
| 7 | TRBV2 | TRBJ2-5 | TRAV6-5 | TRAJ23 | a | 13 |
| 8 | TRBV13-3 | TRBJ2-2 | TRAV6-5 | TRAJ23 | a | 15 |
| 9 | NA | NA | NA | NA | a | 17 |
| 10 | NA | NA | NA | NA | a | 19 |
| 11 | NA | NA | NA | NA | a | 21 |
| 12 | NA | NA | NA | NA | a | 23 |
| 13 | TRBV12-1 | TRBJ1-1 | TRAV6N-5 | TRAJ12 | c | 1 |
| 14 | TRBV16 | TRBJ2-4 | TRAV16D-DV11 | TRAJ26 | c | 3 |
| 15 | TRBV13-2 | TRBJ1-5 | TRAV6-5 | TRAJ27 | c | 5 |
| 16 | TRBV13-3 | TRBJ2-2 | TRAV6-5 | TRAJ23 | c | 7 |
| 17 | TRBV13-3 | TRBJ2-2 | TRAV6-5 | TRAJ23 | c | 9 |
| 18 | TRBV12-1 | TRBJ2-7 | TRAV14D-3-DV8 | TRAJ26 | c | 11 |
| 19 | TRBV16 | TRBJ2-4 | TRAV16D-DV11 | TRAJ26 | c | 13 |
| 20 | TRBV13-1 | TRBJ1-5 | TRAV7-4 | TRAJ40 | c | 15 |
| 21 | TRBV12-1 | TRBJ1-2 | TRAV9N-3 | TRAJ12 | c | 17 |
| 22 | TRBV13-2 | TRBJ2-3 | TRAV10 | TRAJ22 | c | 19 |
| 23 | TRBV13-3 | TRBJ2-2 | TRAV6-5 | TRAJ23 | c | 21 |
| 24 | TRBV31 | TRBJ2-7 | TRAV7-1 | TRAJ32 | c | 23 |
| 25 | TRBV12-1 | TRBJ2-7 | TRAV7-2 | TRAJ30 | e | 1 |
| 26 | TRBV12-1 | TRBJ1-1 | TRAV6N-5 | TRAJ12 | e | 3 |
| 27 | TRBV12-1 | TRBJ1-1 | TRAV6N-5 | TRAJ12 | e | 5 |
| 28 | TRBV13-3 | TRBJ2-2 | TRAV6-5 | TRAJ23 | e | 7 |
| 29 | TRBV2 | TRBJ2-5 | TRAV6-5 | TRAJ23 | e | 9 |
| 30 | TRBV12-1 | TRBJ1-1 | TRAV6N-5 | TRAJ12 | e | 11 |
| 31 | TRBV12-1 | TRBJ1-2 | NA | NA | e | 13 |
| 32 | TRBV13-1 | TRBJ2-5 | TRAV12-2 | TRAJ21 | e | 15 |
| 33 | TRBV13-2 | TRBJ2-3 | NA | NA | e | 17 |
| 34 | TRBV12-1 | TRBJ2-7 | TRAV9-1 | TRAJ12 | e | 19 |
| 35 | TRBV12-1 | TRBJ1-1 | TRAV6N-5 | TRAJ12 | e | 21 |
| 36 | TRBV13-2 | TRBJ1-5 | TRAV6-5 | TRAJ27 | e | 23 |
| 37 | TRBV13-3 | TRBJ1-1 | TRAV3-3 | TRAJ43 | g | 1 |
| 38 | TRBV31 | TRBJ2-1 | TRAV12-1 | TRAJ27 | g | 3 |
| 39 | TRBV2 | TRBJ2-5 | TRAV6-5 | TRAJ23 | g | 5 |
| 40 | TRBV13-3 | TRBJ2-2 | TRAV6-5 | TRAJ23 | g | 7 |
| 41 | TRBV13-3 | TRBJ2-2 | TRAV6-5 | TRAJ23 | g | 9 |
| 42 | TRBV16 | TRBJ2-4 | TRAV16D-DV11 | TRAJ26 | g | 11 |
| 43 | TRBV13-3 | TRBJ2-2 | TRAV6-5 | TRAJ23 | g | 13 |
| 44 | TRBV13-1 | TRBJ2-5 | NA | NA | g | 15 |
| 45 | TRBV13-3 | TRBJ2-2 | TRAV6-5 | TRAJ23 | g | 17 |
| 46 | TRBV13-3 | TRBJ2-2 | TRAV6-5 | TRAJ23 | g | 19 |
| 47 | TRBV13-3 | TRBJ2-2 | TRAV6-5 | TRAJ23 | g | 21 |
| 48 | TRBV13-3 | TRBJ2-2 | TRAV6-5 | TRAJ23 | i | 23 |
| 49 | TRBV12-1 | TRBJ2-2 | TRAV9D-3 | TRAJ2 | i | 1 |
| 50 | TRBV13-2 | TRBJ1-5 | TRAV6-5 | TRAJ27 | i | 3 |
| 51 | TRBV2 | TRBJ2-5 | TRAV6-5 | TRAJ23 | i | 5 |
| 52 | TRBV12-1 | TRBJ1-1 | TRAV6N-5 | TRAJ12 | i | 7 |
| 53 | TRBV16 | TRBJ2-4 | TRAV16D-DV11 | TRAJ26 | i | 9 |
| 54 | TRBV13-3 | TRBJ2-2 | TRAV6-5 | TRAJ23 | i | 11 |
| 55 | TRBV12-1 | TRBJ1-1 | TRAV6N-5 | TRAJ12 | i | 13 |
| 56 | TRBV31 | TRBJ2-1 | TRAV12-1 | TRAJ27 | i | 15 |
| 57 | TRBV13-3 | TRBJ2-2 | TRAV6-5 | TRAJ23 | i | 17 |
| 58 | TRBV12-1 | TRBJ1-2 | NA | NA | i | 19 |
| 59 | TRBV13-3 | TRBJ2-2 | TRAV6-5 | TRAJ23 | i | 21 |
| 60 | TRBV13-2 | TRBJ1-5 | TRAV6-5 | TRAJ27 | i | 23 |
| 61 | TRBV13-3 | TRBJ2-2 | TRAV6-5 | TRAJ23 | k | 1 |
| 62 | NA | NA | TRAV6-5 | TRAJ23 | k | 3 |
| 63 | TRBV12-1 | TRBJ1-2 | TRAV6-1 | TRAJ12 | k | 5 |
| 64 | TRBV13-2 | TRBJ1-5 | TRAV6-5 | TRAJ27 | k | 7 |
| 65 | TRBV13-3 | TRBJ2-2 | TRAV6-5 | TRAJ23 | k | 9 |
| 66 | TRBV13-2 | TRBJ1-5 | TRAV6-5 | TRAJ27 | k | 11 |
| 67 | TRBV13-3 | TRBJ2-2 | TRAV6-5 | TRAJ23 | k | 13 |
| 68 | TRBV29 | TRBJ2-1 | TRAV3D-3 | TRAJ15 | k | 15 |
| 69 | TRBV13-3 | TRBJ2-2 | TRAV6-5 | TRAJ23 | k | 17 |
| 70 | TRBV31 | TRBJ2-1 | NA | NA | k | 19 |
| 71 | TRBV12-1 | TRBJ2-2 | TRAV9D-3 | TRAJ2 | k | 21 |
| 72 | TRBV12-1 | TRBJ1-1 | TRAV6N-5 | TRAJ12 | k | 23 |
| 73 | TRBV16 | TRBJ2-4 | TRAV16D-DV11 | TRAJ26 | m | 1 |
| 74 | TRBV13-1 | TRBJ2-5 | TRAV12-2 | TRAJ21 | m | 3 |
| 75 | TRBV16 | TRBJ2-4 | TRAV16D-DV11 | TRAJ26 | m | 5 |
| 76 | TRBV16 | TRBJ2-4 | TRAV16D-DV11 | TRAJ26 | m | 7 |
| 77 | NA | NA | NA | NA | m | 9 |
| 78 | TRBV12-1 | TRBJ2-2 | TRAV9D-3 | TRAJ2 | m | 11 |
| 79 | TRBV13-2 | TRBJ2-3 | TRAV10 | TRAJ22 | m | 13 |
| 80 | TRBV13-3 | TRBJ2-2 | TRAV6-5 | TRAJ23 | m | 15 |
| 81 | TRBV13-3 | TRBJ2-2 | TRAV6-5 | TRAJ23 | m | 17 |
| 82 | TRBV13-3 | TRBJ1-1 | NA | NA | m | 19 |
| 83 | TRBV13-1 | TRBJ1-5 | TRAV7-4 | TRAJ40 | m | 21 |
| 84 | TRBV13-1 | TRBJ1-5 | TRAV7-4 | TRAJ40 | m | 23 |
| 85 | TRBV12-1 | TRBJ1-1 | TRAV6-6 | TRAJ12 | o | 1 |
| 86 | TRBV16 | TRBJ2-4 | NA | NA | o | 3 |
| 87 | TRBV12-1 | TRBJ1-1 | TRAV6-6 | TRAJ12 | o | 5 |
| 88 | TRBV12-1 | TRBJ1-1 | TRAV6-6 | TRAJ12 | o | 7 |
| 89 | TRBV13-1 | TRBJ2-7 | TRAV14D-3-DV8 | TRAJ11 | o | 9 |
| 90 | TRBV13-2 | TRBJ2-3 | TRAV10 | TRAJ22 | o | 11 |
| 91 | TRBV16 | TRBJ2-4 | TRAV16D-DV11 | TRAJ26 | o | 13 |
| 92 | TRBV16 | TRBJ2-4 | NA | NA | o | 15 |
| 93 | TRBV12-1 | TRBJ2-2 | TRAV9D-3 | TRAJ2 | o | 17 |
| 94 | TRBV13-3 | TRBJ2-2 | TRAV6-5 | TRAJ23 | o | 19 |
| 95 | TRBV31 | TRBJ2-1 | NA | NA | o | 21 |
| 96 | TRBV31 | TRBJ2-1 | NA | NA | o | 23 |
| 1 | NA | NA | NA | NA | a | 2 |
| 2 | NA | NA | NA | NA | a | 4 |
| 3 | TRBV13-3 | TRBJ2-2 | TRAV6-5 | TRAJ23 | a | 6 |
| 4 | TRBV13-3 | TRBJ2-2 | TRAV6-5 | TRAJ23 | a | 8 |
| 5 | TRBV31 | TRBJ2-7 | TRAV7-1 | TRAJ32 | a | 10 |
| 6 | NA | NA | NA | NA | a | 12 |
| 7 | NA | NA | NA | NA | a | 14 |
| 8 | TRBV16 | TRBJ2-4 | NA | NA | a | 16 |
| 9 | NA | NA | NA | NA | a | 18 |
| 10 | NA | NA | NA | NA | a | 20 |
| 11 | NA | NA | NA | NA | a | 22 |
| 12 | NA | NA | NA | NA | a | 24 |
| 13 | TRBV13-2 | TRBJ2-3 | TRAV10 | TRAJ22 | c | 2 |
| 14 | TRBV19 | TRBJ1-6 | NA | NA | c | 4 |
| 15 | TRBV12-1 | TRBJ1-1 | TRAV6N-5 | TRAJ12 | c | 6 |
| 16 | TRBV31 | TRBJ2-1 | NA | NA | c | 8 |
| 17 | TRBV13-2 | TRBJ2-3 | TRAV10 | TRAJ22 | c | 10 |
| 18 | NA | NA | NA | NA | c | 12 |
| 19 | TRBV13-2 | TRBJ2-3 | TRAV10 | TRAJ22 | c | 14 |
| 20 | TRBV13-3 | TRBJ2-2 | TRAV6-5 | TRAJ23 | c | 16 |
| 21 | TRBV13-3 | TRBJ2-2 | TRAV6-5 | TRAJ23 | c | 18 |
| 22 | TRBV13-3 | TRBJ2-2 | TRAV6-5 | TRAJ23 | c | 20 |
| 23 | TRBV12-1 | TRBJ1-2 | TRAV6-1 | TRAJ12 | c | 22 |
| 24 | TRBV19 | TRBJ1-6 | NA | NA | c | 24 |
| 25 | TRBV12-1 | TRBJ1-1 | TRAV6N-5 | TRAJ12 | e | 2 |
| 26 | TRBV13-3 | TRBJ2-2 | TRAV6-5 | TRAJ23 | e | 4 |
| 27 | TRBV31 | TRBJ2-1 | NA | NA | e | 6 |
| 28 | TRBV31 | TRBJ2-2 | TRAV14D-2 | TRAJ16 | e | 8 |
| 29 | TRBV13-2 | TRBJ2-2 | TRAV6-5 | TRAJ30 | e | 10 |
| 30 | NA | NA | NA | NA | e | 12 |
| 31 | TRBV13-2 | TRBJ2-3 | TRAV10 | TRAJ22 | e | 14 |
| 32 | TRBV16 | TRBJ2-4 | TRAV16D-DV11 | TRAJ26 | e | 16 |
| 33 | TRBV2 | TRBJ2-5 | TRAV6-5 | TRAJ23 | e | 18 |
| 34 | TRBV31 | TRBJ2-2 | NA | NA | e | 20 |
| 35 | TRBV31 | TRBJ2-1 | NA | NA | e | 22 |
| 36 | TRBV12-1 | TRBJ2-2 | TRAV9D-3 | TRAJ2 | e | 24 |
| 37 | TRBV16 | TRBJ2-4 | TRAV16D-DV11 | TRAJ26 | g | 2 |
| 38 | TRBV12-1 | TRBJ1-1 | TRAV6N-5 | TRAJ12 | g | 4 |
| 39 | TRBV13-3 | TRBJ2-2 | TRAV6-5 | TRAJ23 | g | 6 |
| 40 | TRBV13-2 | TRBJ2-2 | TRAV6-5 | TRAJ30 | g | 8 |
| 41 | TRBV13-3 | TRBJ2-2 | TRAV6-5 | TRAJ23 | g | 10 |
| 42 | NA | NA | NA | NA | g | 12 |
| 43 | TRBV12-1 | TRBJ1-2 | TRAV6-6 | TRAJ12 | g | 14 |
| 44 | TRBV12-1 | TRBJ1-2 | TRAV6-1 | TRAJ12 | g | 16 |
| 45 | TRBV12-1 | TRBJ1-1 | TRAV6N-5 | TRAJ12 | g | 18 |
| 46 | TRBV13-3 | TRBJ2-2 | TRAV6-5 | TRAJ23 | g | 20 |
| 47 | TRBV12-1 | TRBJ1-1 | TRAV6N-5 | TRAJ12 | g | 22 |
| 48 | TRBV29 | TRBJ1-5 | TRAV6-5 | TRAJ44 | g | 24 |
| 49 | TRBV16 | TRBJ2-4 | TRAV16D-DV11 | TRAJ26 | i | 2 |
| 50 | TRBV12-1 | TRBJ1-2 | NA | NA | i | 4 |
| 51 | TRBV12-1 | TRBJ1-2 | TRAV6N-5 | TRAJ12 | i | 6 |
| 52 | TRBV19 | TRBJ1-6 | NA | NA | i | 8 |
| 53 | TRBV13-1 | TRBJ2-5 | TRAV12-2 | TRAJ21 | i | 10 |
| 54 | NA | NA | NA | NA | i | 12 |
| 55 | TRBV2 | TRBJ2-5 | TRAV6-5 | TRAJ23 | i | 14 |
| 56 | TRBV13-3 | TRBJ2-2 | TRAV6-5 | TRAJ23 | i | 16 |
| 57 | TRBV12-1 | TRBJ2-7 | TRAV7-2 | TRAJ30 | i | 18 |
| 58 | TRBV13-2 | TRBJ2-3 | TRAV10 | TRAJ22 | i | 20 |
| 59 | TRBV29 | TRBJ1-5 | TRAV6-5 | TRAJ44 | i | 22 |
| 60 | TRBV12-1 | TRBJ1-2 | TRAV6N-5 | TRAJ12 | i | 24 |
| 61 | TRBV13-3 | TRBJ2-2 | TRAV6-5 | TRAJ23 | k | 2 |
| 62 | TRBV31 | TRBJ2-2 | TRAV14D-2 | TRAJ16 | k | 4 |
| 63 | TRBV13-2 | TRBJ2-3 | TRAV10 | TRAJ22 | k | 6 |
| 64 | TRBV12-1 | TRBJ1-2 | TRAV6-6 | TRAJ12 | k | 8 |
| 65 | TRBV13-2 | TRBJ1-5 | TRAV6-5 | TRAJ27 | k | 10 |
| 66 | NA | NA | NA | NA | k | 12 |
| 67 | TRBV31 | TRBJ2-7 | TRAV7-1 | TRAJ32 | k | 14 |
| 68 | TRBV2 | TRBJ2-5 | TRAV6-5 | TRAJ23 | k | 16 |
| 69 | TRBV12-1 | TRBJ2-2 | TRAV9D-3 | TRAJ2 | k | 18 |
| 70 | TRBV12-1 | TRBJ1-1 | TRAV6N-5 | TRAJ12 | k | 20 |
| 71 | TRBV12-1 | TRBJ2-5 | TRAV14N-3 | TRAJ31 | k | 22 |
| 72 | TRBV14 | TRBJ2-7 | TRAV6-7-DV9 | TRAJ49 | k | 24 |
| 73 | TRBV12-1 | TRBJ2-2 | TRAV9D-3 | TRAJ2 | m | 2 |
| 74 | TRBV13-2 | TRBJ2-3 | TRAV10 | TRAJ22 | m | 4 |
| 75 | TRBV13-3 | TRBJ2-2 | TRAV6-5 | TRAJ23 | m | 6 |
| 76 | TRBV13-3 | TRBJ2-2 | TRAV6-5 | TRAJ23 | m | 8 |
| 77 | TRBV12-1 | TRBJ2-7 | TRAV9-1 | TRAJ12 | m | 10 |
| 78 | NA | NA | NA | NA | m | 12 |
| 79 | TRBV12-1 | TRBJ2-7 | TRAV9-1 | TRAJ12 | m | 14 |
| 80 | TRBV13-3 | TRBJ2-2 | TRAV6-5 | TRAJ23 | m | 16 |
| 81 | NA | NA | NA | NA | m | 18 |
| 82 | TRBV13-2 | TRBJ1-5 | TRAV6-5 | TRAJ27 | m | 20 |
| 83 | TRBV12-1 | TRBJ1-1 | TRAV6N-5 | TRAJ12 | m | 22 |
| 84 | TRBV12-1 | TRBJ1-1 | TRAV6N-5 | TRAJ12 | m | 24 |
| 85 | TRBV13-2 | TRBJ2-3 | NA | NA | o | 2 |
| 86 | TRBV2 | TRBJ2-5 | TRAV6-5 | TRAJ23 | o | 4 |
| 87 | TRBV12-1 | TRBJ1-1 | TRAV6-6 | TRAJ12 | o | 6 |
| 88 | TRBV13-3 | TRBJ2-2 | TRAV6-5 | TRAJ23 | o | 8 |
| 89 | TRBV13-3 | TRBJ2-2 | TRAV6-5 | TRAJ23 | o | 10 |
| 90 | NA | NA | NA | NA | o | 12 |
| 91 | TRBV13-3 | TRBJ2-2 | TRAV6-5 | TRAJ23 | o | 14 |
| 92 | TRBV12-1 | TRBJ1-1 | TRAV6-6 | TRAJ12 | o | 16 |
| 93 | TRBV12-1 | TRBJ1-1 | TRAV6N-5 | TRAJ12 | o | 18 |
| 94 | TRBV13-3 | TRBJ2-2 | TRAV6-5 | TRAJ23 | o | 20 |
| 95 | TRBV13-1 | TRBJ2-5 | TRAV14-1 | TRAJ23 | o | 22 |
| 96 | TRBV13-1 | TRBJ2-5 | TRAV12-2 | TRAJ21 | o | 24 |
| 1 | TRBV13-1 | TRBJ1-5 | TRAV7-4 | TRAJ40 | b | 1 |
| 2 | TRBV14 | TRBJ2-7 | TRAV6-7-DV9 | TRAJ49 | b | 3 |
| 3 | TRBV16 | TRBJ2-4 | TRAV16D-DV11 | TRAJ26 | b | 5 |
| 4 | TRBV13-3 | TRBJ2-2 | TRAV6-5 | TRAJ23 | b | 7 |
| 5 | TRBV29 | TRBJ1-5 | TRAV6-5 | TRAJ44 | b | 9 |
| 6 | TRBV13-2 | TRBJ1-5 | TRAV6-5 | TRAJ27 | b | 11 |
| 7 | TRBV13-3 | TRBJ2-2 | TRAV6-5 | TRAJ23 | b | 13 |
| 8 | TRBV13-2 | TRBJ1-5 | TRAV6-5 | TRAJ27 | b | 15 |
| 9 | TRBV31 | TRBJ2-7 | TRAV7-1 | TRAJ32 | b | 17 |
| 10 | TRBV12-1 | TRBJ2-2 | TRAV9D-3 | TRAJ2 | b | 19 |
| 11 | TRBV12-1 | TRBJ1-1 | TRAV6N-5 | TRAJ12 | b | 21 |
| 12 | TRBV12-1 | TRBJ2-2 | TRAV9D-3 | TRAJ2 | b | 23 |
| 13 | TRBV2 | TRBJ2-5 | TRAV6-5 | TRAJ23 | d | 1 |
| 14 | TRBV13-3 | TRBJ2-2 | TRAV6-5 | TRAJ23 | d | 3 |
| 15 | TRBV12-1 | TRBJ1-1 | TRAV6N-5 | TRAJ12 | d | 5 |
| 16 | TRBV12-1 | TRBJ1-1 | TRAV6N-5 | TRAJ12 | d | 7 |
| 17 | TRBV31 | TRBJ2-1 | TRAV12-1 | TRAJ27 | d | 9 |
| 18 | TRBV13-3 | TRBJ2-2 | TRAV6-5 | TRAJ23 | d | 11 |
| 19 | TRBV13-1 | TRBJ1-5 | TRAV7-4 | TRAJ40 | d | 13 |
| 20 | TRBV16 | TRBJ2-4 | TRAV16D-DV11 | TRAJ26 | d | 15 |
| 21 | TRBV13-3 | TRBJ2-2 | TRAV6-5 | TRAJ23 | d | 17 |
| 22 | TRBV13-2 | TRBJ2-3 | TRAV10 | TRAJ22 | d | 19 |
| 23 | TRBV12-1 | TRBJ1-1 | TRAV6N-5 | TRAJ12 | d | 21 |
| 24 | TRBV12-1 | TRBJ1-1 | TRAV6N-5 | TRAJ12 | d | 23 |
| 25 | TRBV16 | TRBJ2-5 | TRAV16N | TRAJ37 | f | 1 |
| 26 | TRBV13-2 | TRBJ2-3 | TRAV10 | TRAJ22 | f | 3 |
| 27 | TRBV13-2 | TRBJ2-3 | TRAV10 | TRAJ22 | f | 5 |
| 28 | TRBV12-1 | TRBJ2-2 | TRAV9D-3 | TRAJ2 | f | 7 |
| 29 | TRBV13-2 | TRBJ1-4 | NA | NA | f | 9 |
| 30 | TRBV2 | TRBJ2-5 | TRAV6-5 | TRAJ23 | f | 11 |
| 31 | TRBV13-1 | TRBJ1-5 | TRAV7-4 | TRAJ40 | f | 13 |
| 32 | TRBV31 | TRBJ2-7 | TRAV7-1 | TRAJ32 | f | 15 |
| 33 | TRBV13-3 | TRBJ2-2 | TRAV6-5 | TRAJ23 | f | 17 |
| 34 | TRBV2 | TRBJ2-5 | TRAV6-5 | TRAJ23 | f | 19 |
| 35 | TRBV13-3 | TRBJ2-2 | TRAV6-5 | TRAJ23 | f | 21 |
| 36 | TRBV13-1 | TRBJ2-5 | TRAV12-2 | TRAJ21 | f | 23 |
| 37 | NA | NA | NA | NA | h | 1 |
| 38 | TRBV12-1 | TRBJ2-2 | TRAV9D-3 | TRAJ2 | h | 3 |
| 39 | TRBV13-3 | TRBJ2-2 | TRAV6-5 | TRAJ23 | h | 5 |
| 40 | TRBV13-3 | TRBJ2-5 | TRAV10D | TRAJ18 | h | 7 |
| 41 | TRBV13-3 | TRBJ2-2 | TRAV6-5 | TRAJ23 | h | 9 |
| 42 | TRBV12-1 | TRBJ1-2 | NA | NA | h | 11 |
| 43 | TRBV14 | TRBJ2-7 | TRAV6-7-DV9 | TRAJ49 | h | 13 |
| 44 | TRBV2 | TRBJ2-5 | TRAV6-5 | TRAJ23 | h | 15 |
| 45 | TRBV12-1 | TRBJ1-1 | TRAV6-6 | TRAJ12 | h | 17 |
| 46 | TRBV16 | TRBJ2-4 | TRAV16D-DV11 | TRAJ26 | h | 19 |
| 47 | TRBV13-1 | TRBJ2-5 | TRAV12-2 | TRAJ21 | h | 21 |
| 48 | TRBV12-1 | TRBJ2-2 | TRAV9D-3 | TRAJ2 | h | 23 |
| 49 | TRBV31 | TRBJ2-1 | TRAV12-1 | TRAJ27 | j | 1 |
| 50 | TRBV13-3 | TRBJ2-2 | TRAV6-5 | TRAJ23 | j | 3 |
| 51 | TRBV16 | TRBJ2-4 | TRAV16D-DV11 | TRAJ26 | j | 5 |
| 52 | TRBV13-1 | TRBJ1-5 | TRAV7-4 | TRAJ40 | j | 7 |
| 53 | TRBV13-2 | TRBJ1-5 | TRAV6-5 | TRAJ27 | j | 9 |
| 54 | TRBV13-3 | TRBJ2-2 | TRAV6-5 | TRAJ23 | j | 11 |
| 55 | TRBV4 | TRBJ1-5 | TRAV6-6 | TRAJ43 | j | 13 |
| 56 | TRBV12-1 | TRBJ1-1 | TRAV6N-5 | TRAJ12 | j | 15 |
| 57 | TRBV13-1 | TRBJ1-5 | TRAV7-4 | TRAJ40 | j | 17 |
| 58 | TRBV12-1 | TRBJ1-1 | TRAV6N-5 | TRAJ12 | j | 19 |
| 59 | TRBV16 | TRBJ2-4 | TRAV16D-DV11 | TRAJ26 | j | 21 |
| 60 | TRBV12-1 | TRBJ1-1 | TRAV6-6 | TRAJ12 | j | 23 |
| 61 | TRBV12-1 | TRBJ1-2 | NA | NA | l | 1 |
| 62 | TRBV13-3 | TRBJ2-2 | TRAV6-5 | TRAJ23 | l | 3 |
| 63 | TRBV13-3 | TRBJ2-2 | TRAV6-5 | TRAJ23 | l | 5 |
| 64 | TRBV13-2 | TRBJ2-3 | TRAV10 | TRAJ22 | l | 7 |
| 65 | TRBV13-3 | TRBJ2-2 | TRAV6-5 | TRAJ23 | l | 9 |
| 66 | TRBV12-1 | TRBJ1-1 | TRAV6-6 | TRAJ12 | l | 11 |
| 67 | TRBV13-3 | TRBJ2-2 | TRAV6-5 | TRAJ23 | l | 13 |
| 68 | TRBV13-1 | TRBJ2-5 | TRAV12-2 | TRAJ21 | l | 15 |
| 69 | TRBV29 | TRBJ1-5 | TRAV6-5 | TRAJ44 | l | 17 |
| 70 | TRBV13-3 | TRBJ2-2 | TRAV6-5 | TRAJ23 | l | 19 |
| 71 | TRBV31 | TRBJ2-1 | TRAV12-1 | TRAJ27 | l | 21 |
| 72 | TRBV16 | TRBJ2-4 | TRAV16D-DV11 | TRAJ26 | l | 23 |
| 73 | TRBV12-1 | TRBJ1-2 | TRAV6-1 | TRAJ12 | n | 1 |
| 74 | TRBV13-2 | TRBJ2-3 | TRAV10 | TRAJ22 | n | 3 |
| 75 | TRBV4 | TRBJ1-5 | TRAV6-6 | TRAJ43 | n | 5 |
| 76 | TRBV12-1 | TRBJ1-1 | TRAV6-6 | TRAJ12 | n | 7 |
| 77 | NA | NA | NA | NA | n | 9 |
| 78 | TRBV31 | TRBJ2-7 | TRAV7-1 | TRAJ32 | n | 11 |
| 79 | TRBV16 | TRBJ2-4 | TRAV16D-DV11 | TRAJ26 | n | 13 |
| 80 | TRBV13-3 | TRBJ2-2 | TRAV6-5 | TRAJ23 | n | 15 |
| 81 | TRBV13-1 | TRBJ1-5 | TRAV7-4 | TRAJ40. | n | 17 |
| 82 | TRBV13-3 | TRBJ1-1 | TRAV12-2 | TRAJ44 | n | 19 |
| 83 | TRBV2 | TRBJ2-5 | TRAV6-5 | TRAJ23 | n | 21 |
| 84 | TRBV16 | TRBJ2-4 | TRAV16D-DV11 | TRAJ26 | n | 23 |
| 85 | TRBV31 | TRBJ2-1 | NA | NA | p | 1 |
| 86 | TRBV13-3 | TRBJ2-2 | TRAV6-5 | TRAJ23 | p | 3 |
| 87 | TRBV16 | TRBJ2-4 | TRAV16D-DV11 | TRAJ26 | p | 5 |
| 88 | TRBV16 | TRBJ2-4 | TRAV16D-DV11 | TRAJ26 | p | 7 |
| 89 | TRBV13-1 | TRBJ1-5 | TRAV7-4 | TRAJ40 | p | 9 |
| 90 | TRBV13-1 | TRBJ1-5 | TRAV7-4 | TRAJ40 | p | 11 |
| 91 | TRBV4 | TRBJ1-5 | TRAV6-6 | TRAJ43 | p | 13 |
| 92 | TRBV12-1 | TRBJ1-1 | TRAV6-6 | TRAJ12 | p | 15 |
| 93 | TRBV13-2 | TRBJ1-5 | TRAV6-5 | TRAJ27 | p | 17 |
| 94 | TRBV31 | TRBJ2-1 | NA | NA | p | 19 |
| 95 | TRBV31 | TRBJ2-1 | NA | NA | p | 21 |
| 96 | NA | NA | NA | NA | p | 23 |
| 1 | TRBV13-3 | TRBJ2-2 | TRAV6-5 | TRAJ23 | b | 2 |
| 2 | TRBV31 | TRBJ2-1 | TRAV12-1 | TRAJ27 | b | 4 |
| 3 | TRBV12-1 | TRBJ1-2 | TRAV6-1 | TRAJ12 | b | 6 |
| 4 | TRBV13-1 | TRBJ1-4 | NA | NA | b | 8 |
| 5 | TRBV16 | TRBJ2-4 | TRAV16D-DV11 | TRAJ26 | b | 10 |
| 6 | NA | NA | NA | NA | b | 12 |
| 7 | TRBV12-1 | TRBJ1-2 | TRAV6-1 | TRAJ12 | b | 14 |
| 8 | TRBV16 | TRBJ2-4 | TRAV16D-DV11 | TRAJ26 | b | 16 |
| 9 | TRBV13-3 | TRBJ2-2 | TRAV6-5 | TRAJ23 | b | 18 |
| 10 | TRBV13-3 | TRBJ2-2 | TRAV6-5 | TRAJ23 | b | 20 |
| 11 | TRBV12-1 | TRBJ2-2 | TRAV9D-3 | TRAJ2 | b | 22 |
| 12 | TRBV12-1 | TRBJ1-1 | TRAV6N-5 | TRAJ12 | b | 24 |
| 13 | TRBV13-1 | TRBJ1-5 | TRAV7-4 | TRAJ40 | d | 2 |
| 14 | TRBV13-2 | TRBJ2-1 | TRAV6-3 | TRAJ33 | d | 4 |
| 15 | TRBV13-3 | TRBJ2-2 | TRAV6-5 | TRAJ23 | d | 6 |
| 16 | TRBV12-1 | TRBJ2-2 | TRAV9D-3 | TRAJ2 | d | 8 |
| 17 | TRBV16 | TRBJ2-4 | TRAV16D-DV11 | TRAJ26 | d | 10 |
| 18 | NA | NA | NA | NA | d | 12 |
| 19 | TRBV13-3 | TRBJ2-2 | TRAV6-5 | TRAJ23 | d | 14 |
| 20 | TRBV12-1 | TRBJ2-2 | TRAV9D-3 | TRAJ2 | d | 16 |
| 21 | TRBV16 | TRBJ2-4 | TRAV16D-DV11 | TRAJ26 | d | 18 |
| 22 | TRBV12-1 | TRBJ1-2 | TRAV6-1 | TRAJ12 | d | 20 |
| 23 | TRBV12-1 | TRBJ1-1 | TRAV6N-5 | TRAJ12 | d | 22 |
| 24 | TRBV13-2 | TRBJ2-3 | TRAV10 | TRAJ22 | d | 24 |
| 25 | TRBV13-3 | TRBJ2-2 | NA | NA | f | 2 |
| 26 | TRBV13-1 | TRBJ2-7 | NA | NA | f | 4 |
| 27 | TRBV12-1 | TRBJ1-1 | TRAV6N-5 | TRAJ12 | f | 6 |
| 28 | TRBV13-3 | TRBJ2-2 | TRAV6-5 | TRAJ23 | f | 8 |
| 29 | TRBV13-1 | TRBJ2-7 | NA | NA | f | 10 |
| 30 | NA | NA | NA | NA | f | 12 |
| 31 | TRBV12-1 | TRBJ1-1 | TRAV6N-5 | TRAJ12 | f | 14 |
| 32 | TRBV31 | TRBJ2-1 | TRAV12-1 | TRAJ27 | f | 16 |
| 33 | TRBV13-1 | TRBJ1-5 | TRAV7-4 | TRAJ40 | f | 18 |
| 34 | TRBV16 | TRBJ2-4 | TRAV16D-DV11 | TRAJ26 | f | 20 |
| 35 | TRBV12-1 | TRBJ1-1 | TRAV6-6 | TRAJ12 | f | 22 |
| 36 | TRBV31 | TRBJ2-1 | NA | NA | f | 24 |
| 37 | TRBV12-1 | TRBJ1-2 | NA | NA | h | 2 |
| 38 | TRBV12-1 | TRBJ2-2 | TRAV9D-3 | TRAJ2 | h | 4 |
| 39 | TRBV12-1 | TRBJ1-1 | TRAV6N-5 | TRAJ12 | h | 6 |
| 40 | TRBV31 | TRBJ2-1 | TRAV12-1 | TRAJ27 | h | 8 |
| 41 | TRBV13-2 | TRBJ2-3 | TRAV10 | TRAJ22 | h | 10 |
| 42 | NA | NA | NA | NA | h | 12 |
| 43 | TRBV14 | TRBJ2-7 | TRAV6-7-DV9 | TRAJ49 | h | 14 |
| 44 | TRBV13-1 | TRBJ2-7 | TRAV14D-3-DV8 | TRAJ11 | h | 16 |
| 45 | TRBV13-2 | TRBJ2-3 | TRAV10 | TRAJ22 | h | 18 |
| 46 | TRBV12-1 | TRBJ1-2 | TRAV6-6 | TRAJ12 | h | 20 |
| 47 | TRBV31 | TRBJ2-1 | NA | NA | h | 22 |
| 48 | TRBV12-1 | TRBJ1-1 | TRAV6N-5 | TRAJ12 | h | 24 |
| 49 | TRBV29 | TRBJ1-5 | TRAV6-5 | TRAJ44 | j | 2 |
| 50 | TRBV16 | TRBJ2-4 | TRAV16D-DV11 | TRAJ26 | j | 4 |
| 51 | TRBV13-1 | TRBJ2-5 | TRAV12-2 | TRAJ21 | j | 6 |
| 52 | TRBV13-1 | TRBJ2-5 | TRAV12-2 | TRAJ21 | j | 8 |
| 53 | NA | NA | NA | NA | j | 10 |
| 54 | NA | NA | NA | NA | j | 12 |
| 55 | TRBV13-1 | TRBJ2-5 | TRAV12-2 | TRAJ21 | j | 14 |
| 56 | TRBV13-2 | TRBJ1-5 | TRAV6-5 | TRAJ27 | j | 16 |
| 57 | TRBV31 | TRBJ2-1 | TRAV3-1 | TRAJ24 | j | 18 |
| 58 | TRBV2 | TRBJ2-5 | TRAV6-5 | TRAJ23 | j | 20 |
| 59 | TRBV12-1 | TRBJ1-1 | TRAV6N-5 | TRAJ12 | j | 22 |
| 60 | TRBV31 | TRBJ2-7 | TRAV7-1 | TRAJ32 | j | 24 |
| 61 | TRBV31 | TRBJ2-1 | TRAV13-2 | TRAJ50 | l | 2 |
| 62 | TRBV2 | TRBJ2-5 | TRAV6-5 | TRAJ23 | l | 4 |
| 63 | TRBV12-1 | TRBJ1-1 | TRAV6N-5 | TRAJ12 | l | 6 |
| 64 | TRBV13-2 | TRBJ2-3 | TRAV10 | TRAJ22 | l | 8 |
| 65 | TRBV12-1 | TRBJ1-1 | TRAV6N-5 | TRAJ12 | l | 10 |
| 66 | NA | NA | NA | NA | l | 12 |
| 67 | TRBV16 | TRBJ2-4 | TRAV16D-DV11 | TRAJ26 | l | 14 |
| 68 | TRBV12-1 | TRBJ1-2 | NA | NA | l | 16 |
| 69 | TRBV12-1 | TRBJ1-2 | NA | NA | l | 18 |
| 70 | TRBV13-3 | TRBJ2-2 | TRAV6-5 | TRAJ23 | l | 20 |
| 71 | TRBV12-1 | TRBJ1-2 | TRAV6-1 | TRAJ12 | l | 22 |
| 72 | TRBV12-1 | TRBJ1-2 | TRAV6N-5 | TRAJ12 | l | 24 |
| 73 | TRBV2 | TRBJ2-5 | TRAV6-5 | TRAJ23 | n | 2 |
| 74 | TRBV12-1 | TRBJ1-2 | TRAV6N-5 | TRAJ12 | n | 4 |
| 75 | TRBV13-3 | TRBJ2-2 | TRAV6-5 | TRAJ23 | n | 6 |
| 76 | NA | NA | NA | NA | n | 8 |
| 77 | TRBV13-2 | TRBJ2-1 | TRAV6-3 | TRAJ33 | n | 10 |
| 78 | NA | NA | NA | NA | n | 12 |
| 79 | TRBV31 | TRBJ2-1 | TRAV12-1 | TRAJ27 | n | 14 |
| 80 | TRBV12-1 | TRBJ2-2 | TRAV9D-3 | TRAJ2 | n | 16 |
| 81 | TRBV13-3 | TRBJ2-2 | TRAV6-5 | TRAJ23 | n | 18 |
| 82 | TRBV13-1 | TRBJ1-5 | TRAV7-4 | TRAJ40 | n | 20 |
| 83 | TRBV13-2 | TRBJ2-3 | TRAV10 | TRAJ22 | n | 22 |
| 84 | TRBV13-3 | TRBJ2-2 | TRAV6-5 | TRAJ23 | n | 24 |
| 85 | TRBV12-1 | TRBJ1-1 | TRAV6-6 | TRAJ12 | p | 2 |
| 86 | TRBV31 | TRBJ2-1 | TRAV14D-1 | TRAJ33 | p | 4 |
| 87 | TRBV13-1 | TRBJ2-5 | TRAV12-2 | TRAJ21 | p | 6 |
| 88 | TRBV13-1 | TRBJ2-5 | NA | NA | p | 8 |
| 89 | TRBV12-1 | TRBJ1-2 | NA | NA | p | 10 |
| 90 | NA | NA | NA | NA | p | 12 |
| 91 | TRBV2 | TRBJ2-5 | TRAV6-5 | TRAJ23 | p | 14 |
| 92 | TRBV12-1 | TRBJ1-2 | TRAV6-1 | TRAJ12 | p | 16 |
| 93 | TRBV13-2 | TRBJ2-3 | TRAV10 | TRAJ22 | p | 18 |
| 94 | TRBV13-3 | TRBJ2-2 | TRAV6-5 | TRAJ23 | p | 20 |
| 95 | TRBV12-1 | TRBJ1-2 | TRAV6-1 | TRAJ12 | p | 22 |

NA, not available.

What is claimed is:

1. A kit for analyzing a T cell receptor of a single T cell comprising:
(a) a first set of primers comprising a collection of first forward primers and a first reverse primer for each chain of the T cell receptor, said first set of primers amplifying a nucleic acid molecule encoding a portion of the T cell receptor comprising the hypervariable CDR3 region, wherein each first reverse primer hybridizes to a sequence encoding the constant segment of the T cell receptor chain; and
(b) a second set of primers comprising a collection of second forward primers and a collection of second reverse primers for each chain of the T cell receptor, said second set of primers amplifying a portion of the nucleic acid molecule of (a) comprising the hypervariable CDR3 region, wherein each of the second reverse primers comprises:
(i) a sequence that hybridizes the constant segment of the T cell receptor chain,
(ii) a unique barcode, and
(iii) a sequence identifying the chain of the T cell receptor.

2. The kit of claim 1, wherein the T cell receptor comprises a α and β chain or a γ and δ chain.

3. The kit of claim 1, wherein the collection of first forward primers comprises the nucleotide sequences of:
(i) SEQ ID NOs:1-40 and SEQ ID NOs:42-70;
(ii) SEQ ID NOs:72-80 and SEQ ID NOs:82-89;
(iii) SEQ ID NOs:181-203 and SEQ ID NOs:205-223; or
(iv) SEQ ID NOs:225-229 and SEQ ID NOs:231-243.

4. The kit of claim 1, wherein the first reverse primer for each chain of the T cell receptor comprises the nucleotide sequences of:
(i) SEQ ID NO:41 and SEQ ID NO:71;
(ii) SEQ ID NO:81 and SEQ ID NO:90;
(iii) SEQ ID NO:204 and SEQ ID NO:224; or
(iv) SEQ ID NO:230 and SEQ ID NO: 244;

5. The kit of claim 1, wherein the collection of second forward primers comprises the nucleotide sequences of:
(i) SEQ ID NOS:91-130 and SEQ ID NOs:132-160;
(ii) SEQ ID NOs:162-170 and SEQ ID NOs:172-179;
(iii) SEQ ID NOs:245-267 and SEQ ID NOs:269-287; or (iv) SEQ ID NOs:289-293 and SEQ ID NOs:295-307.

6. The kit of claim 1, wherein the collection of second reverse primers comprises the sequences:
(i) CGACTCAAGTGTGTGGXXXXXXGGGTCAGGGTTCTGGATAT (SEQ ID NO:744) and CGACTCAGATTGGTACXXXXXXACACSTTKTTCAGGTCCTC (SEQ ID NO:745); or
(ii) CGACTCAAGTGTGTGGXXXXXXTTCTGGGTTCTGGATGT (SEQ ID NO:746) and CGACTCAGATTGGTACXXXXXXAGTCACATTTCTCAGATCCT (SEQ ID NO:747),
wherein XXXXXX is a unique barcode.

7. The kit of claim 6, wherein the collection of second reverse primers comprises the sequences:
(i) SEQ ID NOS:360-455 and SEQ ID NOs:456-551; or
(ii) SEQ ID NOs:552-647 and SEQ ID NOs:648-743.

8. The kit of claim 1, further comprising Cellular Indexing of the Transcriptome and Epitopes by Sequencing (CITE-Seq) primers.

9. The kit of claim 8, wherein the CITE-Seq primers comprise the sequences:
(i) SEQ ID NO:356 and SEQ ID NO:357; or
(ii) SEQ ID NO:358 and SEQ ID NO:359.

10. A method for analyzing a T cell receptor of a single T cell comprising:
(a) sorting single T cells from a sample comprising a plurality of T cells into separate locations;
(b) amplifying nucleic acid molecules encoding chains of the T cell receptor from one or more single T cells using the first set of primers from the kit of claim 1 to produce a first set of amplicon products in one or more locations of the separate locations;
(c) performing nested polymerase chain reaction (PCR) on the amplified nucleic acid molecules encoding the chains of the T cell receptor in the first set of amplicon products with the second set of primers from the kit of claim 1 to produce a second set of amplicon products; and
(d) sequencing the amplicon products.

11. The method of claim 10, wherein the T cell receptor comprises a α and β chain or a γ and δ chain.

12. The method of claim 10, wherein the sample is collected from a subject.

13. The method of claim 10, wherein the step of (d) sequencing the amplicon products comprises ligating sequencing adapters onto the second set of amplicon products and sequencing the amplicon products by next generation sequencing.

14. The method of claim 10, further comprising introducing Cellular Indexing of the Transcriptome and Epitopes by Sequencing primers into the amplifying step of (b).


SEQUENCE LISTING (755 sequences)

SEQ ID NO: 1 - DNA, 20 bp, synthetic construct
  source 1..20: mol_type = other DNA
aactgcacgtaccagacatc

SEQ ID NO: 2 - DNA, 18 bp, synthetic construct
  source 1..18: mol_type = other DNA
gatgtgcaccaagactcc

SEQ ID NO: 3 - DNA, 19 bp, synthetic construct
  source 1..19: mol_type = other DNA
aagatcaggtcaacgttgc

SEQ ID NO: 4 - DNA, 21 bp, synthetic construct
  source 1..21: mol_type = other DNA
ctccatggactcatatgaagg

SEQ ID NO: 5 - DNA, 19 bp, synthetic construct
  source 1..19: mol_type = other DNA
cttttcctgagtgtccgag

SEQ ID NO: 6 - DNA, 21 bp, synthetic construct
  source 1..21: mol_type = other DNA
caccctgacctgcaactatac

SEQ ID NO: 7 - DNA, 18 bp, synthetic construct
  source 1..18: mol_type = other DNA
gcaaaatacagggatggg

SEQ ID NO: 8 - DNA, 18 bp, synthetic construct
  source 1..18: mol_type = other DNA
ctcactggagttgggatg

SEQ ID NO: 9 - DNA, 19 bp, synthetic construct
  source 1..19: mol_type = other DNA
cactgtctctgaaggagcc

SEQ ID NO: 10 - DNA, 17 bp, synthetic construct
  source 1..17: mol_type = other DNA
gccaccctggttaaagg

SEQ ID NO: 11 - DNA, 20 bp, synthetic construct
  source 1..20: mol_type = other DNA
gagctgaggtgcaactactc

SEQ ID NO: 12 - DNA, 18 bp, synthetic construct
  source 1..18: mol_type = other DNA
ctaacagaggccacccag

SEQ ID NO: 13 - DNA, 20 bp, synthetic construct
  source 1..20: mol_type = other DNA
tggtatgtccaatatcctgg

SEQ ID NO: 14 - DNA, 19 bp, synthetic construct
  source 1..19: mol_type = other DNA
caagtggagcagagtcctc

SEQ ID NO: 15 - DNA, 18 bp, synthetic construct
  source 1..18: mol_type = other DNA
cartgttccagagggagc

SEQ ID NO: 16 - DNA, 19 bp, synthetic construct
  source 1..19: mol_type = other DNA
catccttcaaccctgagtg

SEQ ID NO: 17 - DNA, 19 bp, synthetic construct
  source 1..19: mol_type = other DNA
cagcgcctcagactacttc

SEQ ID NO: 18 - DNA, 22 bp, synthetic construct
  source 1..22: mol_type = other DNA
aagataactcaaacccaaccag

SEQ ID NO: 19 - DNA, 19 bp, synthetic construct
  source 1..19: mol_type = other DNA
agtggagctgaagtgcaac

SEQ ID NO: 20 - DNA, 19 bp, synthetic construct
  source 1..19: mol_type = other DNA
ggagaagaggatcctcagg

SEQ ID NO: 21 - DNA, 22 bp, synthetic construct
  source 1..22: mol_type = other DNA
tccagtatctaaacaaagagcc

SEQ ID NO: 22 - DNA, 19 bp, synthetic construct
  source 1..19: mol_type = other DNA
aggtaactcaagcgcagac

SEQ ID NO: 23 - DNA, 20 bp, synthetic construct
  source 1..20: mol_type = other DNA
cacagtcagcggtttaagag

SEQ ID NO: 24 - DNA, 18 bp, synthetic construct
  source 1..18: mol_type = other DNA
ttcctgcagctctgagtg

SEQ ID NO: 25 - DNA, 20 bp, synthetic construct
  source 1..20: mol_type = other DNA
gtcctccagacctgattctc

SEQ ID NO: 26 - DNA, 19 bp, synthetic construct
  source 1..19: mol_type = other DNA
tgcttatgagaacactgcg

SEQ ID NO: 27 - DNA, 20 bp, synthetic construct
  source 1..20: mol_type = other DNA
ctcagtcactgcatgttcag

SEQ ID NO: 28 - DNA, 19 bp, synthetic construct
  source 1..19: mol_type = other DNA
ggacttcaccacgtactgc

SEQ ID NO: 29 - DNA, 19 bp, synthetic construct
  source 1..19: mol_type = other DNA
gcaaacctgccttgtaatc

SEQ ID NO: 30 - DNA, 19 bp, synthetic construct
  source 1..19: mol_type = other DNA
agccaaattcaatggagag

SEQ ID NO: 31 - DNA, 22 bp, synthetic construct
  source 1..22: mol_type = other DNA
tcagtttctaagcatccaagag

SEQ ID NO: 32 - DNA, 21 bp, synthetic construct
  source 1..21: mol_type = other DNA
gcaagttaagcaaaattcacc

SEQ ID NO: 33 - DNA, 19 bp, synthetic construct
  source 1..19: mol_type = other DNA
caacaaccagtgcagagtc

SEQ ID NO: 34 - DNA, 20 bp, synthetic construct
  source 1..20: mol_type = other DNA
agaactggagcagagtcctc

SEQ ID NO: 35 - DNA, 20 bp, synthetic construct
  source 1..20: mol_type = other DNA
ggtcaacagctgaatcagag

SEQ ID NO: 36 - DNA, 21 bp, synthetic construct
  source 1..21: mol_type = other DNA
gaagacaaggtggtacaaagc

SEQ ID NO: 37 - DNA, 20 bp, synthetic construct
  source 1..20: mol_type = other DNA
gcacatatgacaccagtgag

SEQ ID NO: 38 - DNA, 18 bp, synthetic construct
  source 1..18: mol_type = other DNA
ctgttcctgagcatgcag

SEQ ID NO: 39 - DNA, 21 bp, synthetic construct
  source 1..21: mol_type = other DNA
gcatctgtgactatgaactgc

SEQ ID NO: 40 - DNA, 20 bp, synthetic construct
  source 1..20: mol_type = other DNA
aatgaagtggagcagagtcc

SEQ ID NO: 41 - DNA, 19 bp, synthetic construct
  source 1..19: mol_type = other DNA
gaccagcttgacatcacag

SEQ ID NO: 42 - DNA, 21 bp, synthetic construct
  source 1..21: mol_type = other DNA
tcgatgatcaattctcagttg

SEQ ID NO: 43 - DNA, 20 bp, synthetic construct
  source 1..20: mol_type = other DNA
caaaatacctggtcacacag

SEQ ID NO: 44 - DNA, 18 bp, synthetic construct
  source 1..18: mol_type = other DNA
tcgcttctcacctgaatg

SEQ ID NO: 45 - DNA, 20 bp, synthetic construct
  source 1..20: mol_type = other DNA
gattctcaggkckccagttc

SEQ ID NO: 46 - DNA, 19 bp, synthetic construct
  source 1..19: mol_type = other DNA
gtaccaacaggycctgggt

SEQ ID NO: 47 - DNA, 19 bp, synthetic construct
  source 1..19: mol_type = other DNA
actcagaccccaaaattcc

SEQ ID NO: 48 - DNA, 19 bp, synthetic construct
  source 1..19: mol_type = other DNA
actggcaaaggagaagtcc

SEQ ID NO: 49 - DNA, 20 bp, synthetic construct
  source 1..20: mol_type = other DNA
trtgatccaatttcaggtca

SEQ ID NO: 50 - DNA, 19 bp, synthetic construct
  source 1..19: mol_type = other DNA
cgswtctytgcagaraggc

SEQ ID NO: 51 - DNA, 19 bp, synthetic construct
  source 1..19: mol_type = other DNA
gatcacagcaactggacag

SEQ ID NO: 52 - DNA, 18 bp, synthetic construct
  source 1..18: mol_type = other DNA
cagagcccaagacacaag

SEQ ID NO: 53 - DNA, 20 bp, synthetic construct
  source 1..20: mol_type = other DNA
accttgatgtgtcaccagac

SEQ ID NO: 54 - DNA, 18 bp, synthetic construct
  source 1..18: mol_type = other DNA
cagagcccaagacacaag

SEQ ID NO: 55 - DNA, 19 bp, synthetic construct
  source 1..19: mol_type = other DNA
cgattttctgcagagacgc

SEQ ID NO: 56 - DNA, 20 bp, synthetic construct
  source 1..20: mol_type = other DNA
argtgacagaratgggacaa

SEQ ID NO: 57 - DNA, 19 bp, synthetic construct
  source 1..19: mol_type = other DNA
agcgataaaggaagcatcc

SEQ ID NO: 58 - DNA, 21 bp, synthetic construct
  source 1..21: mol_type = other DNA
ccaacaatcgattcttagctg

SEQ ID NO: 59 - DNA, 20 bp, synthetic construct
  source 1..20: mol_type = other DNA
agtgaccctgagttgttctc

SEQ ID NO: 60 - DNA, 22 bp, synthetic construct
  source 1..22: mol_type = other DNA
gtctttgatgaaacaggtatgc

SEQ ID NO: 61 - DNA, 18 bp, synthetic construct
  source 1..18: mol_type = other DNA
cagacccccagacacaag

SEQ ID NO: 62 - DNA, 21 bp, synthetic construct
  source 1..21: mol_type = other DNA
catagatgagtcaggaatgcc

SEQ ID NO: 63 - DNA, 22 bp, synthetic construct
  source 1..22: mol_type = other DNA
agttgtgaacagaatttgaacc

SEQ ID NO: 64 - DNA, 20 bp, synthetic construct
  source 1..20: mol_type = other DNA
aagtttctcatcaaccatgc

SEQ ID NO: 65 - DNA, 19 bp, synthetic construct
  source 1..19: mol_type = other DNA
gcgattctcatctcaatgc

SEQ ID NO: 66 - DNA, 22 bp, synthetic construct
  source 1..22: mol_type = other DNA
cctacggttgatctattactcc

SEQ ID NO: 67 - DNA, 22 bp, synthetic construct
  source 1..22: mol_type = other DNA
actacacctcatccactattcc

SEQ ID NO: 68 - DNA, 19 bp, synthetic construct
  source 1..19: mol_type = other DNA
tggtatcgacaagacccag

SEQ ID NO: 69 - DNA, 19 bp, synthetic construct
  source 1..19: mol_type = other DNA
ttctggtaccgtcagcaac

SEQ ID NO: 70 - DNA, 20 bp, synthetic construct
  source 1..20: mol_type = other DNA
tccagctgctcttctactcc

SEQ ID NO: 71 - DNA, 20 bp, synthetic construct
  source 1..20: mol_type = other DNA
tagaactggacttgacagcg

SEQ ID NO: 72 - DNA, 18 bp, synthetic construct
  source 1..18: mol_type = other DNA
tcttccaacttggaaggg

SEQ ID NO: 73 - DNA, 18 bp, synthetic construct
  source 1..18: mol_type = other DNA
tcttccaacttgcaaggg

SEQ ID NO: 74 - DNA, 18 bp, synthetic construct
  source 1..18: mol_type = other DNA
gggtcatcctgtttccag

SEQ ID NO: 75 - DNA, 18 bp, synthetic construct
  source 1..18: mol_type = other DNA
tggcctcccaaagtactg

SEQ ID NO: 76 - DNA, 19 bp, synthetic construct
  source 1..19: mol_type = other DNA
ccaacttggaagggagaac

SEQ ID NO: 77 - DNA, 19 bp, synthetic construct
  source 1..19: mol_type = other DNA
ccaggtcacctagagcaac

SEQ ID NO: 78 - DNA, 21 bp, synthetic construct
  source 1..21: mol_type = other DNA
ttatcaaaagtggagcagttc

SEQ ID NO: 79 - DNA, 24 bp, synthetic construct
  source 1..24: mol_type = other DNA
gaacaacctgaaatatctatttcc

SEQ ID NO: 80 - DNA, 20 bp, synthetic construct
  source 1..20: mol_type = other DNA
gggtcatctgctgaaatcac

SEQ ID NO: 81 - DNA, 16 bp, synthetic construct
  source 1..16: mol_type = other DNA
ggtgttcccctcctgg

SEQ ID NO: 82 - DNA, 19 bp, synthetic construct
  source 1..19: mol_type = other DNA
gcccagaaggttactcaag

SEQ ID NO: 83 - DNA, 19 bp, synthetic construct
  source 1..19: mol_type = other DNA
attgagttggtgcctgaac

SEQ ID NO: 84 - DNA, 23 bp, synthetic construct
  source 1..23: mol_type = other DNA
tgtgacaaagtaacccagagttc

SEQ ID NO: 85 - DNA, 18 bp, synthetic construct
  source 1..18: mol_type = other DNA
caaacccaaccaggaatg

SEQ ID NO: 86 - DNA, 21 bp, synthetic construct
  source 1..21: mol_type = other DNA
gcaagttaagcaaaattcacc

SEQ ID NO: 87 - DNA, 20 bp, synthetic construct
  source 1..20: mol_type = other DNA
ttgatagtccagaaaggagg

SEQ ID NO: 88 - DNA, 19 bp, synthetic construct
  source 1..19: mol_type = other DNA
gacaaggtggtacaaagcc

SEQ ID NO: 89 - DNA, 21 bp, synthetic construct
  source 1..21: mol_type = other DNA
cagtcactcagtctcaaccag

SEQ ID NO: 90 - DNA, 24 bp, synthetic construct
  source 1..24: mol_type = other DNA
cttcatatttaccaagcttgacag

SEQ ID NO: 91 - DNA, 21 bp, synthetic construct
  source 1..21: mol_type = other DNA
gcacccacatttctktcttac

SEQ ID NO: 92 - DNA, 21 bp, synthetic construct
  source 1..21: mol_type = other DNA
cactctgtgtccaatgcttac

SEQ ID NO: 93 - DNA, 21 bp, synthetic construct
  source 1..21: mol_type = other DNA
atgcacctattcagtctctgg

SEQ ID NO: 94 - DNA, 23 bp, synthetic construct
  source 1..23: mol_type = other DNA
attatatcacgtggtaccaacag

SEQ ID NO: 95 - DNA, 20 bp, synthetic construct
  source 1..20: mol_type = other DNA
tacacagacagctcctccac

SEQ ID NO: 96 - DNA, 19 bp, synthetic construct
  source 1..19: mol_type = other DNA
tggtaccgacaagatccag

SEQ ID NO: 97 - DNA, 22 bp, synthetic construct
  source 1..22: mol_type = other DNA
tatgagaagcagaaaggaagac

SEQ ID NO: 98 - DNA, 20 bp, synthetic construct
  source 1..20: mol_type = other DNA
gtcaacaccttcagcttctc

SEQ ID NO: 99 - DNA, 21 bp, synthetic construct
  source 1..21: mol_type = other DNA
tttgaggctgaatttaagagg

SEQ ID NO: 100 - DNA, 20 bp, synthetic construct
  source 1..20: mol_type = other DNA
agagtgaaacctccttccac

SEQ ID NO: 101 - DNA, 19 bp, synthetic construct
  source 1..19: mol_type = other DNA
aaccaaggactccagcttc

SEQ ID NO: 102 - DNA, 19 bp, synthetic construct
  source 1..19: mol_type = other DNA
atcagaggttttgaggctg

SEQ ID NO: 103 - DNA, 21 bp, synthetic construct
  source 1..21: mol_type = other DNA
gaaaccacttctttccacttg

SEQ ID NO: 104 - DNA, 22 bp, synthetic construct
  source 1..22: mol_type = other DNA
gaaagaactgcactcttcaatg

SEQ ID NO: 105 - DNA, 21 bp, synthetic construct
  source 1..21: mol_type = other DNA
aagatggaaggtttacagcac

SEQ ID NO: 106 - DNA, 21 bp, synthetic construct
  source 1..21: mol_type = other DNA
tcagacagtgcctcaaactac

SEQ ID NO: 107 - DNA, 21 bp, synthetic construct
  source 1..21: mol_type = other DNA
cagtgaaacatctctctctgc

SEQ ID NO: 108 - DNA, 19 bp, synthetic construct
  source 1..19: mol_type = other DNA
aggctgtgactctggactg

SEQ ID NO: 109 - DNA, 20 bp, synthetic construct
  source 1..20: mol_type = other DNA
gtccagtactccagacaacg

SEQ ID NO: 110 - DNA, 20 bp, synthetic construct
  source 1..20: mol_type = other DNA
ccaccatgaactgcagttac

SEQ ID NO: 111 - DNA, 19 bp, synthetic construct
  source 1..19: mol_type = other DNA
tgacagttccttccacctg

SEQ ID NO: 112 - DNA, 19 bp, synthetic construct
  source 1..19: mol_type = other DNA
tgtgaccttggactgtgtg

SEQ ID NO: 113 - DNA, 21 bp, synthetic construct
  source 1..21: mol_type = other DNA
tctggtataggcaagatcctg

SEQ ID NO: 114 - DNA, 20 bp, synthetic construct
  source 1..20: mol_type = other DNA
aacttggttctcaactgcag

SEQ ID NO: 115 - DNA, 21 bp, synthetic construct
  source 1..21: mol_type = other DNA
ctgactctgtgaacaatttgc

SEQ ID NO: 116 - DNA, 21 bp, synthetic construct
  source 1..21: mol_type = other DNA
tgcattattgatagccatacg

SEQ ID NO: 117 - DNA, 21 bp, synthetic construct
  source 1..21: mol_type = other DNA
tgccttacactggtacagatg

SEQ ID NO: 118 - DNA, 19 bp, synthetic construct
  source 1..19: mol_type = other DNA
tataagcaaaggcctggtg

SEQ ID NO: 119 - DNA, 18 bp, synthetic construct
  source 1..18: mol_type = other DNA
cgacagattcactcccag

SEQ ID NO: 120 - DNA, 20 bp, synthetic construct
  source 1..20: mol_type = other DNA
ttcacttgccttgtaaccac

SEQ ID NO: 121 - DNA, 21 bp, synthetic construct
  source 1..21: mol_type = other DNA
ctcactgtgtactgcaactcc

SEQ ID NO: 122 - DNA, 20 bp, synthetic construct
  source 1..20: mol_type = other DNA
ctgctgaaggtcctacattc

SEQ ID NO: 123 - DNA, 18 bp, synthetic construct
  source 1..18: mol_type = other DNA
agaagcatggtgaagcac

SEQ ID NO: 124 - DNA, 20 bp, synthetic construct
  source 1..20: mol_type = other DNA
atctcaccataaactgcacg

SEQ ID NO: 125 - DNA, 19 bp, synthetic construct
  source 1..19: mol_type = other DNA
acctggctatggtacaagc

SEQ ID NO: 126 - DNA, 19 bp, synthetic construct
  source 1..19: mol_type = other DNA
atctctggttgtccacgag

SEQ ID NO: 127 - DNA, 19 bp, synthetic construct
  source 1..19: mol_type = other DNA
cagcaggcagatgattctc

SEQ ID NO: 128 - DNA, 21 bp, synthetic construct
  source 1..21: mol_type = other DNA
tcaaccacttcagacagactg

SEQ ID NO: 129 - DNA, 21 bp, synthetic construct
  source 1..21: mol_type = other DNA
ggaggcggaaatattaaagac

SEQ ID NO: 130 - DNA, 20 bp, synthetic construct
  source 1..20: mol_type = other DNA
ttgtttatgctgagctcagg

SEQ ID NO: 131 - DNA, 21 bp, synthetic construct
  source 1..21: mol_type = other DNA
tgttgctcttgaagtccatag

SEQ ID NO: 132 - DNA, 20 bp, synthetic construct
  source 1..20: mol_type = other DNA
ttcactctgaagatccggtc

SEQ ID NO: 133 - DNA, 21 bp, synthetic construct
  source 1..21: mol_type = other DNA
aatcttcacatcaattccctg

SEQ ID NO: 134 - DNA, 18 bp, synthetic construct
  source 1..18: mol_type = other DNA
cctgcagccagaagactc

SEQ ID NO: 135 - DNA, 18 bp, synthetic construct
  source 1..18: mol_type = other DNA
cttggagctggrsgactc

SEQ ID NO: 136 - DNA, 19 bp, synthetic construct
  source 1..19: mol_type = other DNA
tctgagctgaatgtgaacg

SEQ ID NO: 137 - DNA, 20 bp, synthetic construct
  source 1..20: mol_type = other DNA
gtgtrcccaggatatgaacc

SEQ ID NO: 138 - DNA, 21 bp, synthetic construct
  source 1..21: mol_type = other DNA
tggttatagtgtctccagagc

SEQ ID NO: 139 - DNA, 20 bp, synthetic construct
  source 1..20: mol_type = other DNA
tcyactctgamgwtccagcg

SEQ ID NO: 140 - DNA, 17 bp, synthetic construct
  source 1..17: mol_type = other DNA
tgrmgatycagcgcaca

SEQ ID NO: 141 - DNA, 19 bp, synthetic construct
  source 1..19: mol_type = other DNA
gtaccaacagagcctggac

SEQ ID NO: 142 - DNA, 19 bp, synthetic construct
  source 1..19: mol_type = other DNA
tggtatcgacaagacctgg

SEQ ID NO: 143 - DNA, 19 bp, synthetic construct
  source 1..19: mol_type = other DNA
tggtatcgacaagacctgg

SEQ ID NO: 144 - DNA, 20 bp, synthetic construct
  source 1..20: mol_type = other DNA
ggaacaccagtgactctgag

SEQ ID NO: 145 - DNA, 20 bp, synthetic construct
  source 1..20: mol_type = other DNA
gactccactctcaagatcca

SEQ ID NO: 146 - DNA, 19 bp, synthetic construct
  source 1..19: mol_type = other DNA
cyactctgargatccagcc

SEQ ID NO: 147 - DNA, 21 bp, synthetic construct
  source 1..21: mol_type = other DNA
cattctgaactgaacatgagc

SEQ ID NO: 148 - DNA, 21 bp, synthetic construct
  source 1..21: mol_type = other DNA
attctactctgaaggtgcagc

SEQ ID NO: 149 - DNA, 20 bp, synthetic construct
  source 1..20: mol_type = other DNA
ataacttccaatccaggagg

SEQ ID NO: 150 - DNA, 20 bp, synthetic construct
  source 1..20: mol_type = other DNA
ctgtagccttgagatccagg

SEQ ID NO: 151 - DNA, 19 bp, synthetic construct
  source 1..19: mol_type = other DNA
tgttcactggtaccgacag

SEQ ID NO: 152 - DNA, 20 bp, synthetic construct
  source 1..20: mol_type = other DNA
cgattttctgctgaatttcc

SEQ ID NO: 153 - DNA, 20 bp, synthetic construct
  source 1..20: mol_type = other DNA
ttcctctcactgtgacatcg

SEQ ID NO: 154 - DNA, 20 bp, synthetic construct
  source 1..20: mol_type = other DNA
actctgacagtgaccagtgc

SEQ ID NO: 155 - DNA, 19 bp, synthetic construct
  source 1..19: mol_type = other DNA
gcaatcctgtcctcagaac

SEQ ID NO: 156 - DNA, 21 bp, synthetic construct
  source 1..21: mol_type = other DNA
gatggatacagtgtctctcga

SEQ ID NO: 157 - DNA, 20 bp, synthetic construct
  source 1..20: mol_type = other DNA
cagagaagggagatctttcc

SEQ ID NO: 158 - DNA, 20 bp, synthetic construct
  source 1..20: mol_type = other DNA
ttcyccctgatyctggagtc

SEQ ID NO: 159 - DNA, 21 bp, synthetic construct
  source 1..21: mol_type = other DNA
tctgactgtgagcaacatgag

SEQ ID NO: 160 - DNA, 21 bp, synthetic construct
  source 1..21: mol_type = other DNA
agaatctctcagcctccagac

SEQ ID NO: 161 - DNA, 20 bp, synthetic construct
  source 1..20: mol_type = other DNA
ttctgatggctcaaacacag

SEQ ID NO: 162 - DNA, 19 bp, synthetic construct
  source 1..19: mol_type = other DNA
ggtcatctgctgaaatcac

SEQ ID NO: 163 - DNA, 22 bp, synthetic construct
  source 1..22: mol_type = other DNA
ggtcatctgctgtaatcacttg

SEQ ID NO: 164 - DNA, 22 bp, synthetic construct
  source 1..22: mol_type = other DNA
tacctaaggacctgtgtagagg

SEQ ID NO: 165 - DNA, 20 bp, synthetic construct
  source 1..20: mol_type = other DNA
tcctctttctatgtcccagg

SEQ ID NO: 166 - DNA, 18 bp, synthetic construct
  source 1..18: mol_type = other DNA
aaaatgccgtctacaccc

SEQ ID NO: 167 - DNA, 19 bp, synthetic construct
  source 1..19: mol_type = other DNA
tgtccatttcatatgacgg

SEQ ID NO: 168 - DNA, 19 bp, synthetic construct
  source 1..19: mol_type = other DNA
cagctatccatttccacgg

SEQ ID NO: 169 - DNA, 19 bp, synthetic construct
  source 1..19: mol_type = other DNA
catatcttggaaggcatcc

SEQ ID NO: 170 - DNA, 15 bp, synthetic construct
  source 1..15: mol_type = other DNA
ccaggaggggaaggc

SEQ ID NO: 171 - DNA, 17 bp, synthetic construct
  source 1..17: mol_type = other DNA
cccagaatcgtgttgct

SEQ ID NO: 172 - DNA, 22 bp, synthetic construct
  source 1..22: mol_type = other DNA
agcaaagagatgattttcctta

SEQ ID NO: 173 - DNA, 24 bp, synthetic construct
  source 1..24: mol_type = other DNA
tatatcaactggtacaggaagacc

SEQ ID NO: 174 - DNA, 22 bp, synthetic construct
  source 1..22: mol_type = other DNA
ggtactgctctgcacttacgac

SEQ ID NO: 175 - DNA, 19 bp, synthetic construct
  source 1..19: mol_type = other DNA
aggaaaaggaggctgtgac

SEQ ID NO: 176 - DNA, 20 bp, synthetic construct
  source 1..20: mol_type = other DNA
ctgctgaaggtcctacattc

SEQ ID NO: 177 - DNA, 20 bp, synthetic construct
  source 1..20: mol_type = other DNA
cgtttgactactttccatgg

SEQ ID NO: 178 - DNA, 19 bp, synthetic construct
  source 1..19: mol_type = other DNA
atctctggttgtccacgag

SEQ ID NO: 179 - DNA, 18 bp, synthetic construct
  source 1..18: mol_type = other DNA
tctggtacaagcagcctc

SEQ ID NO: 180 - DNA, 22 bp, synthetic construct
  source 1..22: mol_type = other DNA
gatgacaatagcaggatcaaac

SEQ ID NO: 181 - DNA, 19 bp, synthetic construct
  source 1..19: mol_type = other DNA
ggttatcctggtaccagca

SEQ ID NO: 182 - DNA, 20 bp, synthetic construct
  source 1..20: mol_type = other DNA
catctactggtaccgacagg

SEQ ID NO: 183 - DNA, 15 bp, synthetic construct
  source 1..15: mol_type = other DNA
ggcgagcaggtggag

SEQ ID NO: 184 - DNA, 21 bp, synthetic construct
  source 1..21: mol_type = other DNA
tctgstctgagatgcaatttt

SEQ ID NO: 185 - DNA, 26 bp, synthetic construct
  source 1..26: mol_type = other DNA
ggctacttcccttggtataagcaaga

SEQ ID NO: 186 - DNA, 20 bp, synthetic construct
  source 1..20: mol_type = other DNA
cagatgcaaggtcaagtgac

SEQ ID NO: 187 - DNA, 18 bp, synthetic construct
  source 1..18: mol_type = other DNA
aaggtccacagctccttc

SEQ ID NO: 188 - DNA, 21 bp, synthetic construct
  source 1..21: mol_type = other DNA
gttctggtatgtgcagtatcc

SEQ ID NO: 189 - DNA, 20 bp, synthetic construct
  source 1..20: mol_type = other DNA
agattccgtgactcaaacag

SEQ ID NO: 190 - DNA, 25 bp, synthetic construct
  source 1..25: mol_type = other DNA
agaaggtrcagcagagcccagaatc

SEQ ID NO: 191 - DNA, 17 bp, synthetic construct
  source 1..17: mol_type = other DNA
gagcrtccasgagggtg

SEQ ID NO: 192 - DNA, 18 bp, synthetic construct
  source 1..18: mol_type = other DNA
ccagtggttcaaggagtg

SEQ ID NO: 193 - DNA, 19 bp, synthetic construct
  source 1..19: mol_type = other DNA
agagaaggtcgagcaacac

SEQ ID NO: 194 - DNA, 18 bp, synthetic construct
  source 1..18: mol_type = other DNA
aagacccaagtggagcag

SEQ ID NO: 195 - DNA, 17 bp, synthetic construct
  source 1..17: mol_type = other DNA
tgacccagacagaaggc

SEQ ID NO: 196 - DNA, 16 bp, synthetic construct
  source 1..16: mol_type = other DNA
tccttggttctgcagg

SEQ ID NO: 197 - DNA, 18 bp, synthetic construct
  source 1..18: mol_type = other DNA
gcagcaggtgagacaaag

SEQ ID NO: 198 - DNA, 22 bp, synthetic construct
  source 1..22: mol_type = other DNA
cascttyttagtggagagatgg

SEQ ID NO: 199 - DNA, 20 bp, synthetic construct
  source 1..20: mol_type = other DNA
gtacaagcaaacagcaagtg

SEQ ID NO: 200 - DNA, 16 bp, synthetic construct
  source 1..16: mol_type = other DNA
cagtccgtggaccagc

SEQ ID NO: 201 - DNA, 16 bp, synthetic construct
  source 1..16: mol_type = other DNA
aacggctggagcagag

SEQ ID NO: 202 - DNA, 21 bp, synthetic construct
  source 1..21: mol_type = other DNA
gcaagttaaacaaagctctcc

SEQ ID NO: 203 - DNA, 18 bp, synthetic construct
  source 1..18: mol_type = other DNA
gtgcacttgccttgtagc

SEQ ID NO: 204-229 - skipped (26 entries)

SEQ ID NO: 230 - DNA, 20 bp, synthetic construct
  source 1..20: mol_type = other DNA
cttttctttccaatacaccc

SEQ ID NO: 231 - DNA, 18 bp, synthetic construct
  source 1..18: mol_type = other DNA
acccaaatgttgcatcag

SEQ ID NO: 232 - DNA, 16 bp, synthetic construct
  source 1..16: mol_type = other DNA
tctgtgcaggtggcag

SEQ ID NO: 233 - DNA, 21 bp, synthetic construct
  source 1..21: mol_type = other DNA
tgtatatttggaaccagttgc

SEQ ID NO: 234 - DNA, 16 bp, synthetic construct
  source 1..16: mol_type = other DNA
catcacgctgacccag

SEQ ID NO: 235 - DNA, 22 bp, synthetic construct
  source 1..22: mol_type = other DNA
cascttyttagtggagagatgg

SEQ ID NO: 236 - DNA, 16 bp, synthetic construct
  source 1..16: mol_type = other DNA
tccttggttctgcagg

SEQ ID NO: 237 - DNA, 18 bp, synthetic construct
  source 1..18: mol_type = other DNA
gcagcaggtgagacaaag

SEQ ID NO: 238 - DNA, 20 bp, synthetic construct
  source 1..20: mol_type = other DNA
cagatgcaaggtcaagtgac

SEQ ID NO: 239 - DNA, 18 bp, synthetic construct
  source 1..18: mol_type = other DNA
aaggtccacagctccttc

SEQ ID NO: 240 - DNA, 21 bp, synthetic construct
  source 1..21: mol_type = other DNA
gttctggtatgtgcagtatcc

SEQ ID NO: 241 - DNA, 21 bp, synthetic construct
  source 1..21: mol_type = other DNA
tctgstctgagatgcaatttt

SEQ ID NO: 242 - DNA, 20 bp, synthetic construct
  source 1..20: mol_type = other DNA
gtacaagcaaacagcaagtg

SEQ ID NO: 243 - DNA, 18 bp, synthetic construct
  source 1..18: mol_type = other DNA
gtgcacttgccttgtagc

SEQ ID NO: 244 - DNA, 23 bp, synthetic construct
  source 1..23: mol_type = other DNA
tgaaagaattttgcatatggttc

SEQ ID NO: 245 - DNA, 17 bp, synthetic construct
  source 1..17: mol_type = other DNA
ctccacattcctgagcc

SEQ ID NO: 246 - DNA, 16 bp, synthetic construct
  source 1..16: mol_type = other DNA
actctgagcctgccct

SEQ ID NO: 247 - DNA, 16 bp, synthetic construct
  source 1..16: mol_type = other DNA
gccctcctcacctgag

SEQ ID NO: 248 - DNA, 22 bp, synthetic construct
  source 1..22: mol_type = other DNA
  modified_base 3: mod_base = i
  modified_base 5: mod_base = i
ggntnmaggaacaaaggagaat

SEQ ID NO: 249 - DNA, 23 bp, synthetic construct
  source 1..23: mol_type = other DNA
atycgttcaaatatggaaagaaa

SEQ ID NO: 250 - DNA, 18 bp, synthetic construct
  source 1..18: mol_type = other DNA
ggagaaggtccacagctc

SEQ ID NO: 251 - DNA, 18 bp, synthetic construct
  source 1..18: mol_type = other DNA
caactgccaacaacaagg

SEQ ID NO: 252 - DNA, 19 bp, synthetic construct
  source 1..19: mol_type = other DNA
tccttccacttgcagaaag

SEQ ID NO: 253 - DNA, 15 bp, synthetic construct
  source 1..15: mol_type = other DNA
acggctggccagaag

SEQ ID NO: 254 - DNA, 21 bp, synthetic construct
  source 1..21: mol_type = other DNA
cakgrcytcyytcaactgcac

SEQ ID NO: 255 - DNA, 17 bp, synthetic construct
  source 1..17: mol_type = other DNA
agagccacccttgacac

SEQ ID NO: 256 - DNA, 19 bp, synthetic construct
  source 1..19: mol_type = other DNA
gcttygaggctgagttcag

SEQ ID NO: 257 - DNA, 21 bp, synthetic construct
  source 1..21: mol_type = other DNA
ctacactgagtgttcgagagg

SEQ ID NO: 258 - DNA, 18 bp, synthetic construct
  source 1..18: mol_type = other DNA
aacaggacacaggcaaag

SEQ ID NO: 259 - DNA, 15 bp, synthetic construct
  source 1..15: mol_type = other DNA
ggttccacgccactc

SEQ ID NO: 260 - DNA, 15 bp, synthetic construct
  source 1..15: mol_type = other DNA
tgcaggagggggaga

SEQ ID NO: 261 - DNA, 19 bp, synthetic construct
  source 1..19: mol_type = other DNA
ctctgacagtctgggaagg

SEQ ID NO: 262 - DNA, 24 bp, synthetic construct
  source 1..24: mol_type = other DNA
aytctgtagtcttccagaaatcac

SEQ ID NO: 263 - DNA, 24 bp, synthetic construct
  source 1..24: mol_type = other DNA
attattctctgaactttcagaagc

SEQ ID NO: 264 - DNA, 18 bp, synthetic construct
  source 1..18: mol_type = other DNA
tatgaaggagcctccctg

SEQ ID NO: 265 - DNA, 17 bp, synthetic construct
  source 1..17: mol_type = other DNA
caagatttcaccgcacg

SEQ ID NO: 266 - DNA, 19 bp, synthetic construct
  source 1..19: mol_type = other DNA
gctgactgttcaagaggga

SEQ ID NO: 267 - DNA, 20 bp, synthetic construct
  source 1..20: mol_type = other DNA
aatagtatggctttcctggc

SEQ ID NO: 268 - DNA, 19 bp, synthetic construct
  source 1..19: mol_type = other DNA
gcacattgatttgggagtc

SEQ ID NO: 269 - DNA, 18 bp, synthetic construct
  source 1..18: mol_type = other DNA
gtatccctggatgagctg

SEQ ID NO: 270 - DNA, 18 bp, synthetic construct
  source 1..18: mol_type = other DNA
ggacaatcagactgcctc

SEQ ID NO: 271 - DNA, 18 bp, synthetic construct
  source 1..18: mol_type = other DNA
gatatggggcagatggtg

SEQ ID NO: 272 - DNA, 18 bp, synthetic construct
  source 1..18: mol_type = other DNA
caggtgggaaatgaagtg

SEQ ID NO: 273 - DNA, 19 bp, synthetic construct
  source 1..19: mol_type = other DNA
gccagagctcatgtttctc

SEQ ID NO: 274 - DNA, 18 bp, synthetic construct
  source 1..18: mol_type = other DNA
ccagcagattctcagtcc

SEQ ID NO: 275 - DNA, 19 bp, synthetic construct
  source 1..19: mol_type = other DNA
gtactggtatcggcaggac

SEQ ID NO: 276 - DNA, 18 bp, synthetic construct
  source 1..18: mol_type = other DNA
ggtatcagcagcccagag

SEQ ID NO: 277 - DNA, 18 bp, synthetic construct
  source 1..18: mol_type = other DNA
gtgtgagccagtttcagg

SEQ ID NO: 278 - DNA, 19 bp, synthetic construct
  source 1..19: mol_type = other DNA
gaagcaactctgtggtgtg

SEQ ID NO: 279 - DNA, 18 bp, synthetic construct
  source 1..18: mol_type = other DNA
gaacagggaagctgacac

SEQ ID NO: 280 - DNA, 18 bp, synthetic construct
  source 1..18: mol_type = other DNA
ggtaccgacaggattcag

SEQ ID NO: 281 - DNA, 18 bp, synthetic construct
  source 1..18: mol_type = other DNA
gcttggtatcgtcaatcg

SEQ ID NO: 282 - DNA, 17 bp, synthetic construct
  source 1..17: mol_type = other DNA
gccaggaagcagagatg

SEQ ID NO: 283 - DNA, 19 bp, synthetic construct
  source 1..19: mol_type = other DNA
gcacactgccttttactgg

SEQ ID NO: 284 - DNA, 20 bp, synthetic construct
  source 1..20: mol_type = other DNA
gaggtgtatccctgaaaagg

SEQ ID NO: 285 - DNA, 21 bp, synthetic construct
  source 1..21: mol_type = other DNA
gtactggtatcgacaagaccc

SEQ ID NO: 286 - DNA, 19 bp, synthetic construct
  source 1..19: mol_type = other DNA
ggacatctgtcaaagtggc

SEQ ID NO: 287 - DNA, 19 bp, synthetic construct
  source 1..19: mol_type = other DNA
ctgttggccaggtagagtc

SEQ ID NO: 288 - DNA, 21 bp, synthetic construct
  source 1..21: mol_type = other DNA
gggtagccttttgtttgtttg

SEQ ID NO: 289 - DNA, 19 bp, synthetic construct
  source 1..19: mol_type = other DNA
ctgaattatcggtcaccag

SEQ ID NO: 290 - DNA, 31 bp, synthetic construct
  source 1..31: mol_type = other DNA
gtttagagtttctattatatgtccttgcaac

SEQ ID NO: 291 - DNA, 21 bp, synthetic construct
  source 1..21: mol_type = other DNA
tacccgaagaccaaacaagac

SEQ ID NO: 292 - DNA, 19 bp, synthetic construct
  source 1..19: mol_type = other DNA
agaggaaaggaaatacggc

SEQ ID NO: 293 - DNA, 18 bp, synthetic construct
  source 1..18: mol_type = other DNA
caccaagctagaggggtc

SEQ ID NO: 294 - DNA, 21 bp, synthetic construct
  source 1..21: mol_type = other DNA
tcdggaaagaacttttcaagg

SEQ ID NO: 295 - DNA, 21 bp, synthetic construct
  source 1..21: mol_type = other DNA
gtctctgacaatccaagaagg

SEQ ID NO: 296 - DNA, 18 bp, synthetic construct
  source 1..18: mol_type = other DNA
ccctggactgcacctatg

SEQ ID NO: 297 - DNA, 20 bp, synthetic construct
  source 1..20: mol_type = other DNA
gatcctgcctccttctactg

SEQ ID NO: 298 - DNA, 18 bp, synthetic construct
  source 1..18: mol_type = other DNA
gctccactgaccagacag

SEQ ID NO: 299 - DNA, 24 bp, synthetic construct
  source 1..24: mol_type = other DNA
aytctgtagtcttccagaaatcac

SEQ ID NO: 300 - DNA, 15 bp, synthetic construct
  source 1..15: mol_type = other DNA
tgcaggagggggaga

SEQ ID NO: 301 - DNA, 19 bp, synthetic construct
  source 1..19: mol_type = other DNA
ctctgacagtctgggaagg

SEQ ID NO: 302 - DNA, 18 bp, synthetic construct
  source 1..18: mol_type = other DNA
ggagaaggtccacagctc

SEQ ID NO: 303 - DNA, 18 bp, synthetic construct
  source 1..18: mol_type = other DNA
caactgccaacaacaagg

SEQ ID NO: 304 - DNA, 19 bp, synthetic construct
  source 1..19: mol_type = other DNA
tccttccacttgcagaaag

SEQ ID NO: 305 - DNA, 22 bp, synthetic construct
  source 1..22: mol_type = other DNA
  modified_base 3: mod_base = i
  modified_base 5: mod_base = i
ggntnmaggaacaaaggagaat

SEQ ID NO: 306 - DNA, 24 bp, synthetic construct
  source 1..24: mol_type = other DNA
attattctctgaactttcagaagc

SEQ ID NO: 307 - DNA, 20 bp, synthetic construct
  source 1..20: mol_type = other DNA
aatagtatggctttcctggc

SEQ ID NO: 308 - DNA, 21 bp, synthetic construct
  source 1..21: mol_type = other DNA
gagatgactatagcagggtcg

SEQ ID NO: 309 - DNA, 20 bp, synthetic construct
  source 1..20: mol_type = other DNA
atmaactgcacgtaccagac

SEQ ID NO: 310 - DNA, 21 bp, synthetic construct
  source 1..21: mol_type = other DNA
caccctgacctgcaactacac

SEQ ID NO: 311 - DNA, 18 bp, synthetic construct
  source 1..18: mol_type = other DNA
ctatcaggggccacccag

SEQ ID NO: 312 - DNA, 20 bp, synthetic construct
  source 1..20: mol_type = other DNA
gtgcaactattcctatagtg

SEQ ID NO: 313 - DNA, 19 bp, synthetic construct
  source 1..19: mol_type = other DNA
gtykctgtctctgaaggag

SEQ ID NO: 314 - DNA, 17 bp, synthetic construct
  source 1..17: mol_type = other DNA
ccacccwggttaaaggc

SEQ ID NO: 315 - DNA, 20 bp, synthetic construct
  source 1..20: mol_type = other DNA
ctgctgaggtgcaactactc

SEQ ID NO: 316 - DNA, 17 bp, synthetic construct
  source 1..17: mol_type = other DNA
gtctccagcttctcctg

SEQ ID NO: 317 - DNA, 19 bp, synthetic construct
  source 1..19: mol_type = other DNA
cagtgcctcaractacttc

SEQ ID NO: 318 - DNA, 19 bp, synthetic construct
  source 1..19: mol_type = other DNA
agtggaactgaagtgctac

SEQ ID NO: 319 - DNA, 20 bp, synthetic construct
  source 1..20: mol_type = other DNA
aataccccaaccaaggtctc

SEQ ID NO: 320 - DNA, 19 bp, synthetic construct
  source 1..19: mol_type = other DNA
aggtaactcaagctcagac

SEQ ID NO: 321 - DNA, 20 bp, synthetic construct
  source 1..20: mol_type = other DNA
cacagtcagcggcttaagag

SEQ ID NO: 322 - DNA, 19 bp, synthetic construct
  source 1..19: mol_type = other DNA
gtgcttatgagaacagtgc

SEQ ID NO: 323 - DNA, 19 bp, synthetic construct
  source 1..19: mol_type = other DNA
ccagtcactgcatgttcag

SEQ ID NO: 324 - DNA, 21 bp, synthetic construct
  source 1..21: mol_type = other DNA
cggtttctaagcatccaagag

SEQ ID NO: 325 - DNA, 20 bp, synthetic construct
  source 1..20: mol_type = other DNA
gcgagttaagcaaaatccac

SEQ ID NO: 326 - DNA, 19 bp, synthetic construct
  source 1..19: mol_type = other DNA
gagcagtgaagacaaggtg

SEQ ID NO: 327 - DNA, 19 bp, synthetic construct
  source 1..19: mol_type = other DNA
tgcacatatgacaccagtg

SEQ ID NO: 328 - DNA, 18 bp, synthetic construct
  source 1..18: mol_type = other DNA
ctgttcctgagcacgcag

SEQ ID NO: 329 - DNA, 18 bp, synthetic construct
  source 1..18: mol_type = other DNA
aagtggagcagagtcctc

SEQ ID NO: 330 - DNA, 18 bp, synthetic construct
  source 1..18: mol_type = other DNA
acctcatgtctagcacag

SEQ ID NO: 331 - DNA, 21 bp, synthetic construct
  source 1..21: mol_type = other DNA
gcacccacayttctktcttac

SEQ ID NO: 332 - DNA, 23 bp, synthetic construct
  source 1..23: mol_type = other DNA
attatatcatgtggtaccaacag

SEQ ID NO: 333 - DNA, 19 bp, synthetic construct
  source 1..19: mol_type = other DNA
ggtataagcaagaacctgg

SEQ ID NO: 334 - DNA, 20 bp, synthetic construct
  source 1..20: mol_type = other DNA
gaggctgaatttaagaagag

SEQ ID NO: 335 - DNA, 20 bp, synthetic construct
  source 1..20: mol_type = other DNA
gccaaagccttgagcttctc

SEQ ID NO: 336 - DNA, 21 bp, synthetic construct
  source 1..21: mol_type = other DNA
gytttgaggctgaatttaaga

SEQ ID NO: 337 - DNA, 20 bp, synthetic construct
  source 1..20: mol_type = other DNA
agagtgaaacytccttccac

SEQ ID NO: 338 - DNA, 19 bp, synthetic construct
  source 1..19: mol_type = other DNA
aaccaaggdctccrgcttc

SEQ ID NO: 339 - DNA, 19 bp, synthetic construct
  source 1..19: mol_type = other DNA
atcaawggttttgaggctg

SEQ ID NO: 340 - DNA, 21 bp, synthetic construct
  source 1..21: mol_type = other DNA
aagatggaagrtttacagcac

SEQ ID NO: 341 - DNA, 20 bp, synthetic construct
  source 1..20: mol_type = other DNA
cttattcagacagtgcctca

SEQ ID NO: 342 - DNA, 21 bp, synthetic construct
  source 1..21: mol_type = other DNA
caatgaaacatctctctctgc

SEQ ID NO: 343 - DNA, 20 bp, synthetic construct
  source 1..20: mol_type = other DNA
gtccagtaccccaaacaacg

SEQ ID NO: 344 - DNA, 19 bp, synthetic construct
  source 1..19: mol_type = other DNA
tgaaacctccttccacctg

SEQ ID NO: 345 - DNA, 21 bp, synthetic construct
  source 1..21: mol_type = other DNA
ctgccactgtgaacaatttgc

SEQ ID NO: 346 - DNA, 21 bp, synthetic construct
  source 1..21: mol_type = other DNA
tgcattattgatagccataag

SEQ ID NO: 347 - DNA, 21 bp, synthetic construct
  source 1..21: mol_type = other DNA
tgccttgcactggtacagatg

SEQ ID NO: 348 - DNA, 18 bp, synthetic construct
  source 1..18: mol_type = other DNA
cgacagattcactccgag

SEQ ID NO: 349 - DNA, 20 bp, synthetic construct
  source 1..20: mol_type = other DNA
tacacttgccttgtaaccac

SEQ ID NO: 350 - DNA, 20 bp, synthetic construct
  source 1..20: mol_type = other DNA
tcactgcgtactgcaactcc

SEQ ID NO: 351 - DNA, 17 bp, synthetic construct
  source 1..17: mol_type = other DNA
agaagcatggtgaagcg

SEQ ID NO: 352 - DNA, 19 bp, synthetic construct
  source 1..19: mol_type = other DNA
atctcaccataaactgcac

SEQ ID NO: 353 - DNA, 21 bp, synthetic construct
  source 1..21: mol_type = other DNA
tcaaccgcttcagacagactg

SEQ ID NO: 354 - DNA, 20 bp, synthetic construct
  source 1..20: mol_type = other DNA
gtgcaattattctgccactg

SEQ ID NO: 355 - DNA, 18 bp, synthetic construct
  source 1..18: mol_type = other DNA
atccttgctttgtgacac

SEQ ID NO: 356 - DNA, 41 bp, synthetic construct
  source 1..41: mol_type = other DNA
acaccttgttcaggtcctccggagatgtgtataagagacag

SEQ ID NO: 357 - DNA, 32 bp, synthetic construct
  source 1..32: mol_type = other DNA
acacgttgttcaggtcctctttcttatatggg

SEQ ID NO: 358 - DNA, 42 bp, synthetic construct
  source 1..42: mol_type = other DNA
agtcacatttctcagatcctcggagatgtgtataagagacag

SEQ ID NO: 359 - DNA, 33 bp, synthetic construct
  source 1..33: mol_type = other DNA
agtcacatttctcagatccttttcttatatggg

SEQ ID NO: 360 - DNA, 41 bp, synthetic construct
  source 1..41: mol_type = other DNA
cgactcaagtgtgtggaacagtgggtcagggttctggatat

SEQ ID NO: 361 - DNA, 41 bp, synthetic construct
  source 1..41: mol_type = other DNA
cgactcaagtgtgtggaacatggggtcagggttctggatat

SEQ ID NO: 362 - DNA, 41 bp, synthetic construct
  source 1..41: mol_type = other DNA
cgactcaagtgtgtggaacgatgggtcagggttctggatat

SEQ ID NO: 363 - DNA, 41 bp, synthetic construct
  source 1..41: mol_type = other DNA
cgactcaagtgtgtggaacgtagggtcagggttctggatat

SEQ ID NO: 364 - DNA, 41 bp, synthetic construct
  source 1..41: mol_type = other DNA
cgactcaagtgtgtggaactaggggtcagggttctggatat

SEQ ID NO: 365 - DNA, 41 bp, synthetic construct
  source 1..41: mol_type = other DNA
cgactcaagtgtgtggaactgagggtcagggttctggatat

SEQ ID NO: 366 - DNA, 41 bp, synthetic construct
  source 1..41: mol_type = other DNA
cgactcaagtgtgtggaagactgggtcagggttctggatat

SEQ ID NO: 367 - DNA, 41 bp, synthetic construct
  source 1..41: mol_type = other DNA
cgactcaagtgtgtggaagatcgggtcagggttctggatat

SEQ ID NO: 368 - DNA, 41 bp, synthetic construct
  source 1..41: mol_type = other DNA
cgactcaagtgtgtggaagcatgggtcagggttctggatat

SEQ ID NO: 369 - DNA, 41 bp, synthetic construct
  source 1..41: mol_type = other DNA
cgactcaagtgtgtggaagctagggtcagggttctggatat

SEQ ID NO: 370 - DNA, 41 bp, synthetic construct
  source 1..41: mol_type = other DNA
cgactcaagtgtgtggaagtacgggtcagggttctggatat

SEQ ID NO: 371 - DNA, 41 bp, synthetic construct
  source 1..41: mol_type = other DNA
cgactcaagtgtgtggaagtcagggtcagggttctggatat

SEQ ID NO: 372 - DNA, 41 bp, synthetic construct
  source 1..41: mol_type = other DNA
cgactcaagtgtgtggaatacggggtcagggttctggatat

SEQ ID NO: 373 - DNA, 41 bp, synthetic construct
  source 1..41: mol_type = other DNA
cgactcaagtgtgtggaatagcgggtcagggttctggatat

SEQ ID NO: 374 - DNA, 41 bp, synthetic construct
  source 1..41: mol_type = other DNA
cgactcaagtgtgtggaatcaggggtcagggttctggatat

SEQ ID NO: 375 - DNA, 41 bp, synthetic construct
  source 1..41: mol_type = other DNA
cgactcaagtgtgtggaatcgagggtcagggttctggatat

SEQ ID NO: 376 - DNA, 41 bp, synthetic construct
  source 1..41: mol_type = other DNA
cgactcaagtgtgtggaatgacgggtcagggttctggatat

SEQ ID NO: 377 - DNA, 41 bp, synthetic construct
  source 1..41: mol_type = other DNA
cgactcaagtgtgtggaatgcagggtcagggttctggatat

SEQ ID NO: 378 - DNA, 41 bp, synthetic construct
  source 1..41: mol_type = other DNA
cgactcaagtgtgtggacaagtgggtcagggttctggatat

SEQ ID NO: 379 - DNA, 41 bp, synthetic construct
  source 1..41: mol_type = other DNA
cgactcaagtgtgtggacaatggggtcagggttctggatat

SEQ ID NO: 380 - DNA, 41 bp, synthetic construct
  source 1..41: mol_type = other DNA
cgactcaagtgtgtggacagatgggtcagggttctggatat

SEQ ID NO: 381 - DNA, 41 bp, synthetic construct
  source 1..41: mol_type = other DNA
cgactcaagtgtgtggacagtagggtcagggttctggatat

SEQ ID NO: 382 - DNA, 41 bp, synthetic construct
  source 1..41: mol_type = other DNA
cgactcaagtgtgtggacataggggtcagggttctggatat

SEQ ID NO: 383 - DNA, 41 bp, synthetic construct
  source 1..41: mol_type = other DNA
cgactcaagtgtgtggacatgagggtcagggttctggatat

SEQ ID NO: 384 - DNA, 41 bp, synthetic construct
  source 1..41: mol_type = other DNA
cgactcaagtgtgtggaccgctgggtcagggttctggatat

SEQ ID NO: 385 - DNA, 41 bp, synthetic construct
  source 1..41: mol_type = other DNA
cgactcaagtgtgtggaccgtcgggtcagggttctggatat

SEQ ID NO: 386 - DNA, 41 bp, synthetic construct
  source 1..41: mol_type = other DNA
cgactcaagtgtgtggacctcggggtcagggttctggatat

SEQ ID NO: 387 - DNA, 41 bp, synthetic construct
  source 1..41: mol_type = other DNA
cgactcaagtgtgtggacctgcgggtcagggttctggatat

SEQ ID NO: 388 - DNA, 41 bp, synthetic construct
  source 1..41: mol_type = other DNA
cgactcaagtgtgtggacgaatgggtcagggttctggatat

SEQ ID NO: 389 - DNA, 41 bp, synthetic construct
  source 1..41: mol_type = other DNA
cgactcaagtgtgtggacgatagggtcagggttctggatat

SEQ ID NO: 390 - DNA, 41 bp, synthetic construct
  source 1..41: mol_type = other DNA
cgactcaagtgtgtggacgcctgggtcagggttctggatat

SEQ ID NO: 391 - DNA, 41 bp, synthetic construct
  source 1..41: mol_type = other DNA
cgactcaagtgtgtggacgctcgggtcagggttctggatat

SEQ ID NO: 392 - DNA, 41 bp, synthetic construct
  source 1..41: mol_type = other DNA
cgactcaagtgtgtggacggtggggtcagggttctggatat

SEQ ID NO: 393 - DNA, 41 bp, synthetic construct
  source 1..41: mol_type = other DNA
cgactcaagtgtgtggacgtaagggtcagggttctggatat

SEQ ID NO: 394 - DNA, 41 bp, synthetic construct
  source 1..41: mol_type = other DNA
cgactcaagtgtgtggacgtccgggtcagggttctggatat

SEQ ID NO: 395 - DNA, 41 bp, synthetic construct
  source 1..41: mol_type = other DNA
cgactcaagtgtgtggacgtgggggtcagggttctggatat

SEQ ID NO: 396 - DNA, 41 bp, synthetic construct
  source 1..41: mol_type = other DNA
cgactcaagtgtgtggactaaggggtcagggttctggatat

SEQ ID NO: 397 - DNA, 41 bp, synthetic construct
  source 1..41: mol_type = other DNA
cgactcaagtgtgtggactagagggtcagggttctggatat

SEQ ID NO: 398 - DNA, 41 bp, synthetic construct
  source 1..41: mol_type = other DNA
cgactcaagtgtgtggactccggggtcagggttctggatat

SEQ ID NO: 399 - DNA, 41 bp, synthetic construct
  source 1..41: mol_type = other DNA
cgactcaagtgtgtggactcgcgggtcagggttctggatat

SEQ ID NO: 400 - DNA, 41 bp, synthetic construct
  source 1..41: mol_type = other DNA
cgactcaagtgtgtggactgaagggtcagggttctggatat

SEQ ID NO: 401 - DNA, 41 bp, synthetic construct
  source 1..41: mol_type = other DNA
cgactcaagtgtgtggactgccgggtcagggttctggatat

SEQ ID NO: 402 - DNA, 41 bp, synthetic construct
  source 1..41: mol_type = other DNA
cgactcaagtgtgtggactgttgggtcagggttctggatat

SEQ ID NO: 403 - DNA, 41 bp, synthetic construct
  source 1..41: mol_type = other DNA
cgactcaagtgtgtggacttgtgggtcagggttctggatat

SEQ ID NO: 404 - DNA, 41 bp, synthetic construct
  source 1..41: mol_type = other DNA
cgactcaagtgtgtggagaactgggtcagggttctggatat

SEQ ID NO: 405 - DNA, 41 bp, synthetic construct
  source 1..41: mol_type = other DNA
cgactcaagtgtgtggagaatcgggtcagggttctggatat

SEQ ID NO: 406 - DNA, 41 bp, synthetic construct
  source 1..41: mol_type = other DNA
cgactcaagtgtgtggagacatgggtcagggttctggatat

SEQ ID NO: 407 - DNA, 41 bp, synthetic construct
  source 1..41: mol_type = other DNA
cgactcaagtgtgtggagactagggtcagggttctggatat

SEQ ID NO: 408 - DNA, 41 bp, synthetic construct
  source 1..41: mol_type = other DNA
cgactcaagtgtgtggagatacgggtcagggttctggatat

SEQ ID NO: 409 - DNA, 41 bp, synthetic construct
  source 1..41: mol_type = other DNA
cgactcaagtgtgtggagatcagggtcagggttctggatat

SEQ ID NO: 410 - DNA, 41 bp, synthetic construct
  source 1..41: mol_type = other DNA
cgactcaagtgtgtggagcaatgggtcagggttctggatat

SEQ ID NO: 411 - DNA, 41 bp, synthetic construct
  source 1..41: mol_type = other DNA
cgactcaagtgtgtggagcatagggtcagggttctggatat

SEQ ID NO: 412 - DNA, 41 bp, synthetic construct
  source 1..41: mol_type = other DNA
cgactcaagtgtgtggagcctcgggtcagggttctggatat

SEQ ID NO: 413 - DNA, 41 bp, synthetic construct
  source 1..41: mol_type = other DNA
cgactcaagtgtgtggagcggtgggtcagggttctggatat

SEQ ID NO: 414 - DNA, 41 bp, synthetic construct
  source 1..41: mol_type = other DNA
cgactcaagtgtgtggagcgtggggtcagggttctggatat

SEQ ID NO: 415 - DNA, 41 bp, synthetic construct
  source 1..41: mol_type = other DNA
cgactcaagtgtgtggagctaagggtcagggttctggatat

SEQ ID NO: 416 - DNA, 41 bp, synthetic construct
  source 1..41: mol_type = other DNA
cgactcaagtgtgtggagctccgggtcagggttctggatat

SEQ ID NO: 417 - DNA, 41 bp, synthetic construct
  source 1..41: mol_type = other DNA
cgactcaagtgtgtggagctgggggtcagggttctggatat

SEQ ID NO: 418 - DNA, 41 bp, synthetic construct
  source 1..41: mol_type = other DNA
cgactcaagtgtgtggaggcgtgggtcagggttctggatat

SEQ ID NO: 419 - DNA, 41 bp, synthetic construct
  source 1..41: mol_type = other DNA
cgactcaagtgtgtggaggctggggtcagggttctggatat

SEQ ID NO: 420 - DNA, 41 bp, synthetic construct
  source 1..41: mol_type = other DNA
cgactcaagtgtgtggaggtcggggtcagggttctggatat

SEQ ID NO: 421 - DNA, 41 bp, synthetic construct
  source 1..41: mol_type = other DNA
cgactcaagtgtgtggaggtgcgggtcagggttctggatat

SEQ ID NO: 422 - DNA, 41 bp, synthetic construct
  source 1..41: mol_type = other DNA
cgactcaagtgtgtggagtaacgggtcagggttctggatat

SEQ ID NO: 423 - DNA, 41 bp, synthetic construct
  source 1..41: mol_type = other DNA
cgactcaagtgtgtggagtacagggtcagggttctggatat

SEQ ID NO: 424 - DNA, 41 bp, synthetic construct
  source 1..41: mol_type = other DNA
cgactcaagtgtgtggagtcaagggtcagggttctggatat

SEQ ID NO: 425 - DNA, 41 bp, synthetic construct
  source 1..41: mol_type = other DNA
cgactcaagtgtgtggagtcgggggtcagggttctggatat

SEQ ID NO: 426 - DNA, 41 bp, synthetic construct
  source 1..41: mol_type = other DNA
cgactcaagtgtgtggagtcttgggtcagggttctggatat

SEQ ID NO: 427 - DNA, 41 bp, synthetic construct
  source 1..41: mol_type = other DNA
cgactcaagtgtgtggagtgcggggtcagggttctggatat

SEQ ID NO: 428 - DNA, 41 bp, synthetic construct
  source 1..41: mol_type = other DNA
cgactcaagtgtgtggagtggcgggtcagggttctggatat

SEQ ID NO: 429 - DNA, 41 bp, synthetic construct
  source 1..41: mol_type = other DNA
cgactcaagtgtgtggagttctgggtcagggttctggatat

SEQ ID NO: 430 - DNA, 41 bp, synthetic construct
  source 1..41: mol_type = other DNA
cgactcaagtgtgtggataacggggtcagggttctggatat

SEQ ID NO: 431 - DNA, 41 bp, synthetic construct
  source 1..41: mol_type = other DNA
cgactcaagtgtgtggataagcgggtcagggttctggatat

SEQ ID NO: 432 - DNA, 41 bp, synthetic construct
  source 1..41: mol_type = other DNA
cgactcaagtgtgtggatacaggggtcagggttctggatat

SEQ ID NO: 433 - DNA, 41 bp, synthetic construct
  source 1..41: mol_type = other DNA
cgactcaagtgtgtggatacgagggtcagggttctggatat

SEQ ID NO: 434 - DNA, 41 bp, synthetic construct
  source 1..41: mol_type = other DNA
cgactcaagtgtgtggatagacgggtcagggttctggatat

SEQ ID NO: 435 - DNA, 41 bp, synthetic construct
  source 1..41: mol_type = other DNA
cgactcaagtgtgtggatagcagggtcagggttctggatat

SEQ ID NO: 436 - DNA, 41 bp, synthetic construct
  source 1..41: mol_type = other DNA
cgactcaagtgtgtggatcaaggggtcagggttctggatat

SEQ ID NO: 437 - DNA, 41 bp, synthetic construct
  source 1..41: mol_type = other DNA
cgactcaagtgtgtggatcagagggtcagggttctggatat

SEQ ID NO: 438 - DNA, 41 bp, synthetic construct
  source 1..41: mol_type = other DNA
cgactcaagtgtgtggatccgcgggtcagggttctggatat

SEQ ID NO: 439 - DNA, 41 bp, synthetic construct
  source 1..41: mol_type = other DNA
cgactcaagtgtgtggatcgaagggtcagggttctggatat

SEQ ID NO: 440 - DNA, 41 bp, synthetic construct
  source 1..41: mol_type = other DNA
cgactcaagtgtgtggatcgccgggtcagggttctggatat

SEQ ID NO: 441 - DNA, 41 bp, synthetic construct
  source 1..41: mol_type = other DNA
cgactcaagtgtgtggatcgttgggtcagggttctggatat

SEQ ID NO: 442 - DNA, 41 bp, synthetic construct
  source 1..41: mol_type = other DNA
cgactcaagtgtgtggatctgtgggtcagggttctggatat

SEQ ID NO: 443 - DNA, 41 bp, synthetic construct
  source 1..41: mol_type = other DNA
cgactcaagtgtgtggatcttggggtcagggttctggatat

SEQ ID NO: 444 - DNA, 41 bp, synthetic construct
  source 1..41: mol_type = other DNA
cgactcaagtgtgtggatgaacgggtcagggttctggatat

SEQ ID NO: 445 - DNA, 41 bp, synthetic construct
  source 1..41: mol_type = other DNA
cgactcaagtgtgtggatgacagggtcagggttctggatat

SEQ ID NO: 446 - DNA, 41 bp, synthetic construct
  source 1..41: mol_type = other DNA
cgactcaagtgtgtggatgcaagggtcagggttctggatat

SEQ ID NO: 447 - DNA, 41 bp, synthetic construct
  source 1..41: mol_type = other DNA
cgactcaagtgtgtggatgcgggggtcagggttctggatat

SEQ ID NO: 448 - DNA, 41 bp, synthetic construct
  source 1..41: mol_type = other DNA
cgactcaagtgtgtggatgcttgggtcagggttctggatat

SEQ ID NO: 449 - DNA, 41 bp, synthetic construct
  source 1..41: mol_type = other DNA
cgactcaagtgtgtggatggcggggtcagggttctggatat

SEQ ID NO: 450 - DNA, 41 bp, synthetic construct
  source 1..41: mol_type = other DNA
cgactcaagtgtgtggatgtctgggtcagggttctggatat

SEQ ID NO: 451 - DNA, 41 bp, synthetic construct
  source 1..41: mol_type = other DNA
cgactcaagtgtgtggatgttcgggtcagggttctggatat

SEQ ID NO: 452 - DNA, 41 bp, synthetic construct
  source 1..41: mol_type = other DNA
cgactcaagtgtgtggattcgtgggtcagggttctggatat

SEQ ID NO: 453 - DNA, 41 bp, synthetic construct
  source 1..41: mol_type = other DNA
cgactcaagtgtgtggattctggggtcagggttctggatat

SEQ ID NO: 454 - DNA, 41 bp, synthetic construct
  source 1..41: mol_type = other DNA
cgactcaagtgtgtggattgctgggtcagggttctggatat

SEQ ID NO: 455 - DNA, 41 bp, synthetic construct
  source 1..41: mol_type = other DNA
cgactcaagtgtgtggattgtcgggtcagggttctggatat

SEQ ID NO: 456 - DNA, 41 bp, synthetic construct
  source 1..41: mol_type = other DNA
cgactcagattggtacaacagtacacsttkttcaggtcctc

SEQ ID NO: 457 - DNA, 41 bp, synthetic construct
  source 1..41: mol_type = other DNA
cgactcagattggtacaacatgacacsttkttcaggtcctc

SEQ ID NO: 458 - DNA, 41 bp, synthetic construct
  source 1..41: mol_type = other DNA
cgactcagattggtacaacgatacacsttkttcaggtcctc

SEQ ID NO: 459 - DNA, 41 bp, synthetic construct
  source 1..41: mol_type = other DNA
cgactcagattggtacaacgtaacacsttkttcaggtcctc

SEQ ID NO: 460 - DNA, 41 bp, synthetic construct
  source 1..41: mol_type = other DNA
cgactcagattggtacaactagacacsttkttcaggtcctc

SEQ ID NO: 461 - DNA, 41 bp, synthetic construct
  source 1..41: mol_type = other DNA
cgactcagattggtacaactgaacacsttkttcaggtcctc

SEQ ID NO: 462 - DNA, 41 bp, synthetic construct
  source 1..41: mol_type = other DNA
cgactcagattggtacaagactacacsttkttcaggtcctc

SEQ ID NO: 463 - DNA, 41 bp, synthetic construct
  source 1..41: mol_type = other DNA
cgactcagattggtacaagatcacacsttkttcaggtcctc

SEQ ID NO: 464 - DNA, 41 bp, synthetic construct
  source 1..41: mol_type = other DNA
cgactcagattggtacaagcatacacsttkttcaggtcctc

SEQ ID NO: 465 - DNA, 41 bp, synthetic construct
  source 1..41: mol_type = other DNA
cgactcagattggtacaagctaacacsttkttcaggtcctc

SEQ ID NO: 466 - DNA, 41 bp, synthetic construct
  source 1..41: mol_type = other DNA
cgactcagattggtacaagtacacacsttkttcaggtcctc

SEQ ID NO: 467 - DNA, 41 bp, synthetic construct
  source 1..41: mol_type = other DNA
cgactcagattggtacaagtcaacacsttkttcaggtcctc

SEQ ID NO: 468 - DNA, 41 bp, synthetic construct
  source 1..41: mol_type = other DNA
cgactcagattggtacaatacgacacsttkttcaggtcctc

SEQ ID NO: 469 - DNA, 41 bp, synthetic construct
  source 1..41: mol_type = other DNA
cgactcagattggtacaatagcacacsttkttcaggtcctc

SEQ ID NO: 470 - DNA, 41 bp, synthetic construct
  source 1..41: mol_type = other DNA
cgactcagattggtacaatcagacacsttkttcaggtcctc

SEQ ID NO: 471 - DNA, 41 bp, synthetic construct
  source 1..41: mol_type = other DNA
cgactcagattggtacaatcgaacacsttkttcaggtcctc

SEQ ID NO: 472 - DNA, 41 bp, synthetic construct
  source 1..41: mol_type = other DNA
cgactcagattggtacaatgacacacsttkttcaggtcctc

SEQ ID NO: 473 - DNA, 41 bp, synthetic construct
  source 1..41: mol_type = other DNA
cgactcagattggtacaatgcaacacsttkttcaggtcctc

SEQ ID NO: 474 - DNA, 41 bp, synthetic construct
  source 1..41: mol_type = other DNA
cgactcagattggtacacaagtacacsttkttcaggtcctc

SEQ ID NO: 475 - DNA, 41 bp, synthetic construct
  source 1..41: mol_type = other DNA
cgactcagattggtacacaatgacacsttkttcaggtcctc

SEQ ID NO: 476 - DNA, 41 bp, synthetic construct
  source 1..41: mol_type = other DNA
cgactcagattggtacacagatacacsttkttcaggtcctc

SEQ ID NO: 477 - DNA, 41 bp, synthetic construct
  source 1..41: mol_type = other DNA
cgactcagattggtacacagtaacacsttkttcaggtcctc

SEQ ID NO: 478 - DNA, 41 bp, synthetic construct
  source 1..41: mol_type = other DNA
cgactcagattggtacacatagacacsttkttcaggtcctc

SEQ ID NO: 479 - DNA, 41 bp, synthetic construct
  source 1..41: mol_type = other DNA
cgactcagattggtacacatgaacacsttkttcaggtcctc

SEQ ID NO: 480 - DNA, 41 bp, synthetic construct
  source 1..41: mol_type = other DNA
cgactcagattggtacaccgctacacsttkttcaggtcctc

SEQ ID NO: 481 - DNA, 41 bp, synthetic construct
  source 1..41: mol_type = other DNA
cgactcagattggtacaccgtcacacsttkttcaggtcctc

SEQ ID NO: 482 - DNA, 41 bp, synthetic construct
  source 1..41: mol_type = other DNA
cgactcagattggtacacctcgacacsttkttcaggtcctc

SEQ ID NO: 483 - DNA, 41 bp, synthetic construct
  source 1..41: mol_type = other DNA
cgactcagattggtacacctgcacacsttkttcaggtcctc

SEQ ID NO: 484 - DNA, 41 bp, synthetic construct
  source 1..41: mol_type = other DNA
cgactcagattggtacacgaatacacsttkttcaggtcctc

SEQ ID NO: 485 - DNA, 41 bp, synthetic construct
  source 1..41: mol_type = other DNA
cgactcagattggtacacgataacacsttkttcaggtcctc

SEQ ID NO: 486 - DNA, 41 bp, synthetic construct
  source 1..41: mol_type = other DNA
cgactcagattggtacacgcctacacsttkttcaggtcctc

SEQ ID NO: 487 - DNA, 41 bp, synthetic construct
  source 1..41: mol_type = other DNA
cgactcagattggtacacgctcacacsttkttcaggtcctc

SEQ ID NO: 488 - DNA, 41 bp, synthetic construct
  source 1..41: mol_type = other DNA
cgactcagattggtacacggtgacacsttkttcaggtcctc

SEQ ID NO: 489 - DNA, 41 bp, synthetic construct
  source 1..41: mol_type = other DNA
cgactcagattggtacacgtaaacacsttkttcaggtcctc

SEQ ID NO: 490 - DNA, 41 bp, synthetic construct
  source 1..41: mol_type = other DNA
cgactcagattggtacacgtccacacsttkttcaggtcctc

SEQ ID NO: 491 - DNA, 41 bp, synthetic construct
  source 1..41: mol_type = other DNA
cgactcagattggtacacgtggacacsttkttcaggtcctc

SEQ ID NO: 492 - DNA, 41 bp, synthetic construct
  source 1..41: mol_type = other DNA
cgactcagattggtacactaagacacsttkttcaggtcctc

SEQ ID NO: 493 - DNA, 41 bp, synthetic construct
  source 1..41: mol_type = other DNA
cgactcagattggtacactagaacacsttkttcaggtcctc

SEQ ID NO: 494 - DNA, 41 bp, synthetic construct
  source 1..41: mol_type = other DNA
cgactcagattggtacactccgacacsttkttcaggtcctc

SEQ ID NO: 495 - DNA, 41 bp, synthetic construct
  source 1..41: mol_type = other DNA
cgactcagattggtacactcgcacacsttkttcaggtcctc

SEQ ID NO: 496 - DNA, 41 bp, synthetic construct
  source 1..41: mol_type = other DNA
cgactcagattggtacactgaaacacsttkttcaggtcctc

SEQ ID NO: 497 - DNA, 41 bp, synthetic construct
  source 1..41: mol_type = other DNA
cgactcagattggtacactgccacacsttkttcaggtcctc

SEQ ID NO: 498 - DNA, 41 bp, synthetic construct
  source 1..41: mol_type = other DNA
cgactcagattggtacactgttacacsttkttcaggtcctc

SEQ ID NO: 499 - DNA, 41 bp, synthetic construct
  source 1..41: mol_type = other DNA
cgactcagattggtacacttgtacacsttkttcaggtcctc

SEQ ID NO: 500 - DNA, 41 bp, synthetic construct
  source 1..41: mol_type = other DNA
cgactcagattggtacagaactacacsttkttcaggtcctc

SEQ ID NO: 501 - DNA, 41 bp, synthetic construct
  source 1..41: mol_type = other DNA
cgactcagattggtacagaatcacacsttkttcaggtcctc

SEQ ID NO: 502 - DNA, 41 bp, synthetic construct
  source 1..41: mol_type = other DNA
cgactcagattggtacagacatacacsttkttcaggtcctc

SEQ ID NO: 503 - DNA, 41 bp, synthetic construct
  source 1..41: mol_type = other DNA
cgactcagattggtacagactaacacsttkttcaggtcctc

SEQ ID NO: 504 - DNA, 41 bp, synthetic construct
  source 1..41: mol_type = other DNA
cgactcagattggtacagatacacacsttkttcaggtcctc

SEQ ID NO: 505 - DNA, 41 bp, synthetic construct
  source 1..41: mol_type = other DNA
cgactcagattggtacagatcaacacsttkttcaggtcctc

SEQ ID NO: 506 - DNA, 41 bp, synthetic construct
  source 1..41: mol_type = other DNA
cgactcagattggtacagcaatacacsttkttcaggtcctc

SEQ ID NO: 507 - DNA, 41 bp, synthetic construct
  source 1..41: mol_type = other DNA
cgactcagattggtacagcataacacsttkttcaggtcctc

SEQ ID NO: 508 - DNA, 41 bp, synthetic construct
  source 1..41: mol_type = other DNA
cgactcagattggtacagcctcacacsttkttcaggtcctc

SEQ ID NO: 509 - DNA, 41 bp, synthetic construct
  source 1..41: mol_type = other DNA
cgactcagattggtacagcggtacacsttkttcaggtcctc

SEQ ID NO: 510 - DNA, 41 bp, synthetic construct
  source 1..41: mol_type = other DNA
cgactcagattggtacagcgtgacacsttkttcaggtcctc

SEQ ID NO: 511 - DNA, 41 bp, synthetic construct
  source 1..41: mol_type = other DNA
cgactcagattggtacagctaaacacsttkttcaggtcctc

SEQ ID NO: 512 - DNA, 41 bp, synthetic construct
  source 1..41: mol_type = other DNA
cgactcagattggtacagctccacacsttkttcaggtcctc

SEQ ID NO: 513 - DNA, 41 bp, synthetic construct
  source 1..41: mol_type = other DNA
cgactcagattggtacagctggacacsttkttcaggtcctc

SEQ ID NO: 514 - DNA, 41 bp, synthetic construct
  source 1..41: mol_type = other DNA
cgactcagattggtacaggcgtacacsttkttcaggtcctc

SEQ ID NO: 515 - DNA, 41 bp, synthetic construct
  source 1..41: mol_type = other DNA
cgactcagattggtacaggctgacacsttkttcaggtcctc

SEQ ID NO: 516 - DNA, 41 bp, synthetic construct
  source 1..41: mol_type = other DNA
cgactcagattggtacaggtcgacacsttkttcaggtcctc

SEQ ID NO: 517 - DNA, 41 bp, synthetic construct
  source 1..41: mol_type = other DNA
cgactcagattggtacaggtgcacacsttkttcaggtcctc

SEQ ID NO: 518 - DNA, 41 bp, synthetic construct
  source 1..41: mol_type = other DNA
cgactcagattggtacagtaacacacsttkttcaggtcctc

SEQ ID NO: 519 - DNA, 41 bp, synthetic construct
  source 1..41: mol_type = other DNA
cgactcagattggtacagtacaacacsttkttcaggtcctc

SEQ ID NO: 520 - DNA, 41 bp, synthetic construct
  source 1..41: mol_type = other DNA
cgactcagattggtacagtcaaacacsttkttcaggtcctc

SEQ ID NO: 521 - DNA, 41 bp, synthetic construct
  source 1..41: mol_type = other DNA
cgactcagattggtacagtcggacacsttkttcaggtcctc

SEQ ID NO: 522 - DNA, 41 bp, synthetic construct
  source 1..41: mol_type = other DNA
cgactcagattggtacagtcttacacsttkttcaggtcctc

SEQ ID NO: 523 - DNA, 41 bp, synthetic construct
  source 1..41: mol_type = other DNA
cgactcagattggtacagtgcgacacsttkttcaggtcctc

SEQ ID NO: 524 - DNA, 41 bp, synthetic construct
  source 1..41: mol_type = other DNA
cgactcagattggtacagtggcacacsttkttcaggtcctc

SEQ ID NO: 525 - DNA, 41 bp, synthetic construct
  source 1..41: mol_type = other DNA
cgactcagattggtacagttctacacsttkttcaggtcctc

SEQ ID NO: 526 - DNA, 41 bp, synthetic construct
  source 1..41: mol_type = other DNA
cgactcagattggtacataacgacacsttkttcaggtcctc

SEQ ID NO: 527 - DNA, 41 bp, synthetic construct
  source 1..41: mol_type = other DNA
cgactcagattggtacataagcacacsttkttcaggtcctc

SEQ ID NO: 528 - DNA, 41 bp, synthetic construct
  source 1..41: mol_type = other DNA
cgactcagattggtacatacagacacsttkttcaggtcctc

SEQ ID NO: 529 - DNA, 41 bp, synthetic construct
  source 1..41: mol_type = other DNA
cgactcagattggtacatacgaacacsttkttcaggtcctc

SEQ ID NO: 530 - DNA, 41 bp, synthetic construct
  source 1..41: mol_type = other DNA
cgactcagattggtacatagacacacsttkttcaggtcctc

SEQ ID NO: 531 - DNA, 41 bp, synthetic construct
  source 1..41: mol_type = other DNA
cgactcagattggtacatagcaacacsttkttcaggtcctc

SEQ ID NO: 532 - DNA, 41 bp, synthetic construct
  source 1..41: mol_type = other DNA
cgactcagattggtacatcaagacacsttkttcaggtcctc

SEQ ID NO: 533 - DNA, 41 bp, synthetic construct
  source 1..41: mol_type = other DNA
cgactcagattggtacatcagaacacsttkttcaggtcctc

SEQ ID NO: 534 - DNA, 41 bp, synthetic construct
  source 1..41: mol_type = other DNA
cgactcagattggtacatccgcacacsttkttcaggtcctc

SEQ ID NO: 535 - DNA, 41 bp, synthetic construct
  source 1..41: mol_type = other DNA
cgactcagattggtacatcgaaacacsttkttcaggtcctc

SEQ ID NO: 536 - DNA, 41 bp, synthetic construct
  source 1..41: mol_type = other DNA
cgactcagattggtacatcgccacacsttkttcaggtcctc

SEQ ID NO: 537 - DNA, 41 bp, synthetic construct
  source 1..41: mol_type = other DNA
cgactcagattggtacatcgttacacsttkttcaggtcctc

SEQ ID NO: 538 - DNA, 41 bp, synthetic construct
  source 1..41: mol_type = other DNA
cgactcagattggtacatctgtacacsttkttcaggtcctc

SEQ ID NO: 539 - DNA, 41 bp, synthetic construct
  source 1..41: mol_type = other DNA
cgactcagattggtacatcttgacacsttkttcaggtcctc

SEQ ID NO: 540 - DNA, 41 bp, synthetic construct
  source 1..41: mol_type = other DNA
cgactcagattggtacatgaacacacsttkttcaggtcctc

SEQ ID NO: 541 - DNA, 41 bp, synthetic construct
  source 1..41: mol_type = other DNA
cgactcagattggtacatgacaacacsttkttcaggtcctc

SEQ ID NO: 542 - DNA, 41 bp, synthetic construct
  source 1..41: mol_type = other DNA
cgactcagattggtacatgcaaacacsttkttcaggtcctc

SEQ ID NO: 543 - DNA, 41 bp, synthetic construct
  source 1..41: mol_type = other DNA
cgactcagattggtacatgcggacacsttkttcaggtcctc

SEQ ID NO: 544 - DNA, 41 bp, synthetic construct
  source 1..41: mol_type = other DNA
cgactcagattggtacatgcttacacsttkttcaggtcctc

SEQ ID NO: 545 - DNA, 41 bp, synthetic construct
  source 1..41: mol_type = other DNA
cgactcagattggtacatggcgacacsttkttcaggtcctc

SEQ ID NO: 546 - DNA, 41 bp, synthetic construct
  source 1..41: mol_type = other DNA
cgactcagattggtacatgtctacacsttkttcaggtcctc

SEQ ID NO: 547 - DNA, 41 bp, synthetic construct
  source 1..41: mol_type = other DNA
cgactcagattggtacatgttcacacsttkttcaggtcctc

SEQ ID NO: 548 - DNA, 41 bp, synthetic construct
  source 1..41: mol_type = other DNA
cgactcagattggtacattcgtacacsttkttcaggtcctc

SEQ ID NO: 549 - DNA, 41 bp, synthetic construct
  source 1..41: mol_type = other DNA
cgactcagattggtacattctgacacsttkttcaggtcctc

SEQ ID NO: 550 - DNA, 41 bp, synthetic construct
  source 1..41: mol_type = other DNA
cgactcagattggtacattgctacacsttkttcaggtcctc

SEQ ID NO: 551 - DNA, 41 bp, synthetic construct
  source 1..41: mol_type = other DNA
cgactcagattggtacattgtcacacsttkttcaggtcctc

SEQ ID NO: 552 - DNA, 39 bp, synthetic construct
  source 1..39: mol_type = other DNA
cgactcaagtgtgtggaacagtttctgggttctggatgt

SEQ ID NO: 553 - DNA, 39 bp, synthetic construct
  source 1..39: mol_type = other DNA
cgactcaagtgtgtggaacatgttctgggttctggatgt

SEQ ID NO: 554 - DNA, 39 bp, synthetic construct
  source 1..39: mol_type = other DNA
cgactcaagtgtgtggaacgatttctgggttctggatgt

SEQ ID NO: 555 - DNA, 39 bp, synthetic construct
  source 1..39: mol_type = other DNA
cgactcaagtgtgtggaacgtattctgggttctggatgt

SEQ ID NO: 556 - DNA, 39 bp, synthetic construct
  source 1..39: mol_type = other DNA
cgactcaagtgtgtggaactagttctgggttctggatgt

SEQ ID NO: 557 - DNA, 39 bp, synthetic construct
  source 1..39: mol_type = other DNA
cgactcaagtgtgtggaactgattctgggttctggatgt

SEQ ID NO: 558 - DNA, 39 bp, synthetic construct
  source 1..39: mol_type = other DNA
cgactcaagtgtgtggaagactttctgggttctggatgt

SEQ ID NO: 559 - DNA, 39 bp, synthetic construct
  source 1..39: mol_type = other DNA
cgactcaagtgtgtggaagatcttctgggttctggatgt

SEQ ID NO: 560 - DNA, 39 bp, synthetic construct
  source 1..39: mol_type = other DNA
cgactcaagtgtgtggaagcatttctgggttctggatgt

SEQ ID NO: 561 - DNA, 39 bp, synthetic construct
  source 1..39: mol_type = other DNA
cgactcaagtgtgtggaagctattctgggttctggatgt

SEQ ID NO: 562 - DNA, 39 bp, synthetic construct
  source 1..39: mol_type = other DNA
cgactcaagtgtgtggaagtacttctgggttctggatgt

SEQ ID NO: 563 - DNA, 39 bp, synthetic construct
  source 1..39: mol_type = other DNA
cgactcaagtgtgtggaagtcattctgggttctggatgt

SEQ ID NO: 564 - DNA, 39 bp, synthetic construct
  source 1..39: mol_type = other DNA
cgactcaagtgtgtggaatacgttctgggttctggatgt

SEQ ID NO: 565 - DNA, 39 bp, synthetic construct
  source 1..39: mol_type = other DNA
cgactcaagtgtgtggaatagcttctgggttctggatgt

SEQ ID NO: 566 - DNA, 39 bp, synthetic construct
  source 1..39: mol_type = other DNA
cgactcaagtgtgtggaatcagttctgggttctggatgt

SEQ ID NO: 567 - DNA, 39 bp, synthetic construct
  source 1..39: mol_type = other DNA
cgactcaagtgtgtggaatcgattctgggttctggatgt

SEQ ID NO: 568 - DNA, 39 bp, synthetic construct
  source 1..39: mol_type = other DNA
cgactcaagtgtgtggaatgacttctgggttctggatgt

SEQ ID NO: 569 - DNA, 39 bp, synthetic construct
  source 1..39: mol_type = other DNA
cgactcaagtgtgtggaatgcattctgggttctggatgt

SEQ ID NO: 570 - DNA, 39 bp, synthetic construct
  source 1..39: mol_type = other DNA
cgactcaagtgtgtggacaagtttctgggttctggatgt

SEQ ID NO: 571 - DNA, 39 bp, synthetic construct
  source 1..39: mol_type = other DNA
cgactcaagtgtgtggacaatgttctgggttctggatgt

SEQ ID NO: 572 - DNA, 39 bp, synthetic construct
  source 1..39: mol_type = other DNA
cgactcaagtgtgtggacagatttctgggttctggatgt

SEQ ID NO: 573 - DNA, 39 bp, synthetic construct
  source 1..39: mol_type = other DNA
cgactcaagtgtgtggacagtattctgggttctggatgt

SEQ ID NO: 574 - DNA, 39 bp, synthetic construct
  source 1..39: mol_type = other DNA
cgactcaagtgtgtggacatagttctgggttctggatgt

SEQ ID NO: 575 - DNA, 39 bp, synthetic construct
  source 1..39: mol_type = other DNA
cgactcaagtgtgtggacatgattctgggttctggatgt

SEQ ID NO: 576 - DNA, 39 bp, synthetic construct
  source 1..39: mol_type = other DNA
cgactcaagtgtgtggaccgctttctgggttctggatgt

SEQ ID NO: 577 - DNA, 39 bp, synthetic construct
  source 1..39: mol_type = other DNA
cgactcaagtgtgtggaccgtcttctgggttctggatgt

SEQ ID NO: 578 - DNA, 39 bp, synthetic construct
  source 1..39: mol_type = other DNA
cgactcaagtgtgtggacctcgttctgggttctggatgt

SEQ ID NO: 579 - DNA, 39 bp, synthetic construct
  source 1..39: mol_type = other DNA
cgactcaagtgtgtggacctgcttctgggttctggatgt

SEQ ID NO: 580 - DNA, 39 bp, synthetic construct
  source 1..39: mol_type = other DNA
cgactcaagtgtgtggacgaatttctgggttctggatgt

SEQ ID NO: 581 - DNA, 39 bp, synthetic construct
  source 1..39: mol_type = other DNA
cgactcaagtgtgtggacgatattctgggttctggatgt

SEQ ID NO: 582 - DNA, 39 bp, synthetic construct
  source 1..39: mol_type = other DNA
cgactcaagtgtgtggacgcctttctgggttctggatgt

SEQ ID NO: 583 - DNA, 39 bp, synthetic construct
  source 1..39: mol_type = other DNA
cgactcaagtgtgtggacgctcttctgggttctggatgt

SEQ ID NO: 584 - DNA, 39 bp, synthetic construct
  source 1..39: mol_type = other DNA
cgactcaagtgtgtggacggtgttctgggttctggatgt

SEQ ID NO: 585 - DNA, 39 bp, synthetic construct
  source 1..39: mol_type = other DNA
cgactcaagtgtgtggacgtaattctgggttctggatgt

SEQ ID NO: 586 - DNA, 39 bp, synthetic construct
  source 1..39: mol_type = other DNA
cgactcaagtgtgtggacgtccttctgggttctggatgt

SEQ ID NO: 587 - DNA, 39 bp, synthetic construct
  source 1..39: mol_type = other DNA
cgactcaagtgtgtggacgtggttctgggttctggatgt

SEQ ID NO: 588 - DNA, 39 bp, synthetic construct
  source 1..39: mol_type = other DNA
cgactcaagtgtgtggactaagttctgggttctggatgt

SEQ ID NO: 589 - DNA, 39 bp, synthetic construct
  source 1..39: mol_type = other DNA
cgactcaagtgtgtggactagattctgggttctggatgt

SEQ ID NO: 590 - DNA, 39 bp, synthetic construct
  source 1..39: mol_type = other DNA
cgactcaagtgtgtggactccgttctgggttctggatgt

SEQ ID NO: 591 - DNA, 39 bp, synthetic construct
  source 1..39: mol_type = other DNA
cgactcaagtgtgtggactcgcttctgggttctggatgt

SEQ ID NO: 592 - DNA, 39 bp, synthetic construct
  source 1..39: mol_type = other DNA
cgactcaagtgtgtggactgaattctgggttctggatgt

SEQ ID NO: 593 - DNA, 39 bp, synthetic construct
  source 1..39: mol_type = other DNA
cgactcaagtgtgtggactgccttctgggttctggatgt

SEQ ID NO: 594 - DNA, 39 bp, synthetic construct
  source 1..39: mol_type = other DNA
cgactcaagtgtgtggactgttttctgggttctggatgt

SEQ ID NO: 595 - DNA, 39 bp, synthetic construct
  source 1..39: mol_type = other DNA
cgactcaagtgtgtggacttgtttctgggttctggatgt

SEQ ID NO: 596 - DNA, 39 bp, synthetic construct
  source 1..39: mol_type = other DNA
cgactcaagtgtgtggagaactttctgggttctggatgt

SEQ ID NO: 597 - DNA, 39 bp, synthetic construct
  source 1..39: mol_type = other DNA
cgactcaagtgtgtggagaatcttctgggttctggatgt

SEQ ID NO: 598 - DNA, 39 bp, synthetic construct
  source 1..39: mol_type = other DNA
cgactcaagtgtgtggagacatttctgggttctggatgt

SEQ ID NO: 599 - DNA, 39 bp, synthetic construct
  source 1..39: mol_type = other DNA
cgactcaagtgtgtggagactattctgggttctggatgt

SEQ ID NO: 600 - DNA, 39 bp, synthetic construct
  source 1..39: mol_type = other DNA
cgactcaagtgtgtggagatacttctgggttctggatgt

SEQ ID NO: 601 - DNA, 39 bp, synthetic construct
  source 1..39: mol_type = other DNA
cgactcaagtgtgtggagatcattctgggttctggatgt

SEQ ID NO: 602 - DNA, 39 bp, synthetic construct
  source 1..39: mol_type = other DNA
cgactcaagtgtgtggagcaatttctgggttctggatgt

SEQ ID NO: 603 - DNA, 39 bp, synthetic construct
  source 1..39: mol_type = other DNA
cgactcaagtgtgtggagcatattctgggttctggatgt

SEQ ID NO: 604 - DNA, 39 bp, synthetic construct
  source 1..39: mol_type = other DNA
cgactcaagtgtgtggagcctcttctgggttctggatgt

SEQ ID NO: 605 - DNA, 39 bp, synthetic construct
  source 1..39: mol_type = other DNA
cgactcaagtgtgtggagcggtttctgggttctggatgt

SEQ ID NO: 606 - DNA, 39 bp, synthetic construct
  source 1..39: mol_type = other DNA
cgactcaagtgtgtggagcgtgttctgggttctggatgt

SEQ ID NO: 607 - DNA, 39 bp, synthetic construct
  source 1..39: mol_type = other DNA
cgactcaagtgtgtggagctaattctgggttctggatgt

SEQ ID NO: 608 - DNA, 39 bp, synthetic construct
  source 1..39: mol_type = other DNA
cgactcaagtgtgtggagctccttctgggttctggatgt

SEQ ID NO: 609 - DNA, 39 bp, synthetic construct
  source 1..39: mol_type = other DNA
cgactcaagtgtgtggagctggttctgggttctggatgt

SEQ ID NO: 610 - DNA, 39 bp, synthetic construct
  source 1..39: mol_type = other DNA
cgactcaagtgtgtggaggcgtttctgggttctggatgt

SEQ ID NO: 611 - DNA, 39 bp, synthetic construct
  source 1..39: mol_type = other DNA
cgactcaagtgtgtggaggctgttctgggttctggatgt

SEQ ID NO: 612 - DNA, 39 bp, synthetic construct
  source 1..39: mol_type = other DNA
cgactcaagtgtgtggaggtcgttctgggttctggatgt

SEQ ID NO: 613 - DNA, 39 bp, synthetic construct
  source 1..39: mol_type = other DNA
cgactcaagtgtgtggaggtgcttctgggttctggatgt

SEQ ID NO: 614 - DNA, 39 bp, synthetic construct
  source 1..39: mol_type = other DNA
cgactcaagtgtgtggagtaacttctgggttctggatgt

SEQ ID NO: 615 - DNA, 39 bp, synthetic construct
  source 1..39: mol_type = other DNA
cgactcaagtgtgtggagtacattctgggttctggatgt

SEQ ID NO: 616 - DNA, 39 bp, synthetic construct
  source 1..39: mol_type = other DNA
cgactcaagtgtgtggagtcaattctgggttctggatgt

SEQ ID NO: 617 - DNA, 39 bp, synthetic construct
  source 1..39: mol_type = other DNA
cgactcaagtgtgtggagtcggttctgggttctggatgt

SEQ ID NO: 618 - DNA, 39 bp, synthetic construct
  source 1..39: mol_type = other DNA
cgactcaagtgtgtggagtcttttctgggttctggatgt

SEQ ID NO: 619 - DNA, 39 bp, synthetic construct
  source 1..39: mol_type = other DNA
cgactcaagtgtgtggagtgcgttctgggttctggatgt

SEQ ID NO: 620 - DNA, 39 bp, synthetic construct
  source 1..39: mol_type = other DNA
cgactcaagtgtgtggagtggcttctgggttctggatgt

SEQ ID NO: 621 - DNA, 39 bp, synthetic construct
  source 1..39: mol_type = other DNA
cgactcaagtgtgtggagttctttctgggttctggatgt

SEQ ID NO: 622 - DNA, 39 bp, synthetic construct
  source 1..39: mol_type = other DNA
cgactcaagtgtgtggataacgttctgggttctggatgt

SEQ ID NO: 623 - DNA, 39 bp, synthetic construct
  source 1..39: mol_type = other DNA
cgactcaagtgtgtggataagcttctgggttctggatgt

SEQ ID NO: 624 - DNA, 39 bp, synthetic construct
  source 1..39: mol_type = other DNA
cgactcaagtgtgtggatacagttctgggttctggatgt

SEQ ID NO: 625 - DNA, 39 bp, synthetic construct
  source 1..39: mol_type = other DNA
cgactcaagtgtgtggatacgattctgggttctggatgt

SEQ ID NO: 626 - DNA, 39 bp, synthetic construct
  source 1..39: mol_type = other DNA
cgactcaagtgtgtggatagacttctgggttctggatgt

SEQ ID NO: 627 - DNA, 39 bp, synthetic construct
  source 1..39: mol_type = other DNA
cgactcaagtgtgtggatagcattctgggttctggatgt

SEQ ID NO: 628 - DNA, 39 bp, synthetic construct
  source 1..39: mol_type = other DNA
cgactcaagtgtgtggatcaagttctgggttctggatgt

SEQ ID NO: 629 - DNA, 39 bp, synthetic construct
  source 1..39: mol_type = other DNA
cgactcaagtgtgtggatcagattctgggttctggatgt

SEQ ID NO: 630 - DNA, 39 bp, synthetic construct
  source 1..39: mol_type = other DNA
cgactcaagtgtgtggatccgcttctgggttctggatgt

SEQ ID NO: 631 - DNA, 39 bp, synthetic construct
  source 1..39: mol_type = other DNA
cgactcaagtgtgtggatcgaattctgggttctggatgt

SEQ ID NO: 632 - DNA, 39 bp, synthetic construct
  source 1..39: mol_type = other DNA
cgactcaagtgtgtggatcgccttctgggttctggatgt

SEQ ID NO: 633 - DNA, 39 bp, synthetic construct
  source 1..39: mol_type = other DNA
cgactcaagtgtgtggatcgttttctgggttctggatgt

SEQ ID NO: 634 - DNA, 39 bp, synthetic construct
  source 1..39: mol_type = other DNA
cgactcaagtgtgtggatctgtttctgggttctggatgt

SEQ ID NO: 635 - DNA, 39 bp, synthetic construct
  source 1..39: mol_type = other DNA
cgactcaagtgtgtggatcttgttctgggttctggatgt

SEQ ID NO: 636 - DNA, 39 bp, synthetic construct
  source 1..39: mol_type = other DNA
cgactcaagtgtgtggatgaacttctgggttctggatgt

SEQ ID NO: 637 - DNA, 39 bp, synthetic construct
  source 1..39: mol_type = other DNA
cgactcaagtgtgtggatgacattctgggttctggatgt

SEQ ID NO: 638 - DNA, 39 bp, synthetic construct
  source 1..39: mol_type = other DNA
cgactcaagtgtgtggatgcaattctgggttctggatgt

SEQ ID NO: 639 - DNA, 39 bp, synthetic construct
  source 1..39: mol_type = other DNA
cgactcaagtgtgtggatgcggttctgggttctggatgt

SEQ ID NO: 640 - DNA, 39 bp, synthetic construct
  source 1..39: mol_type = other DNA
cgactcaagtgtgtggatgcttttctgggttctggatgt

SEQ ID NO: 641 - DNA, 39 bp, synthetic construct
  source 1..39: mol_type = other DNA
cgactcaagtgtgtggatggcgttctgggttctggatgt

SEQ ID NO: 642 - DNA, 39 bp, synthetic construct
  source 1..39: mol_type = other DNA
cgactcaagtgtgtggatgtctttctgggttctggatgt

SEQ ID NO: 643 - DNA, 39 bp, synthetic construct
  source 1..39: mol_type = other DNA
cgactcaagtgtgtggatgttcttctgggttctggatgt

SEQ ID NO: 644 - DNA, 39 bp, synthetic construct
  source 1..39: mol_type = other DNA
cgactcaagtgtgtggattcgtttctgggttctggatgt

SEQ ID NO: 645 - DNA, 39 bp, synthetic construct
  source 1..39: mol_type = other DNA
cgactcaagtgtgtggattctgttctgggttctggatgt

SEQ ID NO: 646 - DNA, 39 bp, synthetic construct
  source 1..39: mol_type = other DNA
cgactcaagtgtgtggattgctttctgggttctggatgt

SEQ ID NO: 647 - DNA, 39 bp, synthetic construct
  source 1..39: mol_type = other DNA
cgactcaagtgtgtggattgtcttctgggttctggatgt

SEQ ID NO: 648 - DNA, 42 bp, synthetic construct
  source 1..42: mol_type = other DNA
cgactcagattggtacaacagtagtcacatttctcagatcct

SEQ ID NO: 649 - DNA, 42 bp, synthetic construct
  source 1..42: mol_type = other DNA
cgactcagattggtacaacatgagtcacatttctcagatcct

SEQ ID NO: 650 - DNA, 42 bp, synthetic construct
  source 1..42: mol_type = other DNA
cgactcagattggtacaacgatagtcacatttctcagatcct

SEQ ID NO: 651 - DNA, 42 bp, synthetic construct
  source 1..42: mol_type = other DNA
cgactcagattggtacaacgtaagtcacatttctcagatcct

SEQ ID NO: 652 - DNA, 42 bp, synthetic construct
  source 1..42: mol_type = other DNA
cgactcagattggtacaactagagtcacatttctcagatcct

SEQ ID NO: 653 - DNA, 42 bp, synthetic construct
  source 1..42: mol_type = other DNA
cgactcagattggtacaactgaagtcacatttctcagatcct

SEQ ID NO: 654 - DNA, 42 bp, synthetic construct
  source 1..42: mol_type = other DNA
cgactcagattggtacaagactagtcacatttctcagatcct

SEQ ID NO: 655 - DNA, 42 bp, synthetic construct
  source 1..42: mol_type = other DNA
cgactcagattggtacaagatcagtcacatttctcagatcct

SEQ ID NO: 656 - DNA, 42 bp, synthetic construct
  source 1..42: mol_type = other DNA
cgactcagattggtacaagcatagtcacatttctcagatcct

SEQ ID NO: 657 - DNA, 42 bp, synthetic construct
  source 1..42: mol_type = other DNA
cgactcagattggtacaagctaagtcacatttctcagatcct

SEQ ID NO: 658 - DNA, 42 bp, synthetic construct
  source 1..42: mol_type = other DNA
cgactcagattggtacaagtacagtcacatttctcagatcct

SEQ ID NO: 659 - DNA, 42 bp, synthetic construct
  source 1..42: mol_type = other DNA
cgactcagattggtacaagtcaagtcacatttctcagatcct

SEQ ID NO: 660 - DNA, 42 bp, synthetic construct
  source 1..42: mol_type = other DNA
cgactcagattggtacaatacgagtcacatttctcagatcct

SEQ ID NO: 661 - DNA, 42 bp, synthetic construct
  source 1..42: mol_type = other DNA
cgactcagattggtacaatagcagtcacatttctcagatcct

SEQ ID NO: 662 - DNA, 42 bp, synthetic construct
  source 1..42: mol_type = other DNA
cgactcagattggtacaatcagagtcacatttctcagatcct

SEQ ID NO: 663 - DNA, 42 bp, synthetic construct
  source 1..42: mol_type = other DNA
cgactcagattggtacaatcgaagtcacatttctcagatcct

SEQ ID NO: 664 - DNA, 42 bp, synthetic construct
  source 1..42: mol_type = other DNA
cgactcagattggtacaatgacagtcacatttctcagatcct

SEQ ID NO: 665 - DNA, 42 bp, synthetic construct
  source 1..42: mol_type = other DNA
cgactcagattggtacaatgcaagtcacatttctcagatcct

SEQ ID NO: 666 - DNA, 42 bp, synthetic construct
  source 1..42: mol_type = other DNA
cgactcagattggtacacaagtagtcacatttctcagatcct

SEQ ID NO: 667 - DNA, 42 bp, synthetic construct
  source 1..42: mol_type = other DNA
cgactcagattggtacacaatgagtcacatttctcagatcct

SEQ ID NO: 668 - DNA, 42 bp, synthetic construct
  source 1..42: mol_type = other DNA
cgactcagattggtacacagatagtcacatttctcagatcct

SEQ ID NO: 669 - DNA, 42 bp, synthetic construct
  source 1..42: mol_type = other DNA
cgactcagattggtacacagtaagtcacatttctcagatcct

SEQ ID NO: 670 - DNA, 42 bp, synthetic construct
  source 1..42: mol_type = other DNA
cgactcagattggtacacatagagtcacatttctcagatcct

SEQ ID NO: 671 - DNA, 42 bp, synthetic construct
  source 1..42: mol_type = other DNA
cgactcagattggtacacatgaagtcacatttctcagatcct

SEQ ID NO: 672 - DNA, 42 bp, synthetic construct
  source 1..42: mol_type = other DNA
cgactcagattggtacaccgctagtcacatttctcagatcct

SEQ ID NO: 673 - DNA, 42 bp, synthetic construct
  source 1..42: mol_type = other DNA
cgactcagattggtacaccgtcagtcacatttctcagatcct

SEQ ID NO: 674 - DNA, 42 bp, synthetic construct
  source 1..42: mol_type = other DNA
cgactcagattggtacacctcgagtcacatttctcagatcct

SEQ ID NO: 675 - DNA, 42 bp, synthetic construct
  source 1..42: mol_type = other DNA
cgactcagattggtacacctgcagtcacatttctcagatcct

SEQ ID NO: 676 - DNA, 42 bp, synthetic construct
  source 1..42: mol_type = other DNA
cgactcagattggtacacgaatagtcacatttctcagatcct

SEQ ID NO: 677 - DNA, 42 bp, synthetic construct
  source 1..42: mol_type = other DNA
cgactcagattggtacacgataagtcacatttctcagatcct

SEQ ID NO: 678 - DNA, 42 bp, synthetic construct
  source 1..42: mol_type = other DNA
cgactcagattggtacacgcctagtcacatttctcagatcct

SEQ ID NO: 679 - DNA, 42 bp, synthetic construct
  source 1..42: mol_type = other DNA
cgactcagattggtacacgctcagtcacatttctcagatcct

SEQ ID NO: 680 - DNA, 42 bp, synthetic construct
  source 1..42: mol_type = other DNA
cgactcagattggtacacggtgagtcacatttctcagatcct

SEQ ID NO: 681 - DNA, 42 bp, synthetic construct
  source 1..42: mol_type = other DNA
cgactcagattggtacacgtaaagtcacatttctcagatcct

SEQ ID NO: 682 - DNA, 42 bp, synthetic construct
  source 1..42: mol_type = other DNA
cgactcagattggtacacgtccagtcacatttctcagatcct

SEQ ID NO: 683 - DNA, 42 bp, synthetic construct
  source 1..42: mol_type = other DNA
cgactcagattggtacacgtggagtcacatttctcagatcct

SEQ ID NO: 684 - DNA, 42 bp, synthetic construct
  source 1..42: mol_type = other DNA
cgactcagattggtacactaagagtcacatttctcagatcct

SEQ ID NO: 685 - DNA, 42 bp, synthetic construct
  source 1..42: mol_type = other DNA
cgactcagattggtacactagaagtcacatttctcagatcct

SEQ ID NO: 686 - DNA, 42 bp, synthetic construct
  source 1..42: mol_type = other DNA
cgactcagattggtacactccgagtcacatttctcagatcct

SEQ ID NO: 687 - DNA, 42 bp, synthetic construct
  source 1..42: mol_type = other DNA
cgactcagattggtacactcgcagtcacatttctcagatcct

SEQ ID NO: 688 - DNA, 42 bp, synthetic construct
  source 1..42: mol_type = other DNA
cgactcagattggtacactgaaagtcacatttctcagatcct

SEQ ID NO: 689 - DNA, 42 bp, synthetic construct
  source 1..42: mol_type = other DNA
cgactcagattggtacactgccagtcacatttctcagatcct

SEQ ID NO: 690 - DNA, 42 bp, synthetic construct
  source 1..42: mol_type = other DNA
cgactcagattggtacactgttagtcacatttctcagatcct

SEQ ID NO: 691 - DNA, 42 bp, synthetic construct
  source 1..42: mol_type = other DNA
cgactcagattggtacacttgtagtcacatttctcagatcct

SEQ ID NO: 692 - DNA, 42 bp, synthetic construct
  source 1..42: mol_type = other DNA
cgactcagattggtacagaactagtcacatttctcagatcct

SEQ ID NO: 693 - DNA, 42 bp, synthetic construct
  source 1..42: mol_type = other DNA
cgactcagattggtacagaatcagtcacatttctcagatcct

SEQ ID NO: 694 - DNA, 42 bp, synthetic construct
  source 1..42: mol_type = other DNA
cgactcagattggtacagacatagtcacatttctcagatcct

SEQ ID NO: 695 - DNA, 42 bp, synthetic construct
  source 1..42: mol_type = other DNA
cgactcagattggtacagactaagtcacatttctcagatcct

SEQ ID NO: 696 - DNA, 42 bp, synthetic construct
  source 1..42: mol_type = other DNA
cgactcagattggtacagatacagtcacatttctcagatcct

SEQ ID NO: 697 - DNA, 42 bp, synthetic construct
  source 1..42: mol_type = other DNA
cgactcagattggtacagatcaagtcacatttctcagatcct

SEQ ID NO: 698 - DNA, 42 bp, synthetic construct
  source 1..42: mol_type = other DNA
cgactcagattggtacagcaatagtcacatttctcagatcct

SEQ ID NO: 699 - DNA, 42 bp, synthetic construct
  source 1..42: mol_type = other DNA
cgactcagattggtacagcataagtcacatttctcagatcct

SEQ ID NO: 700 - DNA, 42 bp, synthetic construct
  source 1..42: mol_type = other DNA
cgactcagattggtacagcctcagtcacatttctcagatcct

SEQ ID NO: 701 - DNA, 42 bp, synthetic construct
  source 1..42: mol_type = other DNA
cgactcagattggtacagcggtagtcacatttctcagatcct

SEQ ID NO: 702 - DNA, 42 bp, synthetic construct
  source 1..42: mol_type = other DNA
cgactcagattggtacagcgtgagtcacatttctcagatcct

SEQ ID NO: 703 - DNA, 42 bp, synthetic construct
  source 1..42: mol_type = other DNA
cgactcagattggtacagctaaagtcacatttctcagatcct

SEQ ID NO: 704 - DNA, 42 bp, synthetic construct
  source 1..42: mol_type = other DNA
cgactcagattggtacagctccagtcacatttctcagatcct

SEQ ID NO: 705 - DNA, 42 bp, synthetic construct
  source 1..42: mol_type = other DNA
cgactcagattggtacagctggagtcacatttctcagatcct

SEQ ID NO: 706 - DNA, 42 bp, synthetic construct
  source 1..42: mol_type = other DNA
cgactcagattggtacaggcgtagtcacatttctcagatcct

SEQ ID NO: 707 - DNA, 42 bp, synthetic construct
  source 1..42: mol_type = other DNA
cgactcagattggtacaggctgagtcacatttctcagatcct

SEQ ID NO: 708 - DNA, 42 bp, synthetic construct
  source 1..42: mol_type = other DNA
cgactcagattggtacaggtcgagtcacatttctcagatcct

SEQ ID NO: 709 - DNA, 42 bp, synthetic construct
  source 1..42: mol_type = other DNA
cgactcagattggtacaggtgcagtcacatttctcagatcct

SEQ ID NO: 710 - DNA, 42 bp, synthetic construct
  source 1..42: mol_type = other DNA
cgactcagattggtacagtaacagtcacatttctcagatcct

SEQ ID NO: 711 - DNA, 42 bp, synthetic construct
  source 1..42: mol_type = other DNA
cgactcagattggtacagtacaagtcacatttctcagatcct

SEQ ID NO: 712 - DNA, 42 bp, synthetic construct
  source 1..42: mol_type = other DNA
cgactcagattggtacagtcaaagtcacatttctcagatcct

SEQ ID NO: 713 - DNA, 42 bp, synthetic construct
  source 1..42: mol_type = other DNA
cgactcagattggtacagtcggagtcacatttctcagatcct

SEQ ID NO: 714 - DNA, 42 bp, synthetic construct
  source 1..42: mol_type = other DNA
cgactcagattggtacagtcttagtcacatttctcagatcct

SEQ ID NO: 715 - DNA, 42 bp, synthetic construct
  source 1..42: mol_type = other DNA
cgactcagattggtacagtgcgagtcacatttctcagatcct

SEQ ID NO: 716 - DNA, 42 bp, synthetic construct
  source 1..42: mol_type = other DNA
cgactcagattggtacagtggcagtcacatttctcagatcct

SEQ ID NO: 717 - DNA, 42 bp, synthetic construct
  source 1..42: mol_type = other DNA
cgactcagattggtacagttctagtcacatttctcagatcct

SEQ ID NO: 718 - DNA, 42 bp, synthetic construct
  source 1..42: mol_type = other DNA
cgactcagattggtacataacgagtcacatttctcagatcct

SEQ ID NO: 719 - DNA, 42 bp, synthetic construct
  source 1..42: mol_type = other DNA
cgactcagattggtacataagcagtcacatttctcagatcct

SEQ ID NO: 720 - DNA, 42 bp, synthetic construct
  source 1..42: mol_type = other DNA
cgactcagattggtacatacagagtcacatttctcagatcct

SEQ ID NO: 721 - DNA, 42 bp, synthetic construct
  source 1..42: mol_type = other DNA
cgactcagattggtacatacgaagtcacatttctcagatcct

SEQ ID NO: 722 - DNA, 42 bp, synthetic construct
  source 1..42: mol_type = other DNA
cgactcagattggtacatagacagtcacatttctcagatcct

SEQ ID NO: 723 - DNA, 42 bp, synthetic construct
  source 1..42: mol_type = other DNA
cgactcagattggtacatagcaagtcacatttctcagatcct

SEQ ID NO: 724 - DNA, 42 bp, synthetic construct
  source 1..42: mol_type = other DNA
cgactcagattggtacatcaagagtcacatttctcagatcct

SEQ ID NO: 725 - DNA, 42 bp, synthetic construct
  source 1..42: mol_type = other DNA
cgactcagattggtacatcagaagtcacatttctcagatcct

SEQ ID NO: 726 - DNA, 42 bp, synthetic construct
  source 1..42: mol_type = other DNA
cgactcagattggtacatccgcagtcacatttctcagatcct

SEQ ID NO: 727 - DNA, 42 bp, synthetic construct
  source 1..42: mol_type = other DNA
cgactcagattggtacatcgaaagtcacatttctcagatcct

SEQ ID NO: 728 - DNA, 42 bp, synthetic construct
  source 1..42: mol_type = other DNA
cgactcagattggtacatcgccagtcacatttctcagatcct

SEQ ID NO: 729 - DNA, 42 bp, synthetic construct
  source 1..42: mol_type = other DNA
cgactcagattggtacatcgttagtcacatttctcagatcct

SEQ ID NO: 730 - DNA, 42 bp, synthetic construct
  source 1..42: mol_type = other DNA
cgactcagattggtacatctgtagtcacatttctcagatcct

SEQ ID NO: 731 - DNA, 42 bp, synthetic construct
  source 1..42: mol_type = other DNA
cgactcagattggtacatcttgagtcacatttctcagatcct

SEQ ID NO: 732 - DNA, 42 bp, synthetic construct
  source 1..42: mol_type = other DNA
cgactcagattggtacatgaacagtcacatttctcagatcct

SEQ ID NO: 733 - DNA, 42 bp, synthetic construct
  source 1..42: mol_type = other DNA
cgactcagattggtacatgacaagtcacatttctcagatcct

SEQ ID NO: 734 - DNA, 42 bp, synthetic construct
  source 1..42: mol_type = other DNA
cgactcagattggtacatgcaaagtcacatttctcagatcct

SEQ ID NO: 735 - DNA, 42 bp, synthetic construct
  source 1..42: mol_type = other DNA
cgactcagattggtacatgcggagtcacatttctcagatcct

SEQ ID NO: 736 - DNA, 42 bp, synthetic construct
  source 1..42: mol_type = other DNA
cgactcagattggtacatgcttagtcacatttctcagatcct

SEQ ID NO: 737 - DNA, 42 bp, synthetic construct
  source 1..42: mol_type = other DNA
cgactcagattggtacatggcgagtcacatttctcagatcct

SEQ ID NO: 738 - DNA, 42 bp, synthetic construct
  source 1..42: mol_type = other DNA
cgactcagattggtacatgtctagtcacatttctcagatcct

SEQ ID NO: 739 - DNA, 42 bp, synthetic construct
  source 1..42: mol_type = other DNA
cgactcagattggtacatgttcagtcacatttctcagatcct

SEQ ID NO: 740 - DNA, 42 bp, synthetic construct
  source 1..42: mol_type = other DNA
cgactcagattggtacattcgtagtcacatttctcagatcct

SEQ ID NO: 741 - DNA, 42 bp, synthetic construct
  source 1..42: mol_type = other DNA
cgactcagattggtacattctgagtcacatttctcagatcct

SEQ ID NO: 742 - DNA, 42 bp, synthetic construct
  source 1..42: mol_type = other DNA
cgactcagattggtacattgctagtcacatttctcagatcct

SEQ ID NO: 743 - DNA, 42 bp, synthetic construct
  source 1..42: mol_type = other DNA
cgactcagattggtacattgtcagtcacatttctcagatcct

SEQ ID NO: 744 - DNA, 41 bp, synthetic construct
  source 1..41: mol_type = other DNA
cgactcaagtgtgtggnnnnnngggtcagggttctggatat

SEQ ID NO: 745 - DNA, 41 bp, synthetic construct
  source 1..41: mol_type = other DNA
cgactcagattggtacnnnnnnacacsttkttcaggtcctc

SEQ ID NO: 746 - DNA, 39 bp, synthetic construct
  source 1..39: mol_type = other DNA
cgactcaagtgtgtggnnnnnnttctgggttctggatgt

SEQ ID NO: 747 - DNA, 42 bp, synthetic construct
  source 1..42: mol_type = other DNA
cgactcagattggtacnnnnnnagtcacatttctcagatcct

SEQ ID NO: 748 - DNA, 16 bp, synthetic construct
  source 1..16: mol_type = other DNA
cgactcaagtgtgtgg

SEQ ID NO: 749 - DNA, 16 bp, synthetic construct
  source 1..16: mol_type = other DNA
cgactcagattggtac

SEQ ID NO: 750 - DNA, 19 bp, synthetic construct
  source 1..19: mol_type = other DNA
gggtcagggttctggatat

SEQ ID NO: 751 - DNA, 19 bp, synthetic construct
  source 1..19: mol_type = other DNA
acacsttkttcaggtcctc

SEQ ID NO: 752 - DNA, 17 bp, synthetic construct
  source 1..17: mol_type = other DNA
ttctgggttctggatgt

SEQ ID NO: 753 - DNA, 20 bp, synthetic construct
  source 1..20: mol_type = other DNA
agtcacatttctcagatcct

SEQ ID NO: 754 - DNA, 33 bp, synthetic construct
  source 1..33: mol_type = other DNA
tcgtcggcagcgtcagatgtgtataagagacag

SEQ ID NO: 755 - DNA, 34 bp, synthetic construct
  source 1..34: mol_type = other DNA
gtctcgtgggctcggagatgtgtataagagacag